(19) United States
(12) Patent Application Publication
(10) Pub. No.: US 2026/0034105 A1
(43) Pub. Date: Feb. 5, 2026

(54) Generative AI-Assisted Identification of Novel PI3K-alpha Inhibitors

(71) Applicant: The First Affiliated Hospital of Xi'an Jiaotong University, Xi'an (CN)

(72) Inventors: Suxia HAN, Xi'an (CN); Yiping LI, Xi'an (CN); Yungang XU, Xi'an (CN); Yuesen LI, Xi'an (CN); Chengyi GAO, Xi'an (CN)

(73) Assignee: The First Affiliated Hospital of Xi'an Jiaotong University, Xi'an (CN)

(21) Appl. No.: 19/346,515

(22) Filed: Sep. 30, 2025

(51) Int. Cl. A61K 31/427; A61K 31/4184; C07D 235/22; C07D 417/12

(52) U.S. Cl. CPC A61K 31/427; A61K 31/4184; C07D 235/22; C07D 417/12

(57) ABSTRACT

DrugGPT is an innovative ligand design strategy based on the autoregressive model GPT (Generative Pre-trained Transformer), specifically optimized for exploring chemical space and discovering ligands for target proteins. The model was trained from scratch using a large dataset of protein-ligand binding pairs, enabling it to learn the structural and chemical rules of drug molecules and their interactions with proteins. We selected PI3Kα as an important anti-cancer drug target to demonstrate the ability of DrugGPT in exploring potential anti-cancer ligands. The present invention provides a novel class of PI3Kα inhibitors that exhibit exceptional therapeutic potential for the treatment, prevention, and management of a wide range of diseases and conditions associated with aberrant PI3Kα activity. These compounds are particularly promising for use in oncology, metabolic disorders, inflammatory diseases, and other conditions where PI3Kα signaling plays a critical role.

### BACKGROUND

DrugGPT is an innovative ligand design strategy based on the autoregressive model GPT (Generative Pre-trained Transformer), specifically optimized for exploring chemical space and discovering ligands for target proteins. The model was trained from scratch using a large dataset of protein-ligand binding pairs, enabling it to learn the structural and chemical rules of drug molecules and their interactions with proteins. We selected PI3Kα as an important anti-cancer drug target to demonstrate the ability of DrugGPT in exploring potential anti-cancer ligands. The present invention provides a novel class of PI3Kα inhibitors that exhibit exceptional therapeutic potential for the treatment, prevention, and management of a wide range of diseases and conditions associated with aberrant PI3Kα activity. These compounds are particularly promising for use in oncology, metabolic disorders, inflammatory diseases, and other conditions where PI3Kα signaling plays a critical role.

Phosphoinositide 3-kinases (PI3Ks) are a family of lipid kinases that play a critical role in cellular signaling pathways regulating cell growth, proliferation, survival, and metabolism.

Among the class I PI3Ks, the alpha isoform (PI3Kα) is of particular clinical significance due to its frequent dysregulation in human cancers and its role in mediating oncogenic signaling. PI3Kα is a heterodimeric enzyme composed of a catalytic subunit (p110α) and a regulatory subunit (p85α), which is activated by receptor tyrosine kinases (RTKs) and G-protein-coupled receptors (GPCRs). Upon activation, PI3Kα catalyzes the phosphorylation of phosphatidylinositol 4,5-bisphosphate (PIP2) to generate phosphatidylinositol 3,4,5-trisphosphate (PIP3), a secondary messenger that recruits and activates downstream effectors such as AKT and mTOR, driving oncogenic signaling.

PI3Kα is one of the most frequently mutated kinases in human cancers, with gain-of-function mutations in the PIK3CA gene (encoding p110α) observed in a wide range of malignancies, including breast, colorectal, endometrial, and gastric cancers. These mutations lead to constitutive activation of the PI3K pathway, promoting tumor growth, survival, and resistance to therapy. Additionally, PI3Kα is implicated in metabolic disorders and cardiovascular diseases due to its role in insulin signaling and angiogenesis. Given its central role in oncogenesis and other pathologies, PI3Kα has emerged as a promising therapeutic target for the development of targeted cancer therapies and other disease-modifying agents.

Several PI3K inhibitors have been developed and approved for clinical use, including pan-PI3K inhibitors (e.g., buparlisib), isoform-selective inhibitors (e.g., alpelisib for PI3Kα), and dual PI3K/mTOR inhibitors (e.g., dactolisib). Alpelisib, for instance, has shown efficacy in treating PIK3CA-mutated breast cancer and is approved in combination with fulvestrant for hormone receptor-positive, HER2-negative advanced breast cancer. However, current PI3K inhibitors face significant limitations, including:

Many inhibitors lack sufficient selectivity for PI3Kα, leading to inhibition of other PI3K isoforms (e.g., PI3Kβ, PI3Kδ, PI3Kγ) and resulting in adverse effects such as hyperglycemia, hepatotoxicity, and immune suppression.

Tumors often develop resistance to PI3K inhibitors through compensatory activation of parallel signaling pathways (e.g., MAPK/ERK) or feedback loops involving upstream RTKs.

While some inhibitors show promise in specific cancer types, their efficacy is often limited by tumor heterogeneity and the complexity of PI3K signaling networks.

Despite advances in PI3K-targeted therapies, there remains a critical need for novel inhibitors that address the shortcomings of existing compounds. Key unmet needs include: Improved Selectivity for PI3Kα, wherein development of inhibitors with enhanced isoform selectivity could minimize off-target effects and improve therapeutic windows; Drug Resistance: new compounds that target PI3Kα while simultaneously addressing resistance mechanisms (e.g., through dual inhibition of compensatory pathways) could provide more durable clinical responses; Expanded Therapeutic Applications: there is a need for PI3Kα inhibitors that are effective in a broader range of cancer types and stages, as well as in non-oncological conditions such as metabolic and cardiovascular diseases, and with improved pharmacokinetics, bioavailability, and/or tissue penetration to enhance the clinical utility of PI3Kα inhibitors.

### DrugGPT

To better address unmet clinical needs in the area of cancer biology and chemotherapy, there is a growing interest in AI-based lead-generation tools. DrugGPT is an innovative ligand design strategy based on the autoregressive model GPT (Generative Pre-trained Transformer), specifically optimized for exploring chemical space and discovering ligands for target proteins. The model leverages deep learning language models, which have demonstrated significant potential in protein design and biomedical text analysis, to generate novel molecules with potential binding affinity for specific proteins. DrugGPT significantly improves the efficiency of ligand design and offers a rapid and effective approach to drug discovery.

DrugGPT is built on the GPT-2 architecture, a state-of-the-art autoregressive language model. The model was trained from scratch using a large dataset of protein-ligand binding pairs, enabling it to learn the structural and chemical rules of drug molecules and their interactions with proteins.

We used the DrugGPT model to explore the potentialities of ligand design for PI3Kα inhibitors. In employing DrugGPT, we employed two inference methods: (1) observing the ligand design schemes that the model could autonomously generate without any input information, or (2) designing ligands based on a starting chemical structure. Disclosed are a novel family of PI3Kα inhibitors identified with the assistance of DrugGPT as a proof-of-concept exercise, demonstrating that chemical ligands identified with or without a starting chemical structure are capable of binding and inhibiting a target molecule.

To demonstrate the utility of the DrugGPT framework, novel chemical ligand structures identified as potential inhibitors of PI3Kα were generated by DrugGPT generative AI, and then 16 candidates were selected, synthesized and found to have PI3Kα inhibitory activity. An additional ligand, NU7427, which was originally identified as a DNA-PK inhibitor, was identified via the DrugGPT model as a potential PI3Kα inhibitor and shown to have PI3Kα inhibitory activity.

### DESCRIPTION OF FIGURES

FIG. 1: Chemical structures of compounds set forth in Table 1. This class of compounds consists of PI3Kα ligand with entirely novel scaffolds designed by AI.

FIG. 2: Chemical structures of compounds set forth in Table II. These compounds are PI3Kα ligands designed by AI through decorating based on Target 3-1 from Class 2.

FIG. 3: Synthesis of compound 8f725e.

FIG. 4: Synthesis of compound 750bc6.

FIG. 5: Synthesis of compound 47546a.

FIG. 6: Synthesis of compound b42c5d.

FIG. 7: Synthesis of compound c89310.

FIG. 8: Synthesis of compound dc081a.

FIG. 9: Synthesis of compound 7a0983-100.

FIG. 10: Synthesis of compound 89165b.

FIG. 11: Synthesis of compound b2607e.

FIG. 12: Synthesis of compound ecbde6.

FIG. 13: Synthesis of compound b0656f.

FIG. 14: Synthesis of compound f9de5d.

FIG. 15: Synthesis of compound 03adde.

FIG. 16: Synthesis of compound 55675a.

FIG. 17: Synthesis of compound b7a4b5.

FIG. 18: Synthesis of compound d0fd9a-0.

FIG. 19: Synthesis of target 3-1.

FIG. 20: Synthesis of target 3-2.

FIG. 21: Panels A-Z show IC₅₀ of AI-generated PI3Kα inhibitors.

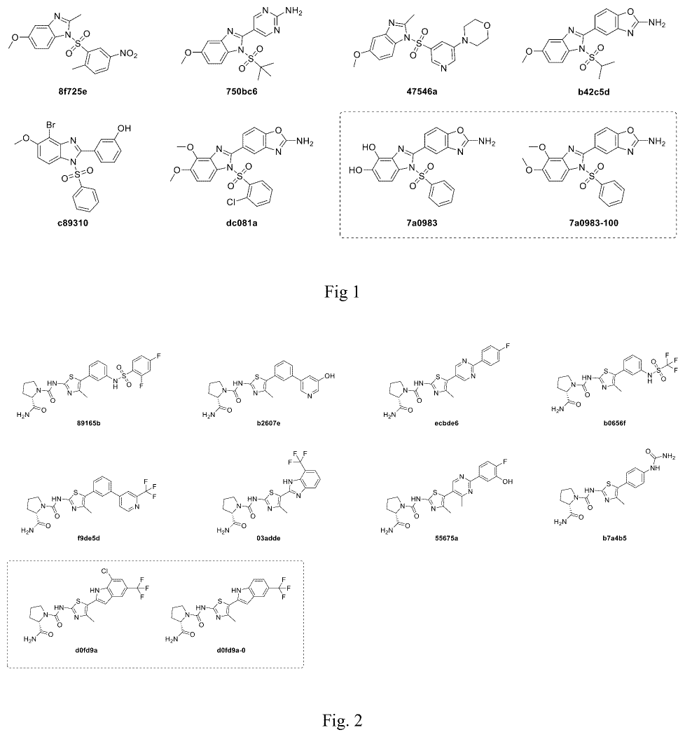

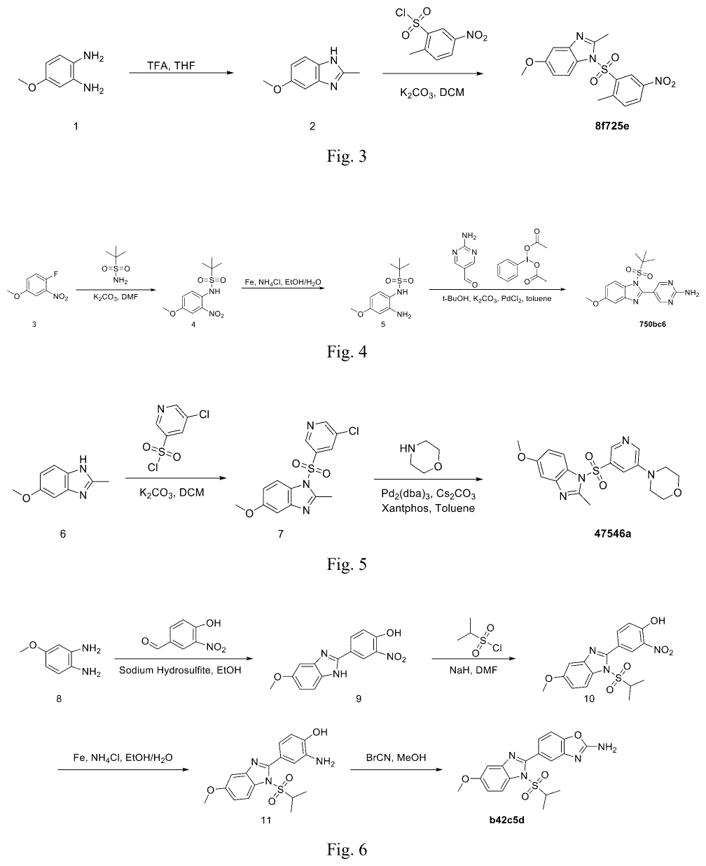

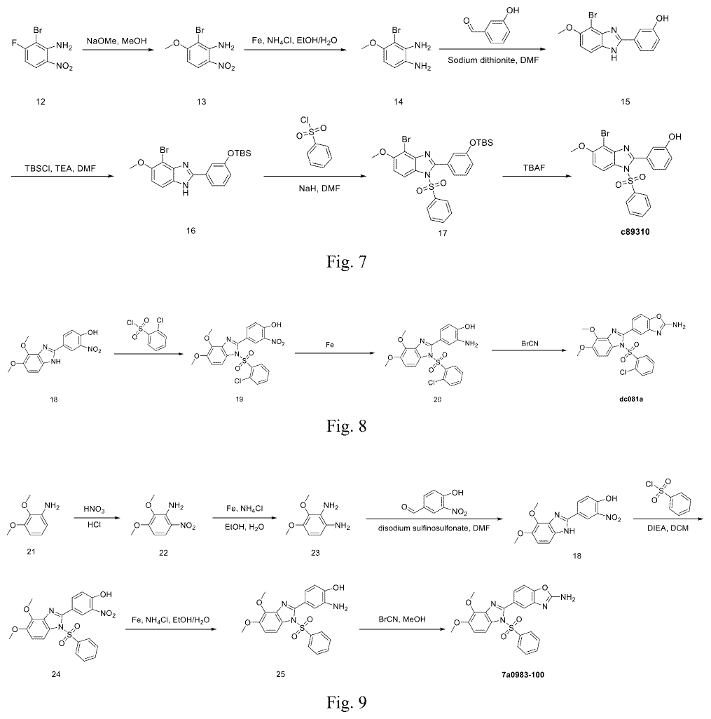

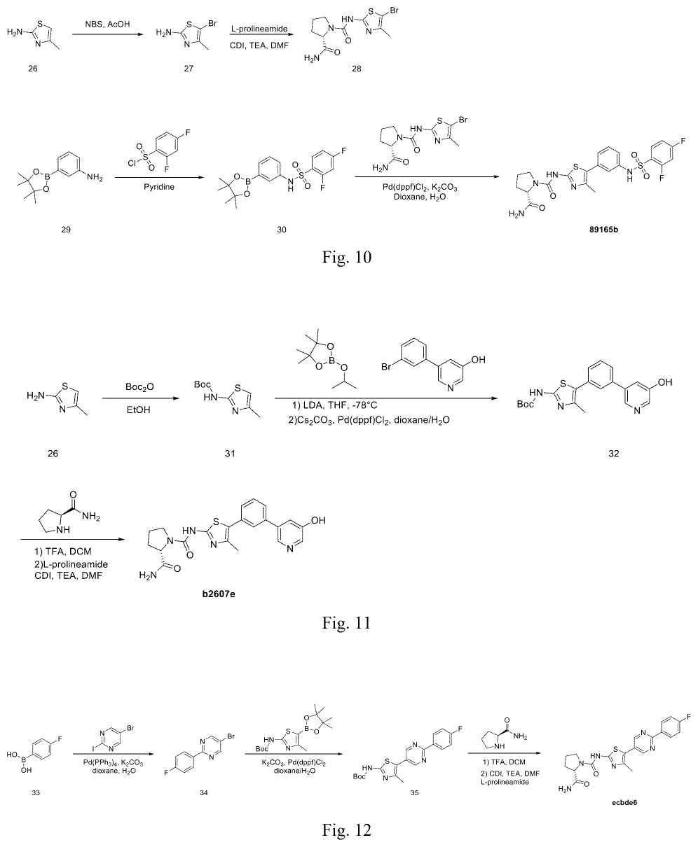

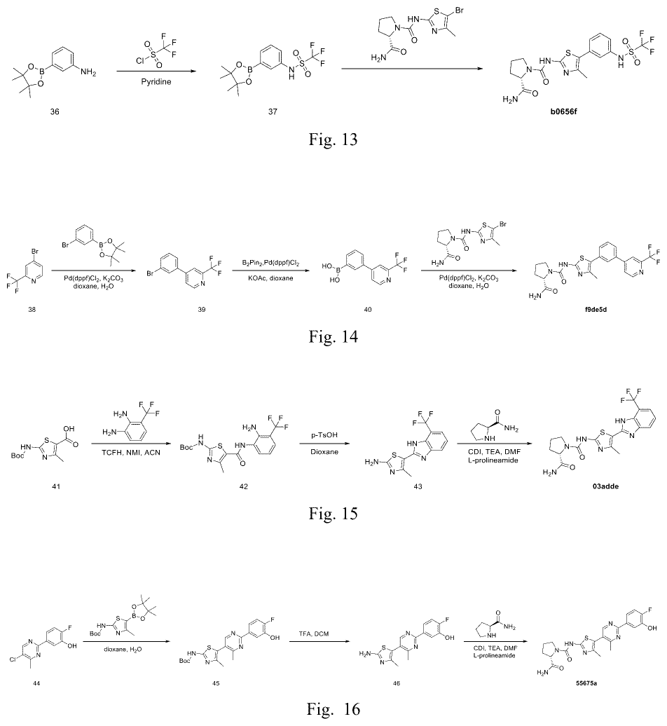

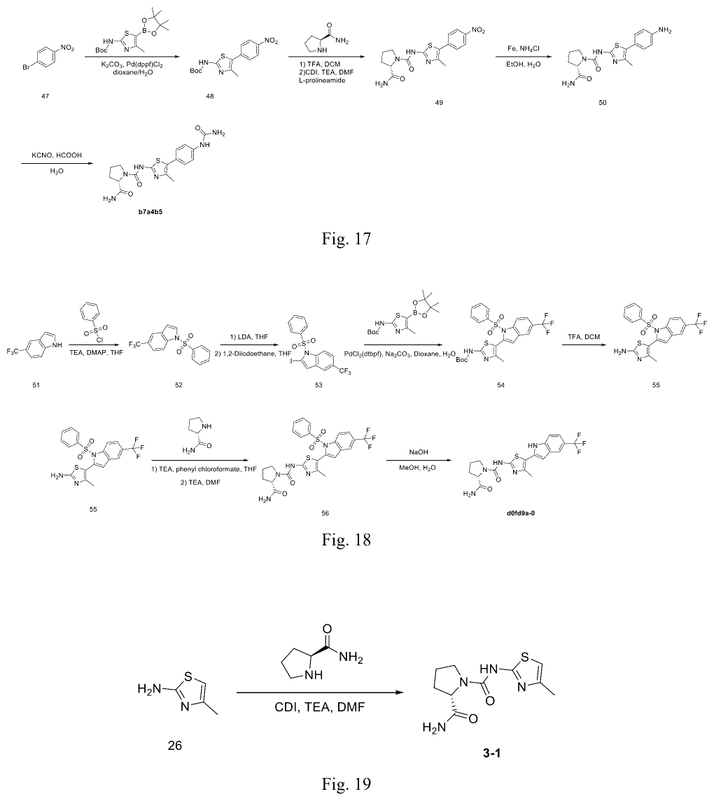

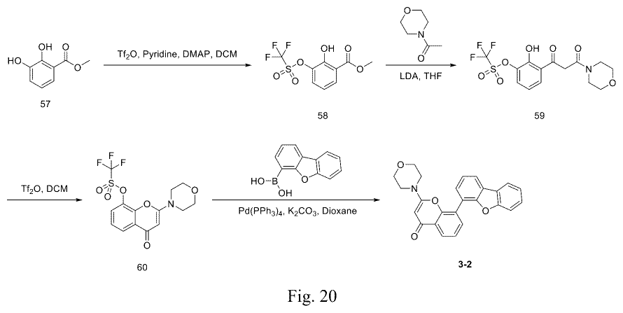

### DETAILED DESCRIPTION

PI3K inhibitors are typically small-molecule compounds that interact with the ATP-binding pocket of the p110α catalytic subunit. These compounds often feature heterocyclic scaffolds, such as morpholino-pyrimidines or thiazolidinediones, which confer binding affinity and selectivity. Key physicochemical properties, including molecular weight, lipophilicity, and hydrogen bonding capacity, influence the inhibitors' potency, selectivity, and drug-like characteristics. Advances in structural biology and computational modeling have enabled the rational design of inhibitors with optimized binding interactions and reduced off-target effects. DrugGPT leverages the significant corpus of structure/function data already developed in the area of PI3K inhibitors to identify novel structures and scaffolds likely to have PI3K inhibitory activity.

The development of novel PI3Kα inhibitors represents a significant opportunity to address unmet clinical needs in oncology and beyond. By leveraging insights into the structural and functional biology of PI3Kα, as well as lessons learned from existing inhibitors, next-generation compounds can be designed to achieve greater selectivity, efficacy, and safety. The present invention discloses a new class of PI3Kα inhibitors with the potential to overcome the limitations of current therapies, offering the potential for improved patient outcomes in a range of diseases driven by PI3K pathway dysregulation.

DrugGPT is built on the GPT-2 architecture, a state-of-the-art autoregressive language model. The model was trained from scratch using a large dataset of protein-ligand binding pairs, enabling it to learn the structural and chemical rules of drug molecules and their interactions with proteins. Key modifications include:

Tokenizer Optimization: The Byte Pair Encoding (BPE) algorithm was used to tokenize protein sequences and ligand SMILES representations. This approach reduces the complexity of representing chemical structures and protein sequences, enabling efficient exploration of chemical space.

Training Strategy: The training process was divided into two stages:

Stage 1: Training on ligand text data (SMILES representations) to help the model understand and generate chemical structures.

Stage 2: Training on protein-ligand pair text data to capture the binding relationships and generate ligands specific to target proteins.

Training Data: The model was trained on datasets from ZINC20 (a large database of drug-like compounds) and jglaser/binding_affinity (a database of protein-ligand interactions).

Tokenization of Chemical Space: DrugGPT uses a finite vocabulary of tokens to represent chemical structures, enabling efficient exploration of the vast chemical space. For example, only 5,373 tokens were needed to represent over 2 billion compounds in the ZINC20 database.

Autoregressive Generation: The model generates ligands in an autoregressive manner, predicting the next token in the sequence based on the previous tokens. This approach ensures high-quality and synthetically feasible molecules.

Ligand Prompts: Users can provide specific SMILES fragments (ligand prompts) to guide the generation of ligands with desired structural features, enhancing customization and optimization.

Efficiency: DrugGPT reduces the computational complexity of exploring chemical space by using tokenized representations of ligands and proteins.

Stability: Unlike Generative Adversarial Networks (GANs), which can suffer from mode collapse, DrugGPT's autoregressive training ensures a stable and reliable generation process.

Generalization: The model can adapt to different tasks, such as generating ligands for specific protein targets or optimizing existing compounds.

Ligand Design for Specific Proteins: DrugGPT was successfully used to generate ligands for proteins such as BCL-2 and ENPP2. For example, the model generated 73 potential ligands for BCL-2, a key anti-cancer target, and 167 ligands for ENPP2, a protein implicated in cancer and fibrosis. Novelty and Creativity: The model demonstrated the ability to generate both known ligands and entirely new compounds, showcasing its potential for innovative drug discovery.

Post-Processing: Generated ligands were post-processed using tools like Open Babel to convert SMILES representations into 3D structures for further analysis and validation.

### Identification of Lead Compounds

We used the DrugGPT model to explore the potentialities of ligand design for PI3Kα inhibitors. In employing DrugGPT, we employed two inference methods: (1) observing the ligand design schemes that the model could autonomously generate without any input information, or (2) designing ligands based on a starting chemical structure.

In the first inference mode, we did not provide any input information to the model but allowed it to autonomously generate ligand design schemes. Through this approach, we aimed to understand the kind of protein for which the model might design ligands in the absence of any prior information.

In the second inference mode, our goal was to design ligands that start with a specific SMILES representation. using the PI3Kα protein as an example, we used the SMILES representation starting with “CC1=C(SC(═N1)NC(═O)N2CCCC2C(═O)N)” as a prompt to generate specific ligands for PI3Kα.

Chemical ligands capable of binding and inhibiting PIK3CA were selected using the DrugGPT algorithm as described (DrugGPT: A GPT-based Strategy for Designing Potential Ligands Targeting Specific Proteins; Yuesen Li, Chengyi Gao, Xin Song, Xiangyu Wang, Yungang Xu, Suxia Han; bioRxiv 2023.06.29.543848; doi: https://doi.org/10.1101/2023.06.29.543848; incorporated by reference in its entirety).

Table 1 sets forth DrugGPT input strings. Table 2 sets a forth a representative selection of molecules identified based on a 0.95 p value in the DrugGPT model as PI3Kα binders in inference trials lacking structural input information for potential binding candidates. Table 3 sets a forth a representative selection of molecules identified based on a 0.95 p value in the DrugGPT model as PI3Kα binders in inference trials with the starting “CC1=C(SC(═N1)NC(═O)N2CCCC2C(═O)N)” SMILES string.

TABLE 1
Detailed Description of DrugGPT Command Line Usage and Parameters.

Exploring Novel Scaffold Ligands for PI3Kα Using De Novo Generation:
python drug_generator.py -f PI3Kα.fasta -n 100000 --top_k 40 --top_p 0.95 --no_limit -o
PI3K_100000-top_k_40-top_p_0.95 -b 32
Decorating Scaffold into PI3Kα Ligands via Ligand Prompt Generation:
python drug_generator.py -f PI3Kα.fasta -l CC1═C(SC(═N1)NC(═O)N2CCCC2C(═O)N) -
n 1000 --top_k 40 --top_p 0.95 -o PI3K_1000-top_k_40-top_p_0.95_l_CC1═C(SC(═N1)N
C(═O)N2CCCC2C(═O)N)
For detailed parameter information and command line usage, refer to the GitHub repository (github.com/LIYUESEN/druggpt).

### SMILES of Experimental Selected Molecules

TABLE 2
SMILES of Molecules Selected for Experimentation via De Novo Generation.

| Compound | |
|---|---|
| name | SMILES |
| 7a0983 | Oc1ccc2n(c(nc2c1O)-c1ccc2oc(N)nc2c1)S(═O)(═O)c1ccccc1 |
| c89310 | COc1ccc2n(c(nc2c1Br)-c1cccc(O)c1)S(═O)(═O)c1ccccc1 |
| 47546a | COc1ccc2n(c(C)nc2c1)S(═O)(═O)c1cncc(c1)N1CCOCC1 |
| b42c5d | COc1ccc2n(c(nc2c1)-c1ccc2oc(N)nc2c1)S(═O)(═O)C(C)C |
| dc081a | COc1ccc2n(c(nc2c1OC)-c1ccc2oc(N)nc2c1)S(═O)(═O)c1ccccc1Cl |
| 8f725e | COc1ccc2n(c(C)nc2c1)S(═O)(═O)c1cc(ccc1C)[N+]([O−])═O |
| 750bc6 | COc1ccc2n(c(nc2c1)-c1cnc(N)nc1)S(═O)(═O)C(C)(C)C |

TABLE 3
SMILES and Vina Score of Molecules Selected for Experimentation via Ligand Prompt Generation.

| Ligand Prompt: | CC1═C(SC(═N1)NC(═O)N2CCCC2C(═O)N) | |
|---|---|---|
| Compound name | SMILES | Vina score |
| d0fd9a | CC1═C(SC(═N1)NC(═O)N2CCCC2C(═O)N) | −12.2 |
| | 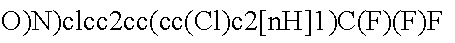 | |
| b2607e | CC1═C(SC(═N1)NC(═O)N2CCCC2C(═ | −11.6 |
| | O)N)clcccc(c1)-clcncc(O)c1 | |
| 55675a | CC1═C(SC(═N1)NC(═O)N2CCCC2C(═ | −11.6 |
| | O)N)clcnc(nc1C)-c1ccc(F)c(O)c1 | |
| 03adde | CC1═C(SC(═N1)NC(═O)N2CCCC2C(═ | −11.6 |
| | O)N)c1nc2cccc(c2[nH]1)C(F)(F)F | |
| f9de5d | CC1═C(SC(═N1)NC(═O)N2CCCC2C(═ | −11.6 |
| | O)N)clcccc(c1)-clccnc(c1)C(F)(F)F | |
| 89165b | CC1═C(SC(═N1)NC(═O)N2CCCC2C(═ | −11.6 |
| | O)N)clcccc(NS(═O)(═O)c2ccc(F)cc2F)c1 | |
| b7a4b5 | CC1═C(SC(═N1)NC(═O)N2CCCC2C(═ | −11.5 |
| | O)N)c1ccc(NC(N)═O)cc1 | |
| b0656f | CC1═C(SC(═N1)NC(═O)N2CCCC2C(═ | −11.4 |
| | O)N)clcccc(NS(═O)(═O)C(F)(F)F)c1 | |
| ecbde6 | CC1═C(SC(═N1)NC(═O)N2CCCC2C(═ | −11.3 |
| | O)N)clcnc(nc1)-c1ccc(F)cc1 | |

The bold parts of the SMILES represent the ligand prompt, and the parts with a gray background are generated by DrugGPT.

To test whether compounds identified by DrugGPT are indeed capable of binding and inhibiting a target molecule, selected PI3Kα inhibitors were synthesized and tested for PI3Kα inhibitory activity.

Among compounds selected without a ligand prompt, a group of sulfonyl-benzimidazole derivatives, sharing the core structure of Formula (I) (1-(sulfonyl)-1H-benzimidazole) was identified. A representative sample of these compounds, as set forth in Table 2, was selected for synthesis. The AI additionally identified as a potential PI3Kα inhibitor, Target 3-2, NU7427. NU7427 was originally identified and characterized pharmacologically as a DNA-PK inhibitor, but was demonstrated to have PI3Kα inhibitory activity in vitro (Table 4).

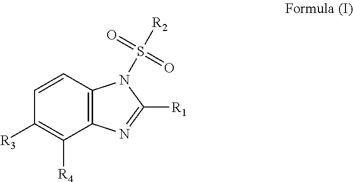

To prompt the generative AI to explore additional areas of chemical space, Target 3-1 was provided as a scaffold for a second iteration of ligand design via DrugGPT. The compounds set forth in Table 3 were selected for synthesis and characterization.

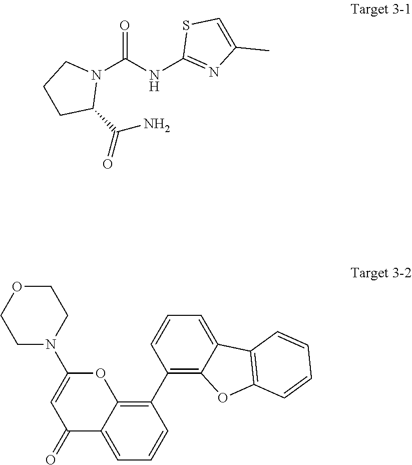 [SMILES: Cc1csc(NC(=O)N2CCC[C@H]2C(N)=O)n1 ; O=c1cc(N2CCOCC2)oc2c(-c3cccc4c3oc3ccccc34)cccc12]

### Methods

The chemical entities described herein can be synthesized according to one or more illustrative schemes herein and/or techniques well known in the art. Abbreviations and nomenclature used herein have their conventional meanings within the chemical and biological arts, as set forth in U.S. Pat. No. 8,193,182 B2, U.S. Pat. No. 9,221,795 B2, U.S. Pat. No. 9,255,103 B2, 9,629,843, and U.S. Pat. No. 10,112,932, each of which is incorporated herein by reference in their entirety.

Many of the optionally substituted starting compounds and other reactants are commercially available, e.g., from Aldrich Chemical Company (Milwaukee, Wis.) or can be readily prepared by those skilled in the art using commonly employed synthetic methodology.

The compounds of the invention can be synthesized by an appropriate combination of known synthetic methods in the art. The discussion below is offered to illustrate certain of the diverse methods available for use in making the compounds of the invention and is not intended to limit the scope of reactions or reaction sequences that can be used in preparing the compounds of the present invention.

### General Remarks and Instrumentation

Ethanol, methanol, toluene, ethyl acetate, dichloromethane, 1,4-dioxane, THF, acetonitrile and DMF were obtained from standard suppliers and used without further purification. All chemicals were purchased from Bidepharm Technology Co. Ltd, Energy Chemical, or Titan Scientific Co. Ltd and used without further purification. Anhydrous solvents were purchased from Energy Chemical. All reactions were monitored by thin layer chromatography (TLC) or display by iodine reagent. Column chromatography was carried out using Biotage Isolera flash purification system under proper pressure and HPLC. 1H NMR and 13C NMR were recorded on a Bruker AV-400 spectrometer at 400 MHz, respectively. Coupling constants (J) are expressed in hertz (Hz). Chemical shifts (δ) of NMR are reported in parts per million (ppm) units. Low resolution ESI-MS readings were recorded on an Agilent 1200-G6410A mass spectrometer. High-resolution mass spectra were recorded on Q-TOF Premier mass spectrometer (Micromass, Manchester, UK). The purity of final compounds was assessed by HPLC. HPLC analysis was performed on the Waters e2695 HPLC system with the use of a phenomenex-C18 reversed-column (4.6 mm×150 mm, 5 μm). The binary solvent system (A/B) was as follows: water (A) and MeCN (B). The absorbance was detected at 285 nm and the flow rate was 1.2 mL/min. The purity of compounds was determined to be over 95% by HPLC analysis

### Abbreviations

DCM: Dichloromethane; PE: petroleum ether; EA: Ethyl acetate; THF: Tetrahydrofuran; DMF: N,N-Dimethylformamide; EtOH: Ethanol; MeOH: Methanol; AcOH: Acetic acid; MeCN: Acetonitrile; TEA: Triethylamine; DME: 1,2-Dimethoxyethane; TCFH: Chloro-N,N,N′,N′-tetramethylformamidinium hexafluorophosphate; NMI: 1-Methylimidazole; K2CO3: Potassium carbonate; Na2CO3: Sodium carbonate; Cs2CO3: Cesium carbonate; NaH: Sodium hydride; NH4Cl: Ammonium chloride; BrCN: Cyanogen bromide; NaOMe: Sodium Methoxide; TBAF: Tetrabutylammonium fluoride; HNO3: Nitric acid; HCl: Hydrochloric acid; DIEA: N,N-Diisopropylethylamine; Fe: Iron; NBS: N-Bromosuccinimide; AcOK: Potassium Acetate; CDI: 1,1′-Carbonyldiimidazole; TFA: Trifluoroacetic acid; Pd(dppf)C12: [1,1′-Bis(diphenylphosphino)ferrocene]dichloropalladium(II); LDA: Lithium diisopropylamide; Pd(PPh3)4: Tetrakis(triphenylphosphine)palladium; B2Pin2: Bis(pinacolato)diboron; p-TsOH: p-toluenesulfonic acid; KCNO: Potassium cyanate; HCOOH: Formic acid; DMAP: 4-Dimethylaminopyridine; Pd(dtbpf)Cl2: 1,1′-Bis (di-t-butylphosphino)ferrocene palladium dichloride; NaOH: Sodium hydroxide; Tf2O: Trifluoroacetic anhydride; Na2SO4: Sodium sulfate; tBuOH: Tertiary butanol; PdCl2: Palladium chloride; Pd2(dba)3: 1,5-Diphenylpenta-1,4-dien-3-one;palladium; XantPhos: 4,5-Bis(diphenylphosphino)-9,9-dimethylxanthene; Boc20: Di-tert-butyl dicarbonate; NH4HCO3: Ammonium hydrogen carbonate; XPhosPdG3: Methanesulfonato(2-dicyclohexylphosphino-2′,4′,6′-tri-i-propyl-1,1′-biphenyl)(2′-amino-1,1′-biphenyl-2-yl)palladium(II); XPhos: 2-(dicyclohexylphosphino)-2′,4′,6′-tri-i-propyl-1,1′-biphenyl;

### Synthetic Routes

Procedure for the Synthesis of 8f725e

### 1. Synthesis of Compound-2

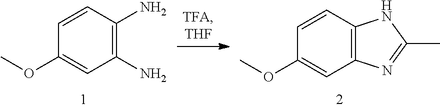

### 5-methoxy-2-methyl-4H-1,3-benzodiazole

To a stirred solution of 4-methoxybenzene-1,2-diamine (5.0 g, 36.2 mmol, 1.0 equiv.) and acetylacetone (7.2 g, 72.4 mmol, 2.0 equiv.) in THF (60 mL) was added TFA (2.0 g, 18.1 mmol, 0.5 equiv.) dropwise at 25° C. The resulting mixture was heated to 70° C. and stirred at 70° C. for additional 12 hours. The resulting mixture was cooled to 25° C. and concentrated under reduced pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 5-methoxy-2-methyl-1H-1,3-benzodiazole (700.0 mg, 11.93% yield) as a yellow solid.

LCMS (ESI): [M+H]⁺=163.00.

¹H NMR (400 MHz, Chloroform-d) δ 7.43 (d, J=8.7 Hz, 1H), 7.02 (d, J=2.4 Hz, 1H), 6.87-6.85 (m, 1H), 3.83 (s, 3H), 2.60 (s, 3H).

2. Synthesis of 8f725e

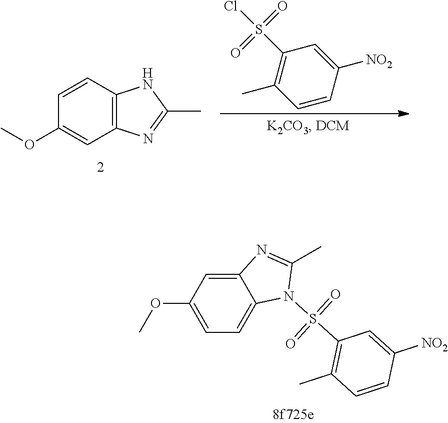

### 5-methoxy-2-methyl-1-(2-methyl-5-nitrobenzenesulfonyl)-1,3-benzodiazole

To a stirred solution of 5-methoxy-2-methyl-1H-1,3-benzodiazole (100.0 mg, 0.62 mmol, 1.0 equiv.) and K₂CO₃ (170.4 mg, 1.2 mmol, 2.0 equiv.) in DCM (1 mL) was added 2-methyl-5-nitrobenzenesulfonyl chloride (145.3 mg, 0.6 mmol, 1.0 equiv.) in portions at 0° C. The resulting mixture was stirred at 25° C. for 12 hours. The mixture was concentrated under reduced pressure. The residue was purified by prep-HPLC with the following conditions (Column: CHIRAL ART Amylose-SA, 2*25 cm, 5 m; Mobile Phase A: Hex(0.1% DEA)—HPLC, Mobile Phase B: MeOH:DCM=1:1—HPLC; Flow rate: 20 mL/min; Gradient: isocratic; Wave Length: 254 nm; RT1(min): 15.2; RT2(min): 18.4; Sample Solvent: MeOH; Injection Volume: 0.7 mL; Number Of Runs: 3) to afford 5-methoxy-2-methyl-1-(2-methyl-5-nitrobenzenesulfonyl)-1,3-benzodiazole (13.9 mg, 6.24% yield) as a white solid.

LCMS (ESI): [M+H]⁺=362.10.

¹H NMR (400 MHz, Chloroform-d) δ 8.81 (d, J=2.4 Hz, 1H), 8.39 (dd, J=8.4, 2.4 Hz, 1H), 7.66 (d, J=9.0 Hz, 1H), 7.54 (d, J=8.4 Hz, 1H), 7.20 (d, J=2.5 Hz, 1H), 6.95 (dd, J=9.0, 2.5 Hz, 1H), 3.87 (s, 3H), 2.71 (s, 3H), 2.55 (s, 3H).

¹³C NMR (100 MHz, Chloroform-d) δ 157.76, 151.74, 146.12, 145.50, 142.55, 138.82, 134.53, 128.48, 127.58, 124.51, 113.99, 113.73, 103.06, 55.75, 20.42, 17.01.

### Procedure for the Synthesis of 750bc6

### 1. Synthesis of Compound-4

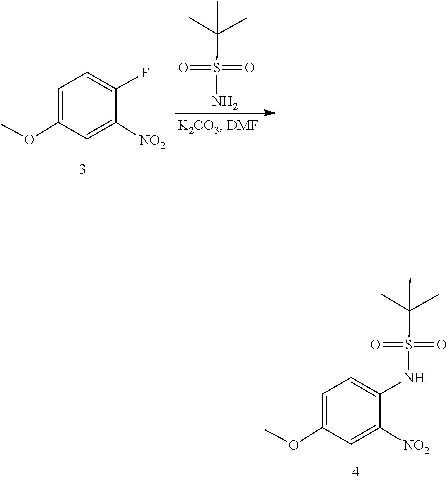

### N-(4-methoxy-2-nitrophenyl)-2-methylpropane-2-sulfonamide

To a stirred solution/mixture of 1-fluoro-4-methoxy-2-nitrobenzene (5.0 g, 29.2 mmol, 1.0 equiv.) and 2-methylpropane-2-sulfonamide (12.0 g, 87.6 mmol, 3.0 equiv.) in DMF (50 mL) was added K₂CO₃ (6.0 g, 43.8 mmol, 1.5 equiv.) in one portion at 25° C. under nitrogen atmosphere. The resulting mixture was heated to 140° C. and stirred at 140° C. for 10 hours under nitrogen atmosphere. The mixture was allowed to cool down to 25° C. The mixture was acidified to pH 5 with 1N HCl aqueous solution. The resulting mixture was extracted with EtOAc (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with PE/EA (3/1) to afford N-(4-methoxy-2-nitrophenyl)-2-methylpropane-2-sulfonamide (5.0 g, 59.3% yield) as a yellow solid.

LCMS (ESI): [M−H]⁻=287.05.

¹H NMR (400 MHz, DMSO-d₆) δ 9.48 (s, 1H), 7.60 (d, J=9.0 Hz, 1H), 7.46 (d, J=3.0 Hz, 1H), 7.28 (dd, J=9.0, 3.0 Hz, 1H), 3.83 (s, 3H), 1.29 (s, 9H).

### 2. Synthesis of Compound-5

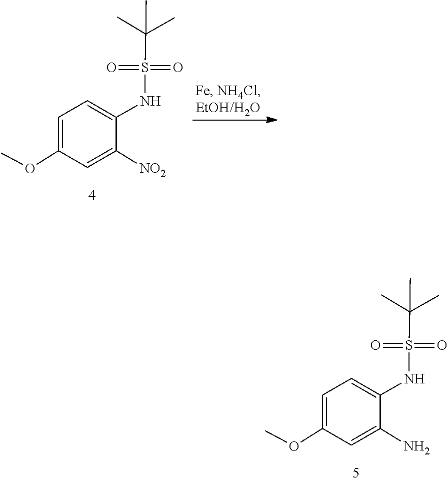 [SMILES: COc1ccc(NS(=O)(=O)C(C)(C)C)c([N+](=O)[O-])c1 ; COc1ccc(NS(=O)(=O)C(C)(C)C)c(N)c1]

### N-(2-amino-4-methoxyphenyl)-2-methylpropane-2-sulfonamide

To a stirred solution of N-(4-methoxy-2-nitrophenyl)-2-methylpropane-2-sulfonamide (2.0 g, 6.9 mmol, 1.0 equiv.) and NH4Cl (0.7 g, 13.8 mmol, 2.0 equiv.) in EtOH (20 mL) and H2O (4 mL) was added Fe (1.9 g, 34.6 mmol, 5.0 equiv.) in portions at 25° C. The resulting mixture was heated to 80° C. and stirred at 80° C. for 5 hours. The mixture was allowed to cool down to 25° C. The resulting mixture was filtered, the filter cake was washed with EtOH (3×20 mL). The filtrate was concentrated under reduced pressure. This resulted in N-(2-amino-4-methoxyphenyl)-2-methylpropane-2-sulfonamide (2.0 g, crude) as a red solid. The crude product was used for the next step.

LCMS (ESI): [M+H]⁺=259.10.

1H NMR (400 MHz, DMSO-d6) δ 8.51 (s, 1H), 7.07 (d, J=8.7 Hz, 1H), 6.29 (d, J=2.9 Hz, 1H), 6.12-6.09 (m, 1H), 5.13 (s, 2H), 3.64 (s, 3H), 1.27 (s, 9H).

### 3. Synthesis of 750bc6

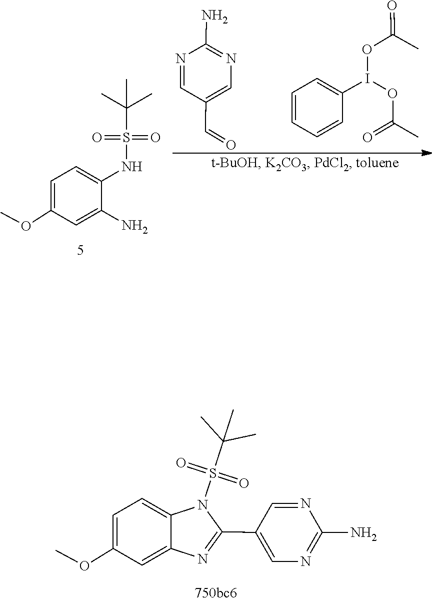

### 5-(1-(tert-butylsulfonyl)-5-methoxy-1H-benzo[d]imidazol-2-yl)pyrimidin-2-amine

To a stirred solution of N-(2-amino-4-methoxyphenyl)-2-methylpropane-2-sulfonamide (1.0 g, 3.8 mmol, 1.0 equiv.) in t-BuOH (10 mL) was added 2-aminopyrimidine-5-carbaldehyde (0.5 g, 4.6 mmol, 1.2 equiv.) in one portion at 25° C. under nitrogen atmosphere. The resulting mixture was heated to 90° C. and stirred at 90° C. for 2 hours under nitrogen atmosphere. The mixture was allowed to cool down to 25° C. The resulting mixture was concentrated under reduced pressure. To the above mixture was added (acetyloxy)(phenyl)-l\hat{ }[3]-iodanyl acetate (2.4 g, 7.7 mmol, 2.0 equiv.), K₂CO₃ (1.0 g, 7.7 mmol, 2.0 equiv.), PdCl₂ (68.6 mg, 0.3 mmol, 0.1 equiv.) and toluene (10 mL) in portions at 25° C. under nitrogen atmosphere. The resulting mixture was heated to 60° C. and stirred at 60° C. for additional 12 hours. The mixture was allowed to cool down to 25° C. The resulting mixture was concentrated under reduced pressure. The residue was purified by prep-HPLC with the following conditions (Column: YMC Triart C18 ExRs 5 μm, 30 mm*150 mm; Mobile Phase A: water (10 mmol/L NH4HCO3+0.1% NH4OH), Mobile Phase B: ACN; Flow rate: 60 mL/min; Gradient: 30% B to 45% B in 8 min; Wave Length: 254/220 nm; RT1(min): 7.8) to afford 5-[5-methoxy-1-(2-methylpropane-2-sulfonyl)-1,3-benzodiazol-2-yl]pyrimidin-2-amine (12.3 mg, 0.88% yield) as a white solid.

LCMS (ESI): [M+H]⁺=362.12.

¹H NMR (400 MHz, DMSO-d₆) δ 8.53 (s, 2H), 7.81 (d, J=9.1 Hz, 1H), 7.33 (d, J=2.5 Hz, 1H), 7.21 (s, 2H), 7.04 (dd, J=9.1, 2.6 Hz, 1H), 3.83 (s, 3H), 1.13 (s, 9H).

¹³C NMR (101 MHz, DMSO-d₆) δ 163.79, 160.05, 157.74, 151.90, 143.16, 129.22, 116.07, 114.45, 113.79, 103.03, 66.57, 56.08, 24.53.

### Procedure for the Synthesis of 47546a

### 1. Synthesis of Compound-7

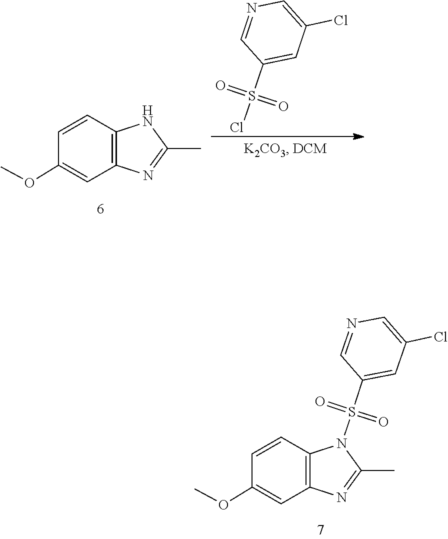

### 1-(5-chloropyridin-3-ylsulfonyl)-5-methoxy-2-methyl-1,3-benzodiazole

To a stirred solution of 5-methoxy-2-methyl-1H-1,3-benzodiazole (200.0 mg, 1.2 mmol, 1.0 equiv.) and K₂CO₃ (340.8 mg, 2.5 mmol, 2.0 equiv.) in DCM (2.0 mL) was added 5-chloropyridine-3-sulfonyl chloride (261.5 mg, 1.2 mmol, 1.0 equiv.) in portions at 0° C. The resulting mixture was allowed to 25° C. and stirred at 25° C. for 12 hours. The mixture was concentrated under reduced pressure. The residue was purified by silica gel column chromatography, eluted with PE/EA (1/1) to afford 1-(5-chloropyridin-3-ylsulfonyl)-5-methoxy-2-methyl-1,3-benzodiazole (225.0 mg, 89.3% yield) as a yellow oil.

LCMS (ESI): [M+H]⁺=338.05.

¹H NMR (400 MHz, DMSO-d₆) δ 9.27-9.23 (m, 1H), 9.01 (dd, J=4.7, 2.2 Hz, 1H), 8.76-8.69 (m, 1H), 7.87-7.51 (m, 1H), 7.41-7.17 (m, 1H), 6.97 (dt, J=9.0, 2.6 Hz, 1H), 3.82 (d, J=30.6 Hz, 3H), 2.80 (d, J=13.1 Hz, 3H).

### 2. Synthesis of 47546a

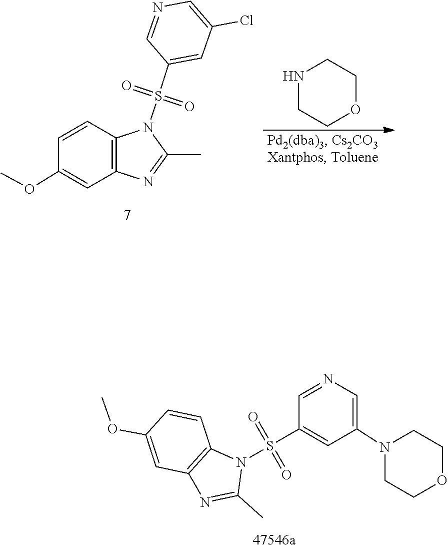 [SMILES: COc1ccc2c(c1)nc(C)n2S(=O)(=O)c1cncc(Cl)c1 ; C1COCCN1 ; COc1ccc2c(c1)nc(C)n2S(=O)(=O)c1cncc(N2CCOCC2)c1]

### 5-methoxy-2-methyl-1-[5-(morpholin-4-yl)pyridin-3-ylsulfonyl]-1,3-benzodiazole

To a stirred solution of 1-(5-chloropyridin-3-ylsulfonyl)-5-methoxy-2-methyl-1,3-benzodiazole (200.0 mg, 0.6 mmol, 1.0 equiv.) and Pd₂(dba)₃ (54.2 mg, 0.06 mmol, 0.1 equiv.) in 1,4-dioxane (2.0 mL) was added XantPhos (34.3 mg, 0.06 mmol, 0.1 equiv.) and Cs₂CO₃ (385.8 mg, 1.2 mmol, 2.0 equiv.) in portions at 25° C. under nitrogen atmosphere. The resulting mixture was heated to 100° C. and stirred at 100° C. for 12 hours. The mixture was concentrated under reduced pressure. The crude product was purified by prep-HPLC with the following conditions (Column: CHIRAL ART Amylose-SA, 2*25 cm, 5 m; Mobile Phase A: Hex(0.1% DEA)—HPLC, Mobile Phase B: MEOH:DCM=1:1; Flow rate: 20 mL/min; Gradient: isocratic; Wave Length: 254 nm; RT1(min): 7.2; RT2(min): 9.3; Sample Solvent: MEOH; Injection Volume: 1 mL; Number Of Runs: 3) to afford 5-methoxy-2-methyl-1-[5-(morpholin-4-yl)pyridin-3-ylsulfonyl]-1,3-benzodiazole (18.3 mg, 7.8% yield) as a white solid.

LCMS (ESI): [M+H]⁺=389.10.

¹H NMR (400 MHz, Methanol-d4) δ 8.54 (d, J=2.8 Hz, 1H), 8.49 (d, J=2.0 Hz, 1H), 7.71 (t, J=2.5 Hz, 1H), 7.54 (d, J=2.4 Hz, 1H), 7.48 (d, J=8.8 Hz, 1H), 7.01 (dd, J=8.8, 2.5 Hz, 1H), 3.90 (s, 3H), 3.82 (t, J=4.9 Hz, 4H), 3.28 (t, J=4.9 Hz, 4H), 2.81 (s, 3H).

¹³C NMR (100 MHz, Methanol-d4) δ 158.26, 150.73, 147.21, 142.13, 135.23, 134.90, 133.26, 119.40, 117.39, 113.13, 97.70, 65.93, 55.09, 15.47.

Procedure for the Synthesis of b42c5d

### 1. Synthesis of Compound-9

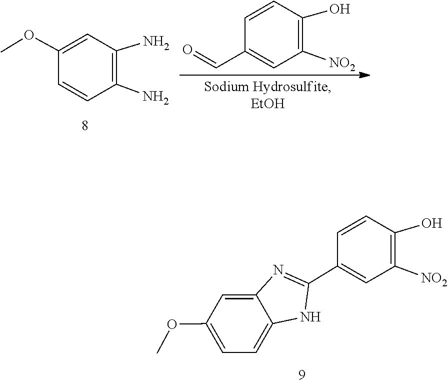

### 4-(5-methoxy-1H-benzo[d]imidazol-2-yl)-2-nitrophenol

A solution of 4-methoxybenzene-1,2-diamine (1.0 g, 7.2 mmol, 1.0 equiv.) in EtOH (20 mL) was treated with Sodium Hydrosulfite (516.6 mg, 2.9 mmol, 0.41 equiv.) in H₂O (1 mL) at 0° C. for 12 hours under nitrogen atmosphere followed by the addition of 4-hydroxy-3-nitrobenzaldehyde (1.21 g, 7.237 mmol, 1.0 equiv.) dropwise at 0° C. The resulting mixture was heated to 130° C. stirred at 130° C. for additional 4 hours. The resulting mixture was extracted with EtOAc (2×100 mL). The combined organic layers were washed with water (2×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water (10 mmol/L NH₄HCO₃), 0% to 100% gradient in 15 min; detector, UV 254 nm to afford 4-(5-methoxy-1H-benzo[d]imidazol-2-yl)-2-nitrophenol (1.5 g, 72.66% yield) as a yellow solid.

LCMS (ESI): [M+H]⁺=286.15

¹H NMR (400 MHz, DMSO-d₆) δ 12.78 (s, 1H), 8.66 (s, 1H), 8.26 (s, 1H), 7.46 (s, 1H), 7.18-7.06 (m, 2H), 6.84-6.81 (m, 1H), 3.81 (s, 3H).

2. Synthesis of compound-10

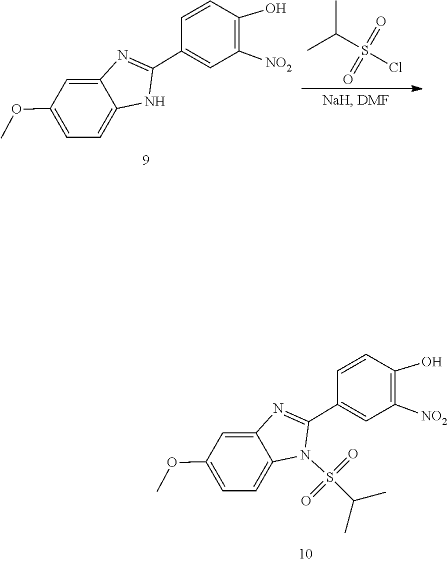

### 7-((5,6-difluoro-2-methyl-1H-benzo[d]imidazol-1-yl)methyl)-2-methyl-5H-thiazolo[3,2-a]pyrimidin-5-one

To a stirred solution of 4-(5-methoxy-1H-benzo[d]imidazol-2-yl)-2-nitrophenol (928 mg, 3.2 mmol, 1.0 equiv.) in DMF (10 mL) was added NaH (156.1 mg, 6.5 mmol, 2.0 equiv.) dropwise at 0° C. under nitrogen atmosphere. The resulting mixture was stirred for 30 minutes at 0° C. under nitrogen atmosphere. To the above mixture was added propane-2-sulfonyl chloride (556.6 mg, 3.9 mmol, 1.2 equiv.) dropwise at 0° C. The resulting mixture was stirred at 0° C. for additional 1 hour. The resulting mixture was quenched by water (10 mL) and extracted with EtOAc (2×50 mL). The combined organic layers were washed with water (2×50 mL), dried over anhydrous Na2SO4. After filtration, the filtrate was concentrated under reduced pressure. The residue was purified by reversed-phase flash chromatography with the following conditions: column, C18 silica gel; mobile phase, MeCN in Water (10 mmol/L NH4HCO3), 10% to 50% gradient in 10 min; detector, UV 254 nm to afford 4-(1-(isopropylsulfonyl)-5-methoxy-1H-benzo[d]imidazol-2-yl)-2-nitrophenol (700 mg, 54.98% yield) as a yellow solid.

LCMS (ESI): [M+H]⁺=392.10

1H NMR (400 MHz, DMSO-d6) δ 13.17 (s, 1H), 8.82 (d, J=2.3 Hz, 1H), 8.55-8.52 (m, 1H), 7.83 (d, J=8.6 Hz, 1H), 7.56-7.49 (m, 1H), 7.09 (d, J=7.9 Hz, 1H), 6.90 (d, J=9.1 Hz, 1H), 4.00-3.93 (m, 1H), 3.83 (s, 3H), 1.49 (d, J=6.7 Hz, 6H).

### 3. Synthesis of Compound-11

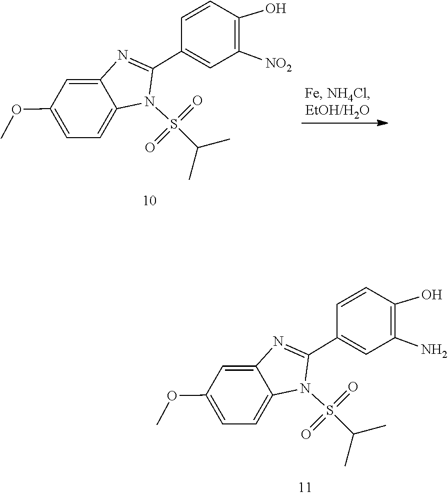 [SMILES: COc1ccc2c(c1)nc(-c1ccc(O)c([N+](=O)[O-])c1)n2S(=O)(=O)C(C)C ; COc1ccc2c(c1)nc(-c1ccc(O)c(N)c1)n2S(=O)(=O)C(C)C]

### 2-amino-4-(1-(isopropylsulfonyl)-5-methoxy-1H-benzo[d]imidazol-2-yl)phenol

To a stirred solution of 4-(1-(isopropylsulfonyl)-5-methoxy-1H-benzo[d]imidazol-2-yl)-2-nitrophenol (500 mg, 1.2 mmol, 1.0 equiv.) in EtOH (20 mL) and H₂O (4 mL) was added Fe (285.3 mg, 5.1 mmol, 4.0 equiv.) and ammonium chloride (170.8 mg, 3.2 mmol, 2.5 equiv.) at 25° C. The resulting mixture was heated to 80° C. and stirred at 80° C. for 1 hour. The mixture was cooled to 25° C. The resulting mixture was filtered, the filter cake was washed with EtOH (2×50 mL). The resulting mixture was concentrated under vacuum to afford 2-amino-4-(1-(isopropylsulfonyl)-5-methoxy-1H-benzo[d]imidazol-2-yl)phenol (400 mg, 86.64% yield) as a yellow solid.

LCMS (ESI): [M+H]⁺=362.10

¹H NMR (400 MHz, DMSO-d₆) δ 13.36 (s, 1H), 7.49-7.18 (m, 6H), 5.44 (s, 2H), 3.94 (s, 1H), 3.82 (s, 3H), 1.47 (s, 6H).

4. Synthesis of b42c5d

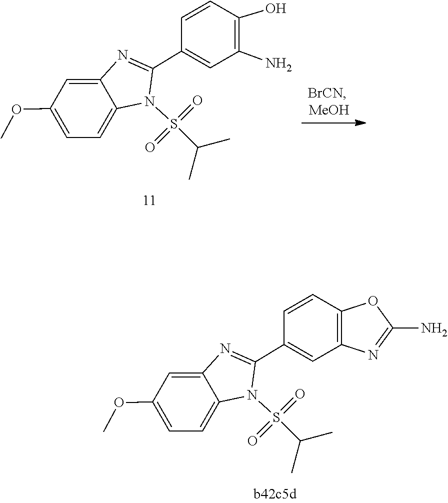 [SMILES: COc1ccc2c(c1)nc(-c1ccc(O)c(N)c1)n2S(=O)(=O)C(C)C ; COc1ccc2c(c1)nc(-c1ccc3oc(N)nc3c1)n2S(=O)(=O)C(C)C]

### 5-(1-(isopropylsulfonyl)-5-methoxy-1H-benzo[d]imidazol-2-yl)benzo[d]oxazol-2-amine

To a stirred solution of 2-amino-4-(1-(isopropylsulfonyl)-5-methoxy-1H-benzo[d]imidazol-2-yl)phenol (200 mg, 0.5 mmol, 1.0 equiv.) in methanol (2 mL) was added carbononitridic bromide (58.6 mg, 0.5 mmol, 1.0 equiv.) in H₂O (0.5 mL) dropwise at 5° C. The resulting mixture was stirred for 5 minutes at 5° C. The resulting mixture was allowed to 25° C. and stirred at 25° C. for 12 hours. The resulting mixture was directly purified by prep-HPLC with the following conditions (Column: Xbridge BEH Phenyl 5 μm, 30 mm*150 mm; Mobile Phase A: Water (10 mmol/L NH4HCO3+0.1% NH40H), Mobile Phase B: ACN; Flow rate: 60 mL/min; Gradient: 13% B to 31% B in 8 min; Wave Length: 254/220 nm; RT1(min): 7.15) to afford 5-(1-(isopropylsulfonyl)-5-methoxy-1H-benzo[d]imidazol-2-yl)benzo[d]oxazol-2-amine (12.8 mg, 5.99% yield) as a white solid.

LCMS (ESI): [M+H]⁺=387.15

¹H NMR (400 MHz, DMSO-d₆) δ 12.91 (s, 1H), 10.21 (s, 1H), 7.98 (s, 1H), 7.75 (d, J=8.7 Hz, 1H), 7.56-7.44 (m, 2H), 7.09 (s, 1H), 6.86 (d, J=8.9 Hz, 1H), 4.03 (p, J=6.8 Hz, 1H), 3.81 (s, 3H), 1.48 (d, J=6.8 Hz, 6H).

¹³C NMR (100 MHz, DMSO-d₆) δ 156.45, 150.01, 147.97, 138.42, 136.05, 130.67, 130.22, 128.86, 124.47, 124.06, 119.53, 115.67, 114.86, 112.44, 55.91, 53.54, 16.79.

Procedure for the Synthesis of c89310

### 1. Synthesis of Compound-13

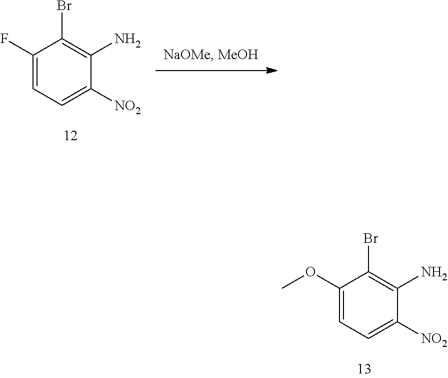

### 2-bromo-3-methoxy-6-nitroaniline

To a stirred solution of 2-bromo-3-fluoro-6-nitroaniline (1.0 g, 4.3 mmol, 1.0 equiv.) in methanol (20 mL) was added sodium methylate (0.5 g, 9.4 mmol, 2.2 equiv.) in portions at 25° C. under nitrogen atmosphere. The resulting mixture was heated to 25° C. and stirred for 1 hour at 90° C. under nitrogen atmosphere. The resulting mixture was cooled to 25° C. and concentrated under vacuum. The residue was purified by silica gel column chromatography, eluted with PE/EA (5/1) to afford 2-bromo-3-methoxy-6-nitroaniline (900 mg, 85.62% yield) as a yellow solid.

LCMS (ESI): [M+H]⁺=246.95

¹H NMR (400 MHz, DMSO-d₆) δ 8.16 (dd, J=9.6, 2.0 Hz, 1H), 7.23 (s, 2H), 6.63 (dd, J=9.7, 1.9 Hz, 1H), 3.96 (s, 3H).

2. Synthesis of compound-14

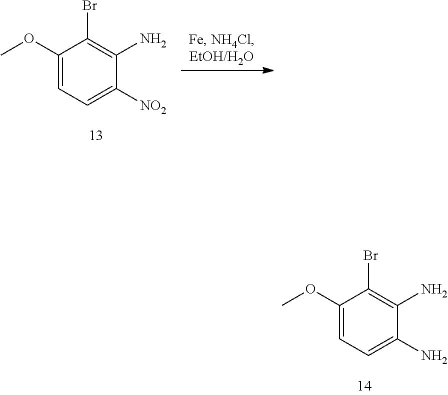

### 3-bromo-4-methoxybenzene-1,2-diamine

To a stirred solution of 2-bromo-3-methoxy-6-nitroaniline (1.0 g, 4.0 mmol, 1.0 equiv.) in EtOH (20 mL) was added Fe (1.1 g, 20.2 mmol, 5.0 equiv.) and ammonium chloride (1.0 g, 20.2 mmol, 5.0 equiv.) in one portion at 25° C. The resulting mixture was heated to 80° C. and stirred at 80° C. for 1 hour. The mixture was cooled to 25° C. The resulting mixture was filtered, the filter cake was washed with EtOH (2×10 mL). The filtrate was concentrated under reduced pressure. The residue was purified by reversed-phase flash chromatography with the following conditions: column, C18 silica gel; mobile phase, MeCN in Water, 0% to 50% gradient in 20 min; detector, UV 254 nm to afford 3-bromo-4-methoxybenzene-1,2-diamine (700 mg, 79.67% yield) as a yellow solid.

LCMS (ESI): [M+H]⁺=216.95

1H NMR (400 MHz, DMSO-d6) δ 6.51 (d, J=8.6 Hz, 1H), 6.17 (d, J=8.4 Hz, 1H), 4.65 (s, 4H), 3.67 (s, 3H).

### 3. Synthesis of Compound-15

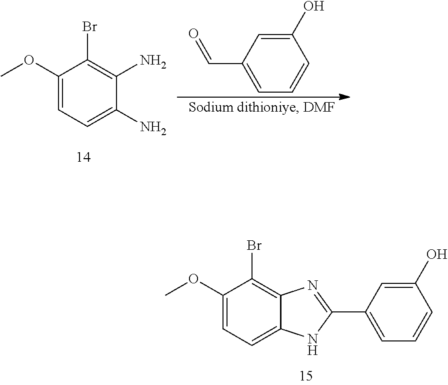 [SMILES: COc1ccc(N)c(N)c1Br ; O=Cc1cccc(O)c1 ; COc1ccc2[nH]c(-c3cccc(O)c3)nc2c1Br]

### 3-(4-bromo-5-methoxy-1H-benzo[d]imidazol-2-yl)phenol

To a stirred solution of 3-bromo-4-methoxybenzene-1,2-diamine (324 mg, 1.5 mmol, 1.0 equiv.) in EtOH (5 mL) was added Sodium Hydrosulfite (152 mg, 0.9 mmol, 0.6 equiv.) in H₂O (0.5 mL) at 25° C. under nitrogen atmosphere. The resulting mixture was stirred at 25° C. for 12 hours under nitrogen atmosphere. The resulting mixture was concentrated under vacuum. To the above mixture was added 3-hydroxybenzaldehyde (183 mg, 1.5 mmol, 1.0 equiv.) in DMF (5 mL) at 25° C. The resulting mixture was heated to 130° C. and stirred for additional 4 hours at 130° C. The resulting mixture was cooled to 0° C. and extracted with EtOAc (2×100 mL). The combined organic layers were washed with water (2×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 0% to 50% gradient in 10 min; detector, UV 254 nm to afford 3-(4-bromo-5-methoxy-1H-benzo[d]imidazol-2-yl)phenol (200 mg, 41.98% yield, 80% purity) as a yellow solid.

LCMS (ESI): [M+H]⁺=320.95.

¹H NMR (400 MHz, DMSO-d₆) δ 12.80 (d, J=148.3 Hz, 1H), 9.70 (d, J=32.0 Hz, 1H), 7.75-7.53 (m, 2H), 7.45 (d, J=8.6 Hz, 1H), 7.34 (dt, J=13.8, 7.9 Hz, 1H), 7.07 (t, J=8.6 Hz, 1H), 6.90 (p, J=4.4, 3.5 Hz, 1H), 3.89 (d, J=6.8 Hz, 3H).

### 4. Synthesis of Compound-16

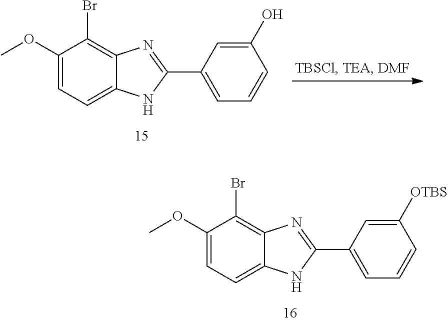 [SMILES: COc1ccc2[nH]c(-c3cccc(O)c3)nc2c1Br ; COc1ccc2[nH]c(-c3cccc(O[Si](C)(C)C(C)(C)C)c3)nc2c1Br]

### 4-bromo-2-(3-((tert-butyldimethylsilyl)oxy)phenyl)-5-methoxy-1H-benzo[d]imidazole

To a stirred solution of 3-(4-bromo-5-methoxy-1H-benzo[d]imidazol-2-yl)phenol (5.0 g, 15.7 mmol, 1.0 equiv.) in DCM (200 mL) was added tert-butyl(chloro)dimethylsilane (2.4 g, 15.7 mmol, 1.0 equiv.) and Et3N (4.8 g, 46.9 mmol, 3.0 equiv.) in portions at 25° C. The resulting mixture was stirred at 25° C. for 12 hours. The resulting mixture was filtered, the filter cake was washed with DCM (2×100 mL). The filtrate was concentrated under reduced pressure. The residue was purified by reversed-phase flash chromatography with the following conditions: column, C18 silica gel; mobile phase, MeCN in Water, 0% to 50% gradient in 20 min; detector, UV 254 nm to afford 4-bromo-2-(3-((tert-butyldimethylsilyl)oxy)phenyl)-5-methoxy-1H-benzo[d]imidazole (5.5 g, 81.00% yield) as a yellow solid.

LCMS (ESI): [M+H]⁺=432.90

1H NMR (400 MHz, DMSO-d6) δ 13.04-12.66 (d, J=150.4 Hz, 1H), 7.92-7.77 (m, 1H), 7.65-7.63 (m, 1H), 7.48-7.39 (m, 2H), 7.09 (d, J=8.5 Hz, 1H), 7.01-6.96 (m, 1H), 3.90-3.89 (m, 3H), 1.00 (s, 9H), 0.25 (s, 6H).

### 5. Synthesis of Compound-17

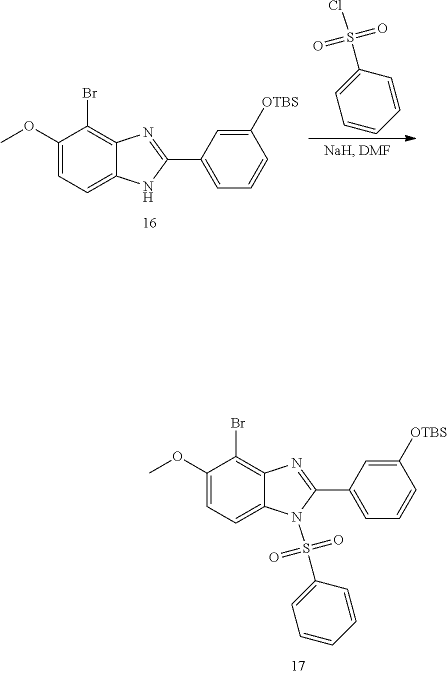 [SMILES: COc1ccc2[nH]c(-c3cccc(O[Si](C)(C)C(C)(C)C)c3)nc2c1Br ; O=S(=O)(Cl)c1ccccc1 ; COc1ccc2c(nc(-c3cccc(O[Si](C)(C)C(C)(C)C)c3)n2S(=O)(=O)c2ccccc2)c1Br]

### 4-bromo-2-(3-((tert-butyldimethylsilyl)oxy)phenyl)-5-methoxy-1-(phenylsulfonyl)-1H-benzo[d]imidazole

To a stirred solution of 4-bromo-2-(3-((tert-butyldimethylsilyl)oxy)phenyl)-5-methoxy-1H-benzo[d]imidazole (500 mg, 2.0 mmol, 1.0 equiv.) in EtOH (5 mL) was added NaH (213 mg, 8.9 mmol, 2.5 equiv.) in portions at 0° C. The resulting mixture was stirred at 0° C. for 30 minutes under nitrogen atmosphere. To the above mixture was added benzenesulfonyl chloride (1.3 g, 7.1 mmol, 2.0 equiv.) dropwise at 0° C. The resulting mixture was allowed to 25° C. and stirred at 25° C. for additional 2 hours. The reaction was quenched by the addition of water (100 mL) at 0° C. The resulting mixture was extracted with EtOAc (2×100 mL). The combined organic layers were washed with water (2×100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in Water, 0% to 100% gradient in 15 min; detector, UV 254 nm to afford 4-bromo-2-(3-((tert-butyldimethylsilyl)oxy)phenyl)-5-methoxy-1-(phenylsulfonyl)-1H-benzo[d]imidazole (1.2 g, 58.88% yield) as a yellow solid.

LCMS (ESI): [M+H]⁺=573.20

¹H NMR (400 MHz, DMSO-d₆) δ 8.02 (d, J=9.0 Hz, 1H), 7.76-7.69 (m, 1H), 7.58-7.51 (m, 4H), 7.41 (t, J=7.9 Hz, 1H), 7.31 (d, J=9.1 Hz, 1H), 7.19 (dt, J=7.9, 1.2 Hz, 1H), 7.14-7.07 (m, 2H), 3.93 (s, 3H), 0.98 (s, 9H), 0.23 (s, 6H).

6. Synthesis of c89310

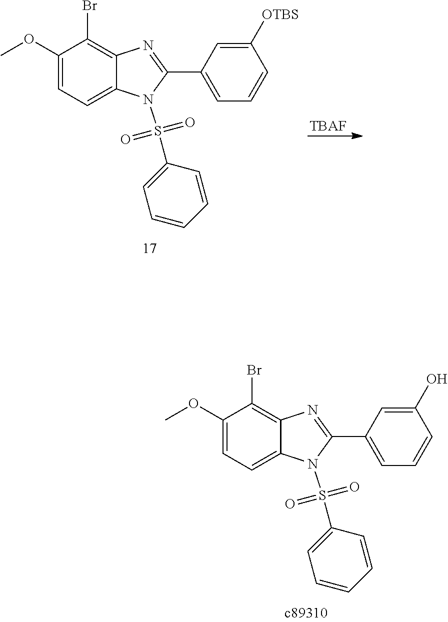

### 3-(4-bromo-5-methoxy-1-(phenylsulfonyl)-1H-benzo[d]imidazol-2-yl)phenol

Into a 8 mL vial were added 4-bromo-2-(3-((tert-butyldimethylsilyl)oxy)phenyl)-5-methoxy-1-(phenylsulfonyl)-1H-benzo[d]imidazole (200 mg, 0.3 mmol, 1.0 equiv.) and Tetrabutylammonium fluoride (3 mL in 1M THF, 0.1 mmol, 2.6 equiv.) at 25° C. The resulting mixture was stirred at 25° C. for 2 hours. The mixture was diluted with ethyl acetate (20 mL). The mixture was washed with water (5×10 mL). The organic phase was washed with brine. The organic phase was dried over Na₂SO₄, filtered and concentrated under reduce pressure. The residue was purified by reversed-phase flash chromatography with the following conditions: column, C18 silica gel; mobile phase, MeCN in Water (10 mmol/L NH₄HCO₃), 10% to 50% gradient in 10 min; detector, UV 254 nm. This resulted in 3-(4-bromo-5-methoxy-1-(phenylsulfonyl)-1H-benzo[d]imidazol-2-yl)phenol (15.9 mg, 9.93% yield) as a white solid.

LCMS (ESI): [M+H]⁺=459.00

¹H NMR (400 MHz, DMSO-d₆) δ 13.20-12.82 (m, 1H), 8.31-8.14 (d, J=46.9 Hz, 2H), 7.95-7.93 (m, 2H), 7.86-7.82 (m, 1H), 7.72-7.68 (m, 2H), 7.57-7.49 (m, 2H), 7.10 (t, J=9.4 Hz, 2H), 3.90 (s, 3H).

¹³C NMR (101 MHz, DMSO-d₆) δ 152.23, 151.34, 149.82, 143.74, 135.67, 134.65, 132.19, 131.23, 130.72, 130.39, 128.71, 125.97, 123.77, 120.68, 118.93, 111.20, 110.63, 57.65.

### Procedure for the Synthesis of Dc081a

### 1. Synthesis of Compound-19

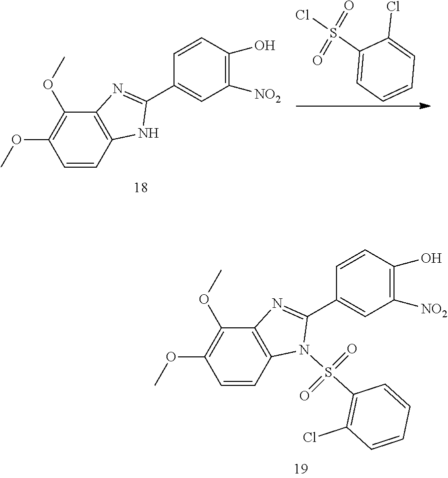 [SMILES: COc1ccc2[nH]c(-c3ccc(O)c([N+](=O)[O-])c3)nc2c1OC ; O=S(=O)(Cl)c1ccccc1Cl ; COc1ccc2c(nc(-c3ccc(O)c([N+](=O)[O-])c3)n2S(=O)(=O)c2ccccc2Cl)c1OC]

### 4-(1-((2-chlorophenyl)sulfonyl)-4,5-dimethoxy-1H-benzo[d]imidazol-2-yl)-2-nitrophenol

To a stirred mixture of 4-(4,5-dimethoxy-1H-1,3-benzodiazol-2-yl)-2-nitrophenol (5.0 g, 15.8 mmol, 1.0 equiv.) and DIEA (4.1 g, 31.7 mmol, 2.0 equiv.) in DCM (50 mL) was added 2-chlorobenzenesulfonyl chloride (3.3 g, 15.8 mmol, 1.0 equiv.) dropwise at 25° C. The resulting mixture was stirred at 25° C. for 2 hours. The resulting mixture was concentrated under reduced pressure. The residue was purified by silica gel column chromatography, eluted with PE/EA (1/1) to afford 4-[1-(2-chlorobenzenesulfonyl)-4,5-dimethoxy-1,3-benzodiazol-2-yl]-2-nitrophenol (3.4 g, 43.12% yield) as a yellow solid.

LCMS (ESI): [M+H]⁺=490.00.

¹H NMR (400 MHz, DMSO-d₆) δ 13.16 (d, J=10.0 Hz, 1H), 8.95-8.82 (m, 1H), 8.51-8.42 (m, 1H), 7.98-7.88 (m, 3H), 7.64-7.60 (m, 1H), 7.41-7.33 (m, 2H), 7.05 (d, J=8.8 Hz, 1H), 3.87-3.81 (m, 3H), 3.18-3.17 (m, 3H).

### 2. Synthesis of Compound-20

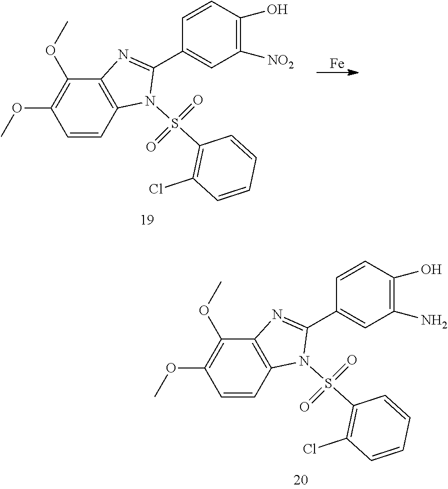

### 2-amino-4-(I-((2-chlorophenyl)sulfonyl)-4,5-dimethoxy-1H-benzo[d]imidazol-2-yl)phenol

To a stirred mixture of 4-[1-(2-chlorobenzenesulfonyl)-4,5-dimethoxy-1,3-benzodiazol-2-yl]-2-nitrophenol (3.4 g, 6.9 mmol, 1.0 equiv.) and NH₄Cl (0.7 g, 13.8 mmol, 2.0 equiv.) in EtOH (200 mL) and H₂O (40 mL) was added Fe (1.9 g, 34.7 mmol, 5.0 equiv.) in one portion at 25° C. The resulting mixture was heated to 80° C. and stirred at 80° C. for 12 hours. The resulting mixture was cooled down to 25° C. The resulting mixture was filtered, the filter cake was washed with EtOH (3×100 mL). The filtrate was concentrated under reduced pressure. This resulted in 2-amino-4-[1-(2-chlorobenzenesulfonyl)-4,5-dimethoxy-1,3-benzodiazol-2-yl]phenol (2.0 g, crude) as a yellow solid.

LCMS (ESI): [M+H]⁺=460.10.

¹H NMR (400 MHz, DMSO-d₆) δ 12.93 (s, 1H), 8.06-8.04 (m, 1H), 7.90-7.83 (m, 2H), 7.64-7.63 (m, 1H), 7.44 (s, 1H), 7.31 (s, 2H), 7.01 (d, J=8.5 Hz, 1H), 6.86 (d, J=8.5 Hz, 1H), 5.40 (s, 2H), 4.04 (s, 3H), 3.82 (s, 3H).

3. Synthesis of dc081a

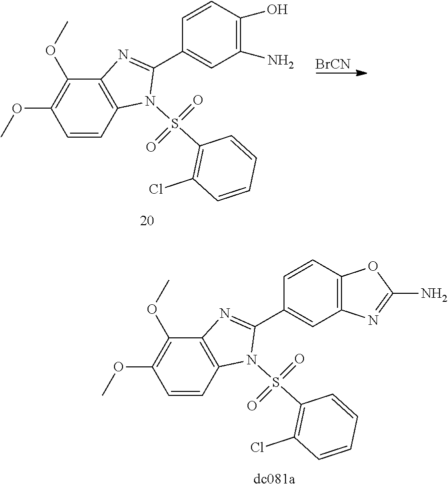

### 5-(1-((2-chlorophenyl)sulfonyl)-4,5-dimethoxy-1H-benzo[d]imidazol-2-yl)benzo[d]oxazol-2-amine

To a stirred solution of 2-amino-4-[1-(2-chlorobenzenesulfonyl)-4,5-dimethoxy-1,3-benzodiazol-2-yl]phenol (700.0 mg, 1.5 mmol, 1.0 equiv.) in MeOH (5 mL) was added BrCN (2.8 g, 26.4 mmol, 17.3 equiv.) in H₂O (10 mL) in one portion at 0° C. The resulting mixture was heated to 45° C. and stirred at 45° C. for 4 hours. The resulting mixture was concentrated under reduced pressure. The residue was purified by prep-HPLC with the following conditions (Column: Xbridge BEH Phenyl 5 μm, 30 mm*150 mm; Mobile Phase A: Water (10 mmol/L NH₄HCO₃+0.1% NH₄OH), Mobile Phase B: ACN; Flow rate: 60 mL/min; Gradient: 16% B to 37% B in 9 min; Wave Length: 254/220 nm; RT1(min): 7.03) to afford 5-[1-(2-chlorobenzenesulfonyl)-4,5-dimethoxy-1,3-benzodiazol-2-yl]-1,3-benzoxazol-2-amine (16.8 mg, 2.21% yield) as a light yellow solid.

LCMS (ESI): [M+H]⁺=485.10.

¹H NMR (400 MHz, Methanol-d₄) δ 8.04 (d, J=7.8, 1.0 Hz, 1H), 7.94 (d, J=2.0 Hz, 1H), 7.75-7.72 (m, 2H), 7.58-7.49 (m, 2H), 7.26 (d, J=8.8 Hz, 1H), 7.03 (dd, J=8.7, 3.9 Hz, 2H), 4.04 (s, 3H), 3.90 (s, 3H).

¹³C NMR (101 MHz, DMSO-d₆) δ 150.57, 147.80, 139.06, 136.89, 135.77, 133.45, 133.05, 132.36, 132.23, 129.73, 127.23, 122.76, 120.18, 116.18, 111.38, 109.78, 60.17, 56.80.

Procedure for the Synthesis of 7a0983-100

### 1. Synthesis of Compound-22

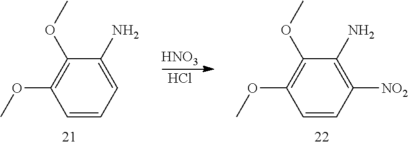

### 2,3-dimethoxy-6-nitroaniline

To a stirred solution of 2,3-dimethoxyaniline (1.0 g, 6.5 mmol, 1.0 equiv.) in acetic anhydride (10 mL) was added nitric acid (1 mL) dropwise at 0° C. The resulting mixture was stirred at 0° C. for 30 minutes. To the above mixture was added water (40 mL) and HCl (2 mL) in portions at 0° C. The resulting mixture was heated to 100° C. and stirred at 100° C. for additional 4 hours. The mixture was allowed to cool down to 25° C. The aqueous layer was extracted with EtOAc (3×20 mL). The combined organic layer was concentrated under reduced pressure. The residue was purified by reversed-phase flash chromatography with the following conditions: column, C18 silica gel; mobile phase, MeCN in water (10 mmol/L NH4HCO3), 10% to 50% gradient in 10 min; detector, UV 254 nm. This resulted in 2,3-dimethoxy-6-nitroaniline (500 mg, 38.65% yield) as a brown oil.

LCMS (ESI): [M+H]⁺=199.00.

1H NMR (400 MHz, DMSO-d6) δ 7.84 (d, J=9.8 Hz, 1H), 6.99 (s, 2H), 6.53 (d, J=9.8 Hz, 1H), 3.89 (s, 3H), 3.71 (s, 3H).

### 2. Synthesis of Compound-23

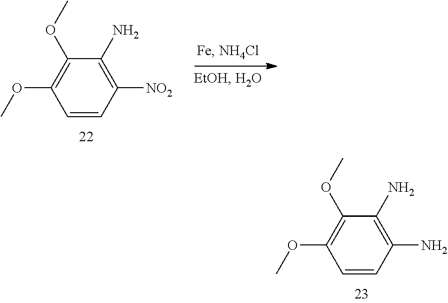

### 3,4-dimethoxybenzene-1,2-diamine

To a stirred solution of Fe (1.1 g, 20.2 mmol, 4.0 equiv.) and 2,3-dimethoxy-6-nitroaniline (1.0 g, 5.0 mmol, 1.0 equiv.) in EtOH (10 mL) was added NH4C1 (0.54 g, 10.1 mmol, 2.0 equiv.) in one portion at 25° C. The resulting mixture was heated to 25° C. and stirred at 60° C. for 12 hours. The resulting mixture was filtered, the filter cake was washed with ACN (2×20 mL). The combined filtrates were concentrated under reduced pressure. This resulted in 3,4-dimethoxybenzene-1,2-diamine (1.0 g, crude) as a black solid.

LCMS (ESI): [M+H]⁺=169.15.

1H NMR (400 MHz, Methanol-d4) δ 5.87 (s, 2H), 3.77 (s, 3H), 3.70 (s, 3H).

### 3. Synthesis of Compound-18

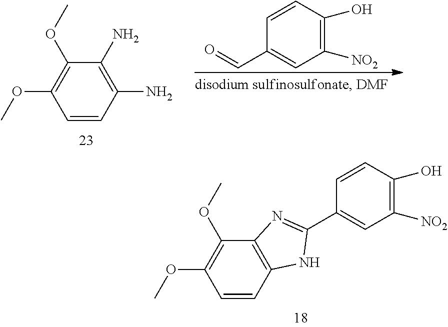 [SMILES: COc1ccc(N)c(N)c1OC ; O=Cc1ccc(O)c([N+](=O)[O-])c1 ; COc1ccc2[nH]c(-c3ccc(O)c([N+](=O)[O-])c3)nc2c1OC]

### 4-(4,5-dimethoxy-1H-benzo[d]imidazol-2-yl)-2-nitrophenol

A solution of 3,4-dimethoxybenzene-1,2-diamine (2.0 g, 11.9 mmol, 1.0 equiv.) in DMF (100 mL) was treated with disodium sulfinosulfonate (0.9 g, 4.8 mmol, 0.41 equiv.) in H₂O (10 mL) at 0° C. for 12 hours under nitrogen atmosphere followed by the addition of 4-formyl-2-nitrophenol (2.0 g, 11.9 mmol, 1.0 equiv.) in portions at 25° C. The resulting mixture was heated to 130° C. and stirred at 130° C. for 4 hours under nitrogen atmosphere. The resulting mixture was cooled to 25° C. and directly purified by reversed-phase flash chromatography with the following conditions: column, C18 silica gel; mobile phase, MeCN in Water (10 mmol/L NH₄HCO₃), 0% to 100% gradient in 10 min; detector, UV 254 nm to afford 4-(4,5-dimethoxy-1H-1,3-benzodiazol-2-yl)-2-nitrophenol (2.0 g, 53.35% yield) as a yellow solid.

LCMS (ESI): [M+H]⁺=316.15.

¹H NMR (400 MHz, Chloroform-d) δ 9.97 (s, 1H), 9.46 (s, 1H), 8.75 (d, J=2.2 Hz, 1H), 8.40 (dd, J=8.8, 2.2 Hz, 1H), 7.30 (d, J=8.8 Hz, 1H), 7.17 (d, J=8.8 Hz, 1H), 6.96 (d, J=8.6 Hz, 1H), 4.08 (s, 3H), 3.93 (s, 3H).

### 4. Synthesis of Compound-24

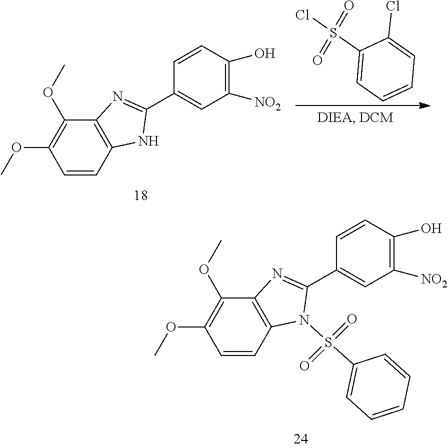 [SMILES: COc1ccc2[nH]c(-c3ccc(O)c([N+](=O)[O-])c3)nc2c1OC ; O=S(=O)(Cl)c1ccccc1Cl ; COc1ccc2c(nc(-c3ccc(O)c([N+](=O)[O-])c3)n2S(=O)(=O)c2ccccc2)c1OC]

### 4-(4,5-dimethoxy-1-(phenylsulfonyl)-1H-benzo[d]imidazol-2-yl)-2-nitrophenol

To a stirred solution of 4-(4,5-dimethoxy-1H-1,3-benzodiazol-2-yl)-2-nitrophenol (4.0 g, 12.6 mmol, 1.0 equiv.), benzenesulfonyl chloride (2.24 g, 12.6 mmol, 1.0 equiv.) and DIEA (3.28 g, 25.3 mmol, 2.0 equiv.) in DCM (40 mL). The resulting mixture was stirred at 25° C. for additional 1 hour. The resulting mixture was concentrated under vacuum. The residue was purified by flash chromatography with the following conditions: PE/EA=4/1, UV 254 nm.) to afford 4-[1-(benzenesulfonyl)-4,5-dimethoxy-1,3-benzodiazol-2-yl]-2-nitrophenol (1.2 g, 20.77% yield) as a yellow oil.

LCMS (ESI): [M+H]⁺=456.15.

¹H NMR (400 MHz, DMSO-d₆) δ 13.17 (d, J=16.4 Hz, 1H), 8.90-8.77 (m, 1H), 8.59-8.43 (m, 1H), 7.97-7.86 (m, 3H), 7.77-7.69 (m, 2H), 7.51-7.38 (m, 2H), 7.05 (d, J=8.8 Hz, 1H), 3.97-3.75 (m, 6H).

### 5. Synthesis of Compound-25

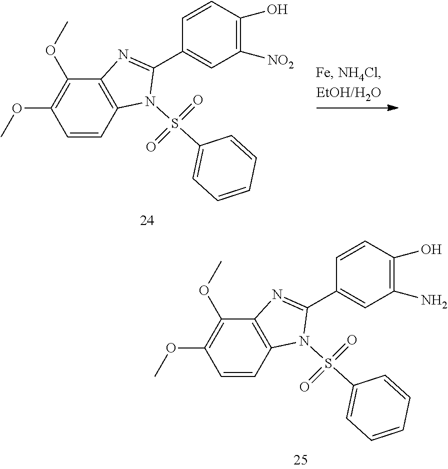 [SMILES: COc1ccc2c(nc(-c3ccc(O)c([N+](=O)[O-])c3)n2S(=O)(=O)c2ccccc2)c1OC ; COc1ccc2c(nc(-c3ccc(O)c(N)c3)n2S(=O)(=O)c2ccccc2)c1OC]

### 2-amino-4-(4,5-dimethoxy-1-(phenylsulfonyl)-1H-benzo[d]imidazol-2-yl)phenol

To a stirred solution of 4-[1-(benzenesulfonyl)-4,5-dimethoxy-1,3-benzodiazol-2-yl]-2-nitrophenol (1.3 g, 2.8 mmol, 1.0 equiv.) in EtOH (50 mL) and H₂O (10 mL) was added Fe (0.8 g, 14.2 mmol, 5.0 equiv.) and ammonium chloride (0.76 g, 14.270 mmol, 5.0 equiv.) in one portion at 25° C. The resulting mixture was stirred at 25° C. for 12 hours. The resulting mixture was filtered, the filter cake was washed with EtOH (2×50 mL). The filtrate was concentrated under reduced pressure. The resulting mixture was concentrated under vacuum. The crude product (580 mg) was used in the next step directly without further purification.

LCMS (ESI): [M+H]⁺=425.90

1H NMR (400 MHz, DMSO-d6) δ 12.83 (s, 1H), 7.99-7.97 (m, 2H), 7.83-7.79 (m, 1H), 7.67-7.63 (m, 2H), 7.51 (d, J=2.2 Hz, 1H), 7.26-7.15 (m, 2H), 7.10-6.99 (m, 2H), 5.33-5.29 (s, 2H), 4.06-4.03 (m, 3H), 3.82 (s, 3H).

### 6. Synthesis of 7a0893-100

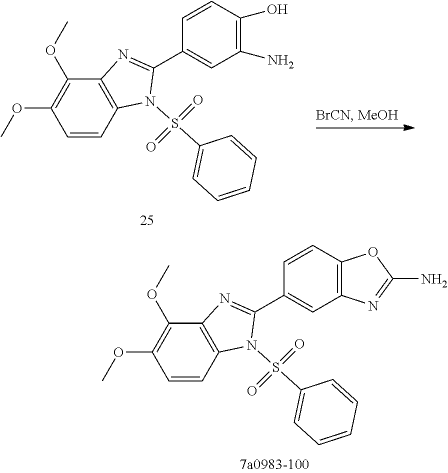 [SMILES: COc1ccc2c(nc(-c3ccc(O)c(N)c3)n2S(=O)(=O)c2ccccc2)c1OC ; COc1ccc2c(nc(-c3ccc4oc(N)nc4c3)n2S(=O)(=O)c2ccccc2)c1OC]

### 5-(4,5-dimethoxy-1-(phenylsulfonyl)-1H-benzo[d]imidazol-2-yl)benzo[d]oxazol-2-amine

To a stirred solution of 2-amino-4-[1-(benzenesulfonyl)-4,5-dimethoxy-1,3-benzodiazol-2-yl]phenol (400 mg, 0.9 mmol, 1.0 equiv.) in MeOH (4 mL) was added BrCN (1.6 g, 15.1 mmol, 16.0 equiv.) in portions at 0° C. The resulting mixture was heated to 38° C. stirred at 38° C. for 2 days. The resulting mixture was concentrated under reduced pressure. The crude product (200 mg) was purified by prep-HPLC with the following conditions (Column: YMC Triart C18 ExRs 5 μm, 30 mm*150 mm; Mobile Phase A: Water (10 mmol/L NH₄HCO₃+0.1% NH₃·H₂O), Mobile Phase B: ACN; Flow rate: 60 mL/min; Gradient: 18% B to 35% B in 9 min; Wave Length: 254/220 nm; RT1(min): 7.25) to afford 5-[1-(benzenesulfonyl)-4,5-dimethoxy-1,3-benzodiazol-2-yl]-1,3-benzoxazol-2-amine (12.8 mg, 3.12% yield) as a white solid.

LCMS (ESI): [M+H]⁺=451.10.

¹H NMR (400 MHz, Methanol-d₄) δ 7.90-7.88 (m, 2H), 7.80-7.73 (m, 2H), 7.63-7.56 (m, 3H), 7.30-7.25 (m, 2H), 7.03 (d, J=8.8 Hz, 1H), 4.04 (s, 3H), 3.90 (s, 3H).

¹³C NMR (101 MHz, DMSO-d₆) δ 150.65, 147.77, 139.15, 136.89, 136.05, 134.81, 134.72, 132.66, 129.63, 128.94, 128.57, 123.48, 120.00, 115.84, 111.35, 109.75, 60.20, 56.82.

### Procedure for the Synthesis of 89165b

### 1. Synthesis of Compound-27

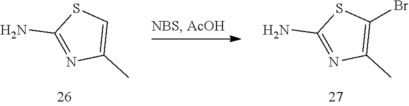

### 5-bromo-4-methylthiazol-2-amine

A solution of 2-thiazolamine, 4-methyl- (5.0 g, 43.8 mmol, 1.0 equiv.) in AcOH (100 mL) was treated with NBS (1.2 g, 65.7 mmol, 1.5 equiv.) at 25° C. The resulting mixture was stirred at 25° C. for 2 hours. The resulting mixture was concentrated under reduced pressure. The residue was purified by silica gel column chromatography, eluted with CH₂Cl₂/MeOH (12/1) to afford 5-bromo-4-methyl-1,3-thiazol-2-amine (3.0 g, 35.7% yield) as a yellow oil.

LCMS (ESI): [M+H]⁺=192.95.

¹H NMR (400 MHz, DMSO-d₆) δ 11.91 (s, 1H), 11.06 (s, 1H), 2.56 (s, 3H).

### 2. Synthesis of Compound-28

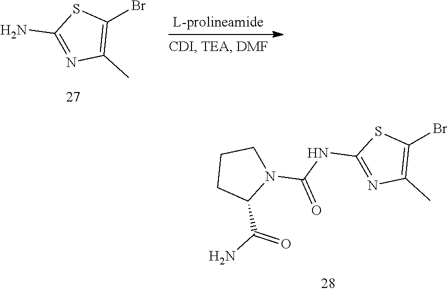

### (S)-N1-(5-bromo-4-methylthiazol-2-yl)pyrrolidine-1,2-dicarboxamide

A solution of 5-bromo-4-methyl-1,3-thiazol-2-amine (5.0 g, 25.9 mmol, 1.0 equiv.) in DMF (50 mL) was treated with Et₃N (7.9 g, 77.7 mmol, 3.0 equiv.) at 25° C. followed by the addition of 1-[(1H-imidazol-1-yl)carbonyl]-1H-imidazole (6.0 g, 38.8 mmol, 1.5 equiv.) in one portion at 25° C. The reaction mixture was heated to 70° C. and stirred for 2 hours at 70° C. The resulting mixture was slowly cooled to 25° C. To the above mixture was added prolinamide (4.0 g, 38.8 mmol, 1.5 equiv.) in portions at 25° C. The resulting mixture was stirred at 25° C. for additional 1 hour. The resulting mixture was extracted with CHCl₃ (3×50 mL). The combined organic layers were washed with brine (50 mL), dried over anhydrous Na₂SO₄. After filtration, the filtrate was concentrated under reduced pressure. The residue was purified by silica gel column chromatography, eluted with CH₂Cl₂/MeOH (10/1) to afford (S)-N1-(5-bromo-4-methylthiazol-2-yl)pyrrolidine-1,2-dicarboxamide (2.0 g, 23.21% yield) as an off-white solid.

LCMS (ESI): [M−H]-=331.00.

¹H NMR (400 MHz, DMSO-d₆) δ 10.92 (s, 1H), 7.38 (s, 1H), 6.96 (s, 1H), 4.26 (s, 1H), 3.60-3.52 (m, 1H), 3.42 (dt, J=9.8, 7.1 Hz, 1H), 2.19 (s, 3H), 2.07 (ddt, J=12.2, 8.1, 3.6 Hz, 1H), 1.86 (q, J=7.3, 6.9 Hz, 3H).

### 3. Synthesis of Compound-30

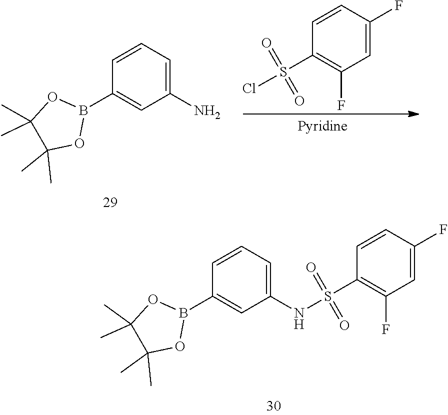 [SMILES: CC1(C)OB(c2cccc(N)c2)OC1(C)C ; O=S(=O)(Cl)c1ccc(F)cc1F ; CC1(C)OB(c2cccc(NS(=O)(=O)c3ccc(F)cc3F)c2)OC1(C)C]

### 2,4-difluoro-N-(3-(4,4,5,5-tetramethyl-1,3,2-dioxaborolan-2-yl)phenyl)benzenesulfonamide

A solution of 3-(4,4,5,5-tetramethyl-1,3,2-dioxaborolan-2-yl)aniline (2.0 g, 9.1 mmol, 1.0 equiv.) in Pyridine (1 mL) was treated with 2,4-difluorobenzenesulfonyl chloride (2.9 g, 13.7 mmol, 1.5 equiv.) at 25° C. The resulting mixture was stirred at 25° C. for 1 hour. The resulting mixture was concentrated under reduced pressure. The residue was purified by silica gel column chromatography, eluted with PE/EA (5/1) to afford 2,4-difluoro-N-(3-(4,4,5,5-tetramethyl-1,3,2-dioxaborolan-2-yl)phenyl)benzenesulfonamide (2.1 g, 58.41% yield) as an off-white solid.

LCMS (ESI): [M−H]-=394.05.

¹H NMR (400 MHz, DMSO-d₆) δ 10.61 (s, 1H), 7.86 (td, J=8.6, 6.2 Hz, 1H), 7.53 (ddd, J=10.6, 9.1, 2.5 Hz, 1H), 7.43-7.31 (m, 2H), 7.31-7.21 (m, 3H), 1.27 (s, 12H).

### 4. Synthesis of 89165b

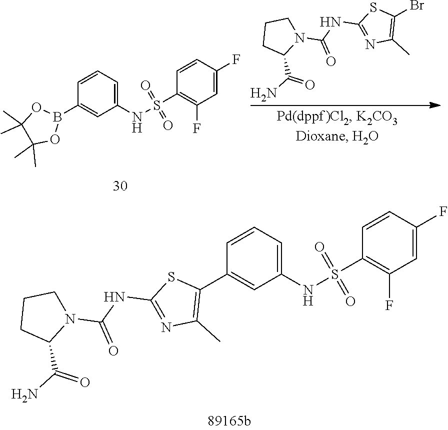

### (S)-N1-(5-(3-((2,4-difluorophenyl)sulfonamido)phenyl)-4-methylthiazol-2-yl)pyrrolidine-1,2-dicarboxamide

A solution of 2,4-difluoro-N-(3-(4,4,5,5-tetramethyl-1,3,2-dioxaborolan-2-yl)phenyl)benzenesul fonamide (200 mg, 0.5 mmol, 1.0 equiv.) in DME (2 mL) and H₂O (0.5 mL) was treated with Na₂CO₃ (161 mg, 1.5 mmol, 3.0 equiv.) and (2S)-N1-(5-bromo-4-methyl-1,3-thiazol-2-yl)pyrrolidine-1,2-dicarboxamide (252 mg, 0.8 mmol, 1.5 equiv.) at 25° C. followed by the addition of Pd(PPh₃)₄ (58 mg, 0.1 mmol, 0.1 equiv.) in portions at 25° C. The reaction mixture was heated to 80° C. and stirred at 80° C. for 16 hours under nitrogen atmosphere. The resulting mixture was slowly cooled to 25° C. The resulting mixture was filtered, the filter cake was washed with MeOH (2×5 mL). The combined filtrates were concentrated under reduced pressure. The crude product was purified by prep-HPLC with the following conditions (Column: XBridge BEH C185 μm, 19*250 mm; Mobile Phase A: water (10 mmol/L NH₄HCO₃+0.1% NH₃·H₂O), Mobile Phase B: ACN; Flow rate: 25 mL/min; Gradient: 16% B to 35% B in 10 min; Wave Length: 254 nm/220 nm; RT1(min): 9.45) to afford (S)-N1-(5-(3-((2,4-difluorophenyl)sulfonamido)phenyl)-4-methylthiazol-2-yl)pyrrolidine-1,2-dicarboxamide (12.5 mg, 4.81% yield) as a white solid.

LCMS (ESI): [M+H]⁺=522.10.

¹H NMR (400 MHz, DMSO-d₆) δ 10.71 (s, 2H), 8.11-7.81 (m, 1H), 7.58-7.49 (m, 1H), 7.45-7.15 (m, 4H), 7.12-6.85 (m, 3H), 4.29 (s, 1H), 3.64-3.55 (m, 1H), 3.55-3.38 (m, 1H), 2.22 (s, 3H), 2.17-1.98 (m, 1H), 1.98-1.70 (m, 3H).

¹³C NMR (101 MHz, DMSO) δ 174.46, 164.40, 160.64, 158.22, 138.44, 133.88, 132.97, 132.86, 130.24, 124.16, 119.58, 118.51, 112.97, 112.77, 106.83, 106.57, 106.32, 60.32, 46.90, 30.38, 24.39, 16.14.

Procedure for the Synthesis of b2607e

### 1. Synthesis of Compound-31

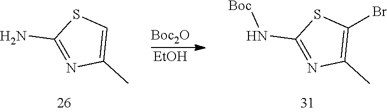

### tert-butyl N-(4-methyl-1,3-thiazol-2-yl)carbamate

A solution of 2-thiazolamine, 4-methyl- (30.0 g, 262.8 mmol, 1.0 equiv.) in ethyl alcohol (300 mL) was treated with di-tert-butyl dicarbonate (63.1 g, 289.0 mmol, 1.1 equiv.) at 25° C. The resulting mixture was stirred at 25° C. for 16 hours. The resulting mixture was concentrated under reduced pressure. The residue was purified by silica gel column chromatography, eluted with PE/EA (5/1) to afford tert-butyl N-(4-methyl-1,3-thiazol-2-yl)carbamate (30.0 g, 53.31% yield) as a white solid.

LCMS (ESI): [M+H]⁺=215.10.

¹H NMR (400 MHz, Chloroform-d) δ 6.44 (q, J=1.1 Hz, 1H), 2.35 (s, 3H), 1.53 (s, 9H).

### 2. Synthesis of Compound-32

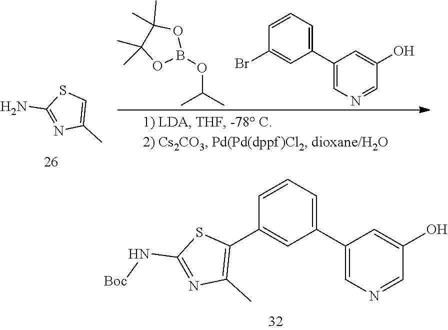

### tert-butyl N-[4-methyl-5-(4,4,5,5-tetramethyl-1,3,2-dioxaborolan-2-yl)-1,3-thiazol-2-yl]carbamate

A solution of tert-butyl N-(4-methyl-1,3-thiazol-2-yl)carbamate (15.0 g, 70.0 mmol, 1.0 equiv.) in THF (100 mL) was treated with LDA (2.0 M solution in THF) (140 mL, 280.0 mmol, 4.0 equiv.) at −78° C. for 30 minutes under nitrogen atmosphere. The resulting mixture was stirred at −78° C. for 2 hours under nitrogen atmosphere. To the above mixture was added 2-isopropoxy-4,4,5,5-tetramethyl-1,3,2-dioxaborolane (solution in 50 mL THF) (26.0 g, 140.0 mmol, 2.0 equiv.) dropwise over 30 minutes at −78° C. The resulting mixture was stirred at −78° C. for additional 2 hours. The resulting mixture was warmed to 25° C. The reaction mixture was poured into water/ice at 0° C. The resulting mixture was extracted with EtOAc (3×200 mL). The combined organic layers were washed with brine (200 mL), dried over anhydrous Na₂SO₄. After filtration, the filtrate was concentrated under reduced pressure. To a stirred mixture of tert-butyl N-[4-methyl-5-(4,4,5,5-tetramethyl-1,3,2-dioxaborolan-2-yl)-1,3-thiazol-2-yl]carbamate (1646 mg, 2.4 mmol, 1.1 equiv.) and 5-(3-bromophenyl)pyridin-3-ol (550 mg, 2.2 mmol, 1.0 equiv.) in 1,4-dioxane (10 mL) and H₂O (1 mL) was added Pd(dppf)Cl₂CH₂Cl₂ (180 mg, 0.22 mmol, 0.1 equiv.) in one portion at 25° C. under nitrogen atmosphere. The mixture was heated to 90° C. and stirred at 90° C. for 12 hours. The mixture was cooled to 25° C. The resulting mixture was concentrated under vacuum. The residue was purified by silica gel column chromatography, eluted with PE/EA (3/7) to afford tert-butyl N-(5-[3-(5-hydroxypyridin-3-yl)phenyl]-4-methyl-1,3-thiazol-2-ylcarbamate (510 mg, 60.51% yield) as a white solid.

LCMS (ESI): [M−H]-=382.15.

¹H NMR (400 MHz, DMSO-d₆) δ 11.46 (s, 1H), 10.07 (s, 1H), 8.38 (d, J=1.9 Hz, 1H), 8.16 (d, J=2.7 Hz, 1H), 7.65 (t, J=1.7 Hz, 1H), 7.60 (d, J=1.6 Hz, 1H), 7.55 (s, 1H), 7.49 (t, J=1.8 Hz, 1H), 7.42 (t, J=2.3 Hz, 1H), 2.36 (s, 3H), 1.49 (s, 9H).

3. Synthesis of b2607e

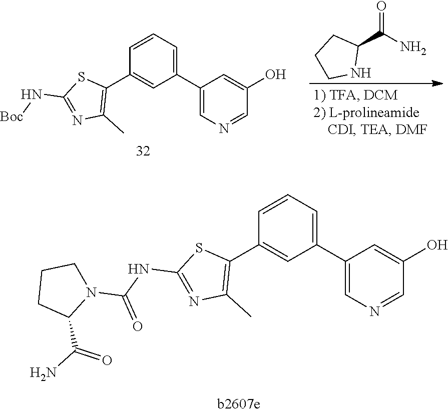 [SMILES: Cc1nc(NC(=O)OC(C)(C)C)sc1-c1cccc(-c2cncc(O)c2)c1 ; NC(=O)[C@@H]1CCCN1 ; Cc1nc(NC(=O)N2CCC[C@H]2C(N)=O)sc1-c1cccc(-c2cncc(O)c2)c1]

### 5-(3-(2-amino-4-methylthiazol-5-yl)phenyl)pyridin-3-ol

A solution of tert-butyl N-(5-[2-(4-fluorophenyl)pyrimidin-5-yl]-4-methyl-1,3-thiazol-2-ylcarbamate (480 mg, 1.25 mmol, 1.0 equiv.) and trifluoroacetic acid (2 mL) in DCM (4 mL) was stirred at 25° C. for 1 hour. The resulting mixture was concentrated under reduced pressure to afford 5-(3-(2-amino-4-methylthiazol-5-yl)phenyl)pyridin-3-ol (400 mg, crude). To a stirred solution of 5-[3-(2-amino-4-methyl-1,3-thiazol-5-yl)phenyl]pyridin-3-ol (200 mg, 0.7 mmol, 1.0 equiv.) in DMF (4 mL) was added N,N-Carbonyldiimidazole (137 mg, 0.8 mmol, 1.2 equiv.) in one portion at 25° C. The resulting mixture was stirred at 25° C. for 30 minutes. To the above mixture was added prolinamide (97 mg, 0.8 mmol, 1.2 equiv.) in one portion at 25° C. The resulting mixture was stirred at 25° C. for additional 1 hour. The resulting mixture was purified directly by reversed-phase flash chromatography with the following conditions: column, C18 silica gel; mobile phase, MeCN in water (10 mmol/L NH4HCO3), 10% to 40% gradient in 20 min; detector, UV 254 nm to afford (2S)-N1-(5-[3-(5-hydroxypyridin-3-yl)phenyl]-4-methyl-1,3-thiazol-2-ylpyrrolidine-1,2-dicarboxamide (12.8 mg, 4.3% yield) as a white solid.

LCMS (ESI): [M+H]⁺=424.15.

1H NMR (400 MHz, DMSO-d6) δ 10.74 (s, 1H), 8.38 (s, 1H), 8.22-8.16 (m, 1H), 7.64-7.53 (m, 3H), 7.49-7.38 (m, 3H), 6.96 (s, 1H), 4.29 (s, 1H), 3.63-3.58 (m, 1H), 3.50-3.44 (m, 1H), 2.38 (s, 3H), 2.12-2.05 (m, 1H), 1.91-1.86 (m, 3H).

13C NMR (101 MHz, DMSO) δ 174.49, 164.19, 154.33, 138.99, 138.27, 137.71, 136.48, 133.86, 130.17, 128.33, 126.97, 125.88, 122.39, 120.51, 112.88, 60.36, 46.91, 30.46, 24.42, 16.23.

Procedure for the Synthesis of ecbde6

### 1. Synthesis of Compound-34

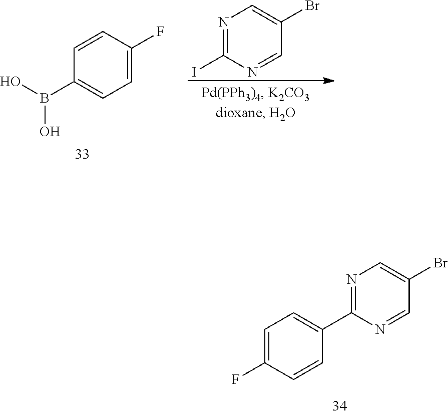 [SMILES: OB(O)c1ccc(F)cc1 ; Brc1cnc(I)nc1 ; Fc1ccc(-c2ncc(Br)cn2)cc1]

### 5-bromo-2-(4-fluorophenyl)pyrimidine

To a mixture of 4-fluorophenylboronic acid (5.0 g, 35.0 mmol, 1.0 equiv.) and 5-bromo-2-iodopyrimidine (8.2 g, 28.3 mmol, 0.8 equiv.) in dioxane (50 mL) were added Pd(PPh₃)₄ (4.1 g, 3.3 mmol, 0.1 equiv.) and K₂CO₃ (24.7 g, 178.4 mmol, 5.0 equiv.) in portions at 25° C. under nitrogen atmosphere. The resulting mixture was heated to 100° C. and stirred at 100° C. for 2 hours under nitrogen atmosphere. The resulting mixture was cooled to 25° C. The resulting mixture was filtered, the filter cake was washed with ethyl acetate (3×50 mL). The filtrate was concentrated under reduced pressure. The resulting mixture was extracted with EtOAc (2×100 mL). The combined organic layers were washed with brine (100 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (0.1% FA), 10% to 70% gradient in 30 min; detector, UV 254 nm. This resulted in 5-bromo-2-(4-fluorophenyl)pyrimidine (1.5 g, 17.03% yield) as an off-white solid.

LCMS (ESI): [M+H]⁺=253.10.

¹H NMR (400 MHz, DMSO-d₆) δ 9.08 (s, 2H), 8.46-8.36 (m, 2H), 7.45-7.32 (m, 2H).

### 2. Synthesis of Compound-35

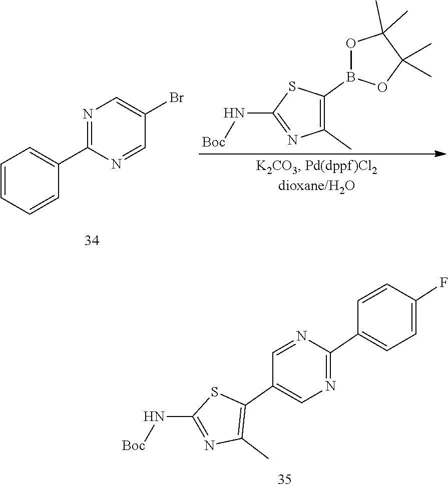 [SMILES: Brc1cnc(-c2ccccc2)nc1 ; Cc1nc(NC(=O)OC(C)(C)C)sc1B1OC(C)(C)C(C)(C)O1 ; Cc1nc(NC(=O)OC(C)(C)C)sc1-c1cnc(-c2ccc(F)cc2)nc1]

### tert-butyl N-(5-[2-(4-fluorophenyl)pyrimidin-5-yl]-4-methyl-1,3-triazol-2-ylcarbamate

To a stirred mixture of tert-butyl N-[4-methyl-5-(4,4,5,5-tetramethyl-1,3,2-dioxaborolan-2-yl)-1,3-thiazol-2-yl]carbamate (887 mg, 1.3 mmol, 1.1 equiv., 50%), Cs₂CO₃ (1159 mg, 3.6 mmol, 3.0 equiv.) and 5-bromo-2-(4-fluorophenyl)pyrimidine (300 mg, 1.2 mmol, 1.0 equiv.) in 1,4-dioxane (10 mL) and H₂O (1 mL) was added Pd(dppf)Cl₂CH₂Cl₂ (97 mg, 0.1 mmol, 0.1 equiv.) in one portion at 25° C. under nitrogen atmosphere. The mixture was heated to 90° C. and stirred at 90° C. for 12 hours. The mixture was cooled to 25° C. The resulting mixture was concentrated under vacuum. The residue was purified by silica gel column chromatography, eluted with PE/EA (25/75) to afford tert-butyl N-(5-[2-(4-fluorophenyl)pyrimidin-5-yl]-4-methyl-1,3-thiazol-2-ylcarbamate (220 mg, 48.01% yield) as a white solid.

LCMS (ESI): [M+H]⁺=387.20.

¹H NMR (400 MHz, DMSO-d₆) δ 11.64 (s, 1H), 9.00 (s, 2H), 8.51-8.43 (m, 2H), 7.43-7.34 (m, 2H), 2.39 (s, 3H), 1.51 (s, 9H).

3. Synthesis of ecbde6

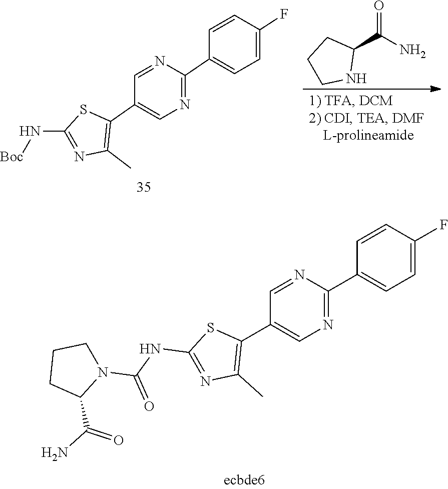 [SMILES: Cc1nc(NC(=O)OC(C)(C)C)sc1-c1cnc(-c2ccc(F)cc2)nc1 ; NC(=O)[C@@H]1CCCN1 ; Cc1nc(NC(=O)N2CCC[C@H]2C(N)=O)sc1-c1cnc(-c2ccc(F)cc2)nc1]

### (2S)-N1-(5-[2-(4-fluorophenyl)pyrimidin-5-yl]-4-methyl-1,3-thiazol-2-ylpyrrolidine-1,2-dicarboxamide

A solution of tert-butyl N-(5-[2-(4-fluorophenyl)pyrimidin-5-yl]-4-methyl-1,3-thiazol-2-ylcarbamate (220 mg, 0.57 mmol, 1.0 equiv.) and trifluoroacetic acid (1 mL) in DCM (2 mL) was stirred at 25° C. for 1 hour. The resulting mixture was concentrated under reduced pressure to afford 5-(2-(4-fluorophenyl)pyrimidin-5-yl)-4-methylthiazol-2-amine (180 mg, crude). To a stirred solution of 5-[2-(4-fluorophenyl)pyrimidin-5-yl]-4-methyl-1,3-thiazol-2-amine (170 mg, 0.6 mmol, 1.0 equiv.) and Et₃N (180 mg, 1.8 mmol, 3.0 equiv.) in DMF (4 mL) was added N,N-Carbonyldiimidazole (116 mg, 0.7 mmol, 1.2 equiv.) in one portion at 25° C. The resulting mixture was stirred at 25° C. for 30 minutes. To the above mixture was added prolinamide (81 mg, 0.7 mmol, 1.2 equiv.) in one portion at 25° C. The resulting mixture was stirred at 25° C. for additional 1 hour. The resulting mixture was purified directly by reversed-phase flash chromatography with the following conditions: column, C18 silica gel; mobile phase, MeCN in water (10 mmol/L NH₄HCO₃), 10% to 40% gradient in 20 min; detector, UV 254 nm to afford (2S)-N1-(5-[2-(4-fluorophenyl)pyrimidin-5-yl]-4-methyl-1,3-thiazol-2-ylpyrrolidine-1,2-dicarboxamide (15.0 mg, 5.9% yield) as a white solid.

LCMS (ESI): [M+H]⁺=427.15.

¹H NMR (400 MHz, DMSO-d₆) δ 11.00 (s, 1H), 8.97 (s, 2H), 8.47-8.44 (m, 2H), 7.39-7.34 (m, 3H), 6.95 (s, 1H), 4.29 (s, 1H), 3.64-3.58 (m, 1H), 3.50-3.44 (m, 1H), 2.41 (s, 3H), 2.12-2.07 (m, 1H), 1.90-1.83 (m, 3H).

¹³C NMR (101 MHz, DMSO) δ 174.37, 165.66, 163.20, 160.57, 156.26, 133.80, 133.77, 130.46, 130.37, 125.75, 116.35, 116.13, 60.41, 46.93, 30.42, 24.46, 16.31.

Procedure for the Synthesis of b0656f

### 1. Synthesis of Compound-37

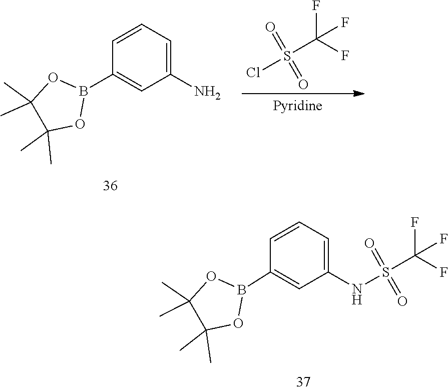 [SMILES: CC1(C)OB(c2cccc(N)c2)OC1(C)C ; O=S(=O)(Cl)C(F)(F)F ; CC1(C)OB(c2cccc(NS(=O)(=O)C(F)(F)F)c2)OC1(C)C]

### 1,1,1-trifluoro-N-(3-(4,4,5,5-tetramethyl-1,3,2-dioxaborolan-2-yl)phenyl)methanesulfonamide

A solution of 3-(4,4,5,5-tetramethyl-1,3,2-dioxaborolan-2-yl)aniline (5.0 g, 22.8 mmol, 1.0 equiv.) and trifluoromethanesulfonyl chloride (8.0 g, 45.6 mmol, 2.0 equiv.) in pyridine (50 mL) was stirred for 5 hours at 25° C. under nitrogen atmosphere. The resulting mixture was concentrated under reduced pressure. The residue was purified by silica gel column chromatography, eluted with CH2Cl2/MeOH (10:1) to afford 1,1,1-trifluoro-N-(3-(4,4,5,5-tetramethyl-1,3,2-dioxaborolan-2-yl)phenyl)methanesulfonamide (3.0 g, 37.4% yield) as a brown oil.

LCMS (ESI): [M−H]⁻=268.10.

1H NMR (400 MHz, DMSO-d6) δ11.82 (s, 1H), 7.57-7.53 (m, 2H), 7.46-7.37 (m, 2H), 1.30 (s, 12H).

2. Synthesis of b0656f

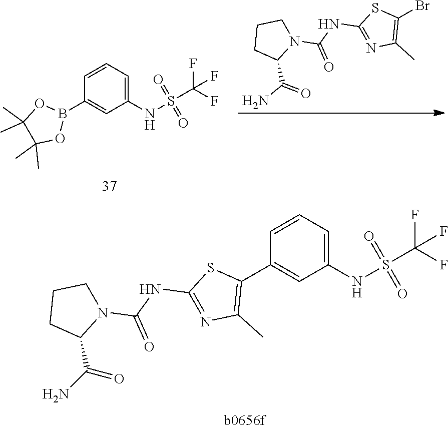 [SMILES: CC1(C)OB(c2cccc(NS(=O)(=O)C(F)(F)F)c2)OC1(C)C ; Cc1nc(NC(=O)N2CCC[C@H]2C(N)=O)sc1Br ; Cc1nc(NC(=O)N2CCC[C@H]2C(N)=O)sc1-c1cccc(NS(=O)(=O)C(F)(F)F)c1]

### (S)-N1-(4-methyl-5-(3-((trifluoromethyl)sulfonamido)phenyl)thiazol-2-yl)pyrrolidine-1,2-dicarboxamide

A solution of 1,1,1-trifluoro-N-[3-(4,4,5,5-tetramethyl-1,3,2-dioxaborolan-2-yl)phenyl]methanesulfonamide (300 mg, 0.9 mmol, 1.0 equiv.) in dioxane (3 mL) and H₂O (0.3 mL) was treated with (2S)-N1-(5-bromo-4-methyl-1,3-thiazol-2-yl)pyrrolidine-1,2-dicarboxamide (285 mg, 0.9 mmol, 1.0 equiv.) and K₂CO₃ (354 mg, 2.6 mmol, 3.0 equiv.) at 25° C. under nitrogen atmosphere followed by the addition of Pd(dppf)Cl₂ (63 mg, 0.1 mmol, 0.1 equiv.) in portions at 25° C. The reaction mixture was heated to 90° C. The resulting mixture was stirred for 2 hours at 90° C. under nitrogen atmosphere. The resulting mixture was slowly cooled to 25° C. After filtration, the filter cake was washed with MeOH (2×5 mL). The combined filtrates were concentrated under reduced pressure. The crude product was purified by prep-HPLC with the following conditions (Column: XBridge BEH C185 μm, 19*250 mm; Mobile Phase A: water (10 mmol/L NH₄HCO₃+0.1% NH₃·H₂O), Mobile Phase B: ACN; Flow rate: 25 mL/min; Gradient: 18% B to 30% B in 8 min; Wave Length: 254 nm/220 nm; RT1(min): 6.47) to afford (S)-N1-(4-methyl-5-(3-((trifluoromethyl)sulfonamido)phenyl)thiazol-2-yl)pyrrolidine-1,2-dicarboxamide (10.2 mg, 2.4% yield) as a white solid.

LCMS (ESI): [M+H]⁺=478.05.

¹H NMR (400 MHz, DMSO-d₆) δ 10.86 (s, 1H), 7.37-7.32 (m, 2H), 7.22-7.21 (m, 1H), 7.15-7.07 (m, 2H), 6.97-6.95 (m, 1H), 4.30 (s, 1H), 3.63-3.57 (m, 1H), 3.48-3.42 (m, 1H), 2.32 (s, 3H), 2.11-2.04 (m, 1H), 1.90-1.85 (m, 3H).

¹³C NMR (101 MHz, DMSO) δ 174.58, 159.34, 153.84, 141.50, 138.85, 133.90, 130.24, 125.65, 125.01, 122.50, 121.47, 119.18, 60.35, 46.92, 30.45, 24.40, 16.07.

Procedure for the Synthesis of f9De5d

### 1. Synthesis of Compound-39

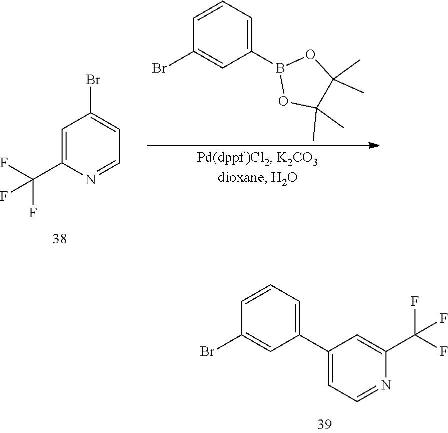 [SMILES: FC(F)(F)c1cc(Br)ccn1 ; CC1(C)OB(c2cccc(Br)c2)OC1(C)C ; FC(F)(F)c1cc(-c2cccc(Br)c2)ccn1]

### 4-(3-bromophenyl)-2-(trifluoromethyl)pyridine

A solution of 4-bromo-2-(trifluoromethyl)pyridine (1.0 g, 4.4 mmol, 1.0 equiv.) in dioxane (9 mL) and H2O (1 mL) was treated with K2CO3 (1.8 g, 13.3 mmol, 3.0 equiv.) at 25° C. followed by the addition of Pd(dppf)C12 (324 mg, 0.4 mmol, 0.1 equiv.) in portions at 25° C. under nitrogen atmosphere. The reaction mixture was heated to 80° C. The resulting mixture was stirred for 2 hours at 80° C. under nitrogen atmosphere. The resulting mixture was cooled to 25° C. The resulting mixture was filtered, the filter cake was washed with MeOH (3×5 mL). The combined filtrates were concentrated under reduced pressure. The residue was purified by silica gel column chromatography, eluted with PE/EA (7/1) to afford 4-(3-bromophenyl)-2-(trifluoromethyl)pyridine (1.0 g, 74.82% yield) as a colorless oil.

LCMS (ESI): [M+H]+=302.00.

1H NMR (400 MHz, DMSO-d6) δ 8.84 (d, J=5.1 Hz, 1H), 8.25 (d, J=1.7 Hz, 1H), 8.17 (t, J=1.9 Hz, 1H), 8.09 (dd, J=5.2, 1.8 Hz, 1H), 7.94 (ddd, J=7.8, 1.8, 1.0 Hz, 1H), 7.73 (ddd, J=8.0, 2.0, 0.9 Hz, 1H), 7.52 (t, J=7.9 Hz, 1H).

### 2. Synthesis of Compound-40

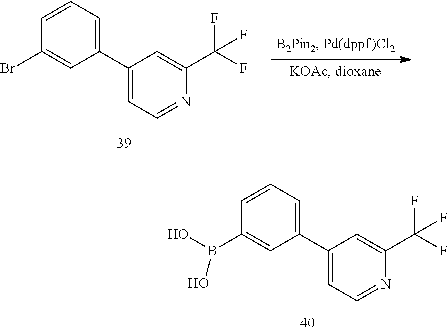 [SMILES: FC(F)(F)c1cc(-c2cccc(Br)c2)ccn1 ; OB(O)c1cccc(-c2ccnc(C(F)(F)F)c2)c1]

### (3-(2-(trifluoromethyl)pyridin-4-yl)phenyl)boronic acid

A solution of 4-(3-bromophenyl)-2-(trifluoromethyl)pyridine (880 mg, 2.9 mmol, 1.0 equiv.) in dioxane (6 mL) was treated with bis(pinacolato)diboron (1.5 g, 5.8 mmol, 2.0 equiv.) and AcOK (858 mg, 8.7 mmol, 3.0 equiv.) at 25° C. under nitrogen atmosphere followed by the addition of Pd(dppf)Cl₂ (213 mg, 0.3 mmol, 0.1 equiv.) in one portions at 25° C. under nitrogen atmosphere. The reaction mixture was heated to 100° C. The resulting mixture was stirred at 100° C. for 16 hours under nitrogen atmosphere. The resulting mixture was cooled to 25° C. and concentrated under reduced pressure. The residue was purified by reversed-phase flash chromatography with the following conditions: column, C18 silica gel; mobile phase, MeCN in water (0.1% FA), 10% to 50% gradient in 10 min; detector, UV 254 nm. This resulted in (3-(2-(trifluoromethyl)pyridin-4-yl)phenyl)boronic acid (350 mg, 34.41% yield) as a white solid.

LCMS (ESI): [M−H]-=265.90.

¹H NMR (400 MHz, DMSO-d₆) δ 8.84 (t, J=5.5 Hz, 1H), 8.32 (s, 1H), 8.28 (s, 1H), 8.22-8.15 (m, 1H), 8.07 (dt, J=5.7, 2.8 Hz, 1H), 7.95 (ddt, J=18.9, 7.4, 1.2 Hz, 1H), 7.57 (dt, J=22.6, 7.5 Hz, 1H).

3. Synthesis of f9de5d

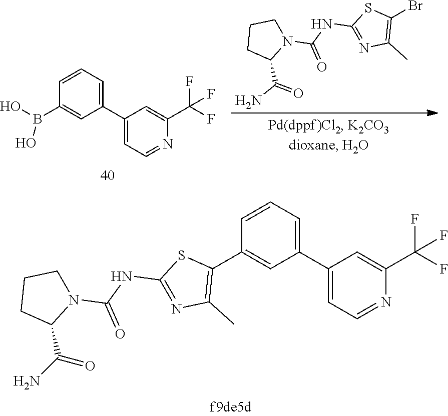 [SMILES: OB(O)c1cccc(-c2ccnc(C(F)(F)F)c2)c1 ; Cc1nc(NC(=O)N2CCC[C@H]2C(N)=O)sc1Br ; Cc1nc(NC(=O)N2CCC[C@H]2C(N)=O)sc1-c1cccc(-c2ccnc(C(F)(F)F)c2)c1]

### (S)-N1-(4-methyl-5-(3-(2-(trifluoromethyl)pyridin-4-yl)phenyl)thiazol-2-yl)pyrrolidine-1,2-dicarboxamide

A solution of 3-[2-(trifluoromethyl)pyridin-4-yl]phenylboronic acid (150 mg, 0.6 mmol, 1.0 equiv.) in dioxane (3.2 mL) and H₂O (0.8 mL) was treated with (2S)-N1-(5-bromo-4-methyl-1,3-thiazol-2-yl)pyrrolidine-1,2-dicarboxamide (187 mg, 0.6 mmol, 1.0 equiv.) and K₂CO₃ (233 mg, 1.7 mmol, 3.0 equiv.) at 25° C. under nitrogen atmosphere followed by the addition of Pd(dppf)Cl₂ (41 mg, 0.1 mmol, 0.1 equiv.) in one portion at 25° C. The reaction mixture was heated to 90° C. The resulting mixture was stirred at 90° C. for 16 hours. The resulting mixture was slowly cooled to 25° C. The resulting mixture was filtered, the filter cake was washed with MeOH (3×5 mL). The filtrates were concentrated under reduced pressure. The crude product was purified by prep-HPLC with the following conditions (Column: XBridge BEH C185 μm, 19*250 mm; Mobile Phase A: water (10 mmol/L NH₄HCO₃+0.1% NH₃·H₂O), Mobile Phase B: ACN; Flow rate: 25 mL/min; Gradient: 35% B to 45% B in 10 min; Wave Length: 254 nm/220 nm; RT1(min): 9.53) to afford (S)-N1-(4-methyl-5-(3-(2-(trifluoromethyl)pyridin-4-yl)phenyl)thiazol-2-yl)pyrrolidine-1,2-dicarboxamide (10.7 mg, 3.84% yield) as a white solid.

LCMS (ESI): [M+H]⁺=476.15.

¹H NMR (400 MHz, DMSO-d₆) δ 10.82 (s, 1H), 8.84 (s, 1H), 8.24 (s, 1H), 8.17-8.04 (m, 1H), 7.95-7.89 (m, 1H), 7.89-7.7 (m, 1H), 7.67-7.55 (m, 2H), 7.38 (s, 1H), 6.95 (s, 1H), 4.30 (s, 1H), 3.61 (s, 1H), 3.53-3.41 (m, 1H), 2.38 (s, 3H), 2.20-2.00 (m, 1H), 1.98-1.71 (m, 3H).

¹³C NMR (101 MHz, DMSO) δ 174.46, 151.29, 149.46, 147.99, 147.66, 137.12, 134.19, 130.36, 130.24, 127.42, 126.33, 125.38, 123.57, 120.85, 118.84, 60.34, 46.90, 30.43, 24.42, 16.23.

### Procedure for the Synthesis of 03Adde

### 1. Synthesis of Compound-42

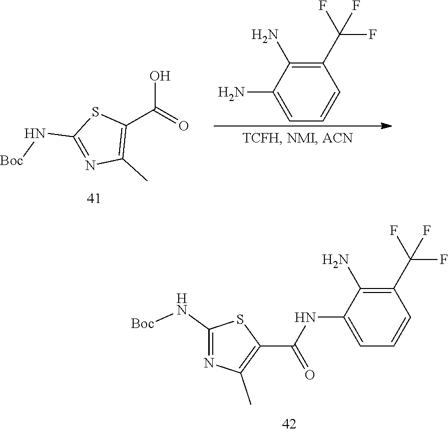 [SMILES: Cc1nc(NC(=O)OC(C)(C)C)sc1C(=O)O ; Nc1cccc(C(F)(F)F)c1N ; Cc1nc(NC(=O)OC(C)(C)C)sc1C(=O)Nc1cccc(C(F)(F)F)c1N]

### tert-butyl N-(5-([2-amino-3-(trifluoromethyl)phenyl]carbamoyl-4-methyl-1,3-thiazol-2-yl)carbamate

A solution of 2-[(tert-butoxycarbonyl)amino]-4-methyl-1,3-thiazole-5-carboxylic acid (1.0 g, 3.9 mmol, 1.0 equiv.) and 3-(trifluoromethyl)benzene-1,2-diamine (682 mg, 3.9 mmol, 1.0 equiv.) in ACN (10 mL) was treated with NMI (795 mg, 9.7 mmol, 2.5 equiv.) at 25° C. for 5 minutes under nitrogen atmosphere followed by the addition of TCFH (1.6 g, 5.8 mmol, 1.5 equiv.) in portions at 25° C. The resulting mixture was stirred at 25° C. for 2 hours under nitrogen atmosphere. The resulting mixture was concentrated under reduced pressure. The residue was purified by silica gel column chromatography, eluted with PE/EA (9/1) to afford tert-butyl N-(5-([2-amino-3-(trifluoromethyl)phenyl]carbamoyl-4-methyl-1,3-thiazol-2-yl)carbamate (800 mg, 49.61% yield) as a reddish brown solid.

LCMS (ESI): [M+H]⁺=417.10.

¹H NMR (400 MHz, Chloroform-d) δ 7.51-7.48 (m, 1H), 7.41-7.39 (m, 1H), 7.27 (s, 1H), 6.88-6.84 (m, 1H), 2.69 (s, 3H), 1.56 (s, 9H).

### 2. Synthesis of Compound-43

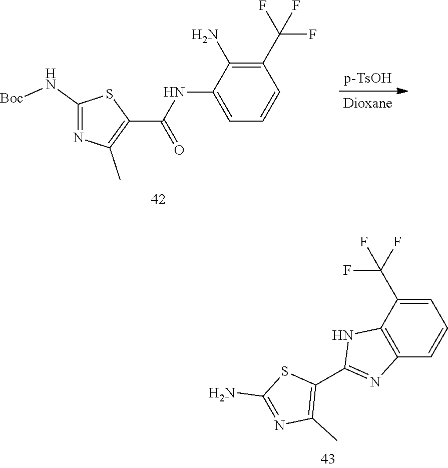 [SMILES: Cc1nc(NC(=O)OC(C)(C)C)sc1C(=O)Nc1cccc(C(F)(F)F)c1N ; Cc1nc(N)sc1-c1nc2cccc(C(F)(F)F)c2[nH]1]

### 4-methyl-5-[4-(trifluoromethyl)-3H-1,3-benzodiazol-2-yl]-1,3-thiazol-2-amine

A solution tert-butyl N-(5-([2-amino-3-(trifluoromethyl)phenyl]carbamoyl-4-methyl-1,3-thiazol-2-yl)carbamate (700 mg, 1.7 mmol, 1.0 equiv.) and 4-methylbenzene-1-sulfonic acid hydrate (959 mg, 5.0 mmol, 3.0 equiv.) in dioxane (7 mL) was stirred at 110° C. for 1 hour under nitrogen atmosphere. The mixture was cooled to 25° C. The resulting mixture was concentrated under reduced pressure. The residue was purified by silica gel column chromatography, eluted with PE/EA (9/1) to afford 4-methyl-5-[4-(trifluoromethyl)-3H-1,3-benzodiazol-2-yl]-1,3-thiazol-2-amine (620 mg, 30.25% yield) as a brown solid.

LCMS (ESI): [M+H]⁺=299.05.

¹H NMR (400 MHz, Chloroform-d) δ 9.57 (s, 1H), 7.65-7.50 (m, 3H), 7.07 (d, J=7.3 Hz, 2H), 2.26 (s, 3H).

3. Synthesis of 03adde

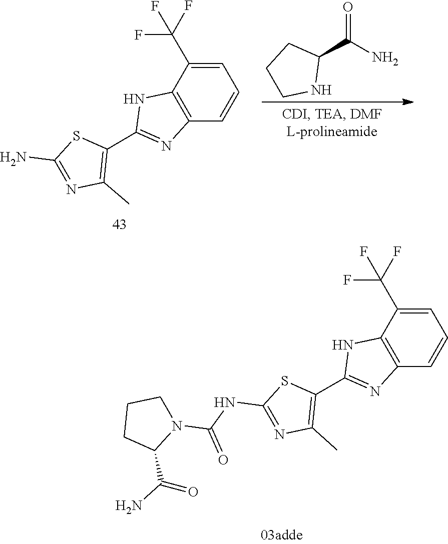

### (2S)-NJ-(4-methyl-5-[4-(trifluoromethyl)-3H-1,3-benzodiazol-2-yl]-1,3-thiazol-2-ylpyrroidine-1,2-dicarboxamide

A solution of 4-methyl-5-[4-(trifluoromethyl)-3H-1,3-benzodiazol-2-yl]-1,3-thiazol-2-amine (100 mg, 0.3 mmol, 1.0 equiv.) in DMF (1 mL) was treated with TEA (102 mg, 1.0 mmol, 3.0 equiv.) at 25° C. followed by the addition of 1-[(1H-imidazol-1-yl)carbonyl]-1H-imidazole (82 mg, 0.5 mmol, 1.5 equiv.) in portions at 25° C. The resulting mixture was heated to 70° C. and stirred at 70° C. for 2 hours. The reaction mixture was cooled to 25° C. To the above mixture was added prolinamide (57 mg, 0.5 mmol, 1.5 equiv.) in one portion at 25° C. The resulting mixture was stirred at 25° C. for additional 1 hour. The resulting mixture was extracted with CHCl₃ (3×50 mL). The combined organic layers were washed with brine (3×50 mL), dried over anhydrous Na₂SO₄. After filtration, the filtrate was concentrated under reduced pressure. The residue was purified by silica gel column chromatography, eluted with CH₂Cl₂/MeOH (10/1) to afford (2S)-N1-(4-methyl-5-[4-(trifluoromethyl)-3H-1,3-benzodiazol-2-yl]-1,3-thiazol-2-ylpyrrolidine-1,2-dicarboxamide (10.9 mg, 7.43% yield) as a white solid.

LCMS (ESI): [M+H]⁺=439.05.

¹H NMR (400 MHz, DMSO-d₆) δ 11.99 (s, 2H), 7.76 (d, J=8.0 Hz, 1H), 7.49 (d, J=7.6 Hz, 1H), 7.45-7.41 (m, 1H), 7.31 (t, J=7.8 Hz, 1H), 6.98 (s, 1H), 4.32 (s, 1H), 3.64-3.59 (m, 1H), 3.54-3.43 (m, 1H), 2.68 (s, 3H), 2.13-2.06 (m, 1H), 1.91-1.86 (m, 3H).

¹³C NMR (101 MHz, DMSO) δ 174.39, 165.67, 163.20, 160.58, 156.26, 133.80, 133.77, 130.46, 130.37, 125.75, 121.86, 116.35, 116.13, 60.42, 46.93, 30.43, 24.45, 16.30.

### Procedure for the Synthesis of 55675a

### 1. Synthesis of Compound-45

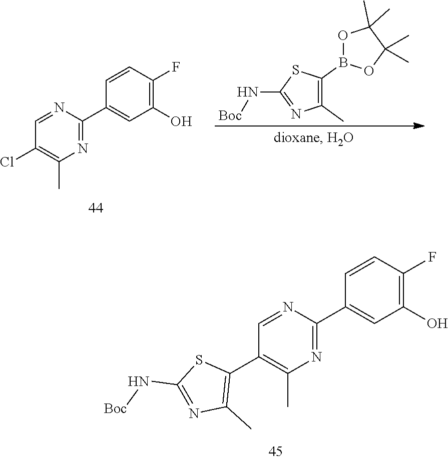 [SMILES: Cc1nc(-c2ccc(F)c(O)c2)ncc1Cl ; Cc1nc(NC(=O)OC(C)(C)C)sc1B1OC(C)(C)C(C)(C)O1 ; Cc1nc(-c2ccc(F)c(O)c2)ncc1-c1sc(NC(=O)OC(C)(C)C)nc1C]

### N-(5-[2-(4-fluoro-3-hydroxyphenyl)-4-methylpyrimidin-5-yl]-4-methyl-1,3-thiazol-2-ylcarbamate

A mixture of tert-butyl N-[4-methyl-5-(4,4,5,5-tetramethyl-1,3,2-dioxaborolan-2-yl)-1,3-thiazol-2-yl]carbamate (1021 mg, 3.0 mmol, 3.0 equiv.), 5-(5-chloro-4-methylpyrimidin-2-yl)-2-fluorophenol (239 mg, 1.0 mmol, 1.0 equiv.), Xphos Pd G3 (85 mg, 0.1 mmol, 0.1 equiv.), Xphos (95 mg, 0.2 mmol, 0.2 equiv.) and Cs2CO3 (977 mg, 3.0 mmol, 3.0 equiv.) in dioxane (1 mL) and H2O (0.1 mL) was stirred at 100° C. for 1 hour under nitrogen atmosphere. The mixture was allowed to cool down to 25° C. After filtration, the filter cake was washed with MeOH (3×10 mL). The combined filtrates were concentrated under reduced pressure. The residue was purified by reversed-phase flash chromatography with the following conditions: column, C18 silica gel; mobile phase, MeCN in water (10 mmol/L NH4HCO3), 10% to 50% gradient in 10 min; detector, UV 254 nm. This resulted in tert-butyl N-(5-[2-(4-fluoro-3-hydroxyphenyl)-4-methylpyrimidin-5-yl]-4-methyl-1,3-thiazol-2-ylcarbamate (60 mg, 14.43% yield) as a white solid.

LCMS (ESI): [M+H]+=417.15.

1H NMR (400 MHz, Chloroform-d) δ 8.57 (s, 1H), 8.20-8.17 (m, 1H), 8.07-8.03 (m, 1H), 7.22-7.17 (m, 1H), 2.54 (s, 3H), 2.24 (s, 3H), 2.01 (s, 1H), 1.56 (s, 8H).

### 2. Synthesis of Compound-46

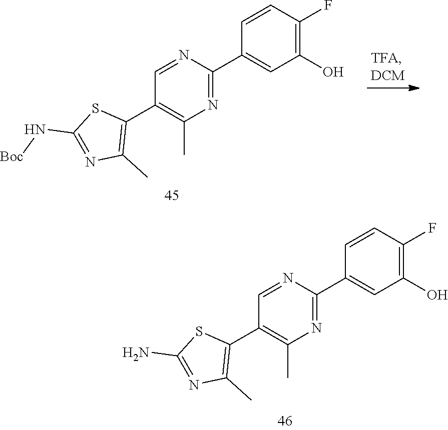 [SMILES: Cc1nc(-c2ccc(F)c(O)c2)ncc1-c1sc(NC(=O)OC(C)(C)C)nc1C ; Cc1nc(-c2ccc(F)c(O)c2)ncc1-c1sc(N)nc1C]

### 5-[5-(2-amino-4-methyl-4,3-thiazol-5-yl)-4-methylpyrimidin-2-yl]-2-fluorophenol

A solution of tert-butyl N-(5-[2-(4-fluoro-3-hydroxyphenyl)-4-methylpyrimidin-5-yl]-4-methyl-1,3-thiazol-2-ylcarbamate (55 mg, 0.1 mmol, 1.0 equiv.) and TFA (0.2 mL) in DCM (1 mL) was stirred at 25° C. for 1 hour under nitrogen atmosphere. The resulting mixture was concentrated under reduced pressure. The residue was purified by prep-TLC (PE/EA 10/1) to afford 5-[5-(2-amino-4-methyl-1,3-thiazol-5-yl)-4-methylpyrimidin-2-yl]-2-fluorophenol (43 mg, crude) as a white solid.

LCMS (ESI): [M+H]⁺=317.10.

¹H NMR (400 MHz, Chloroform-d) δ 8.57 (s, 1H), 8.18-8.15 (m, 1H), 8.05-8.01 (m, 1H), 7.20-7.16 (m, 1H), 5.16 (s, 2H), 2.54 (s, 3H), 2.12 (s, 3H).

### 3. Synthesis of 55675a

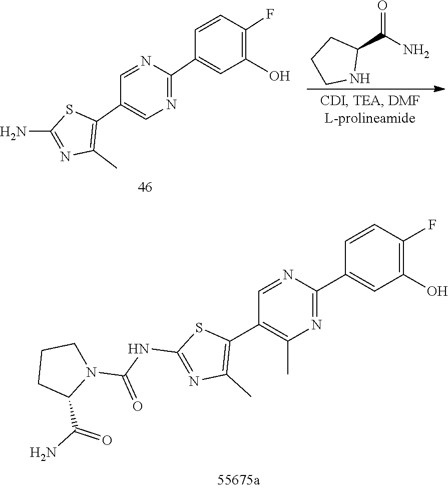

### (2S)-N1-{5-[2-(4-fluoro-3-hydroxyphenyl)-4-methylpyrimidin-5-yl]-4-methyl-1,3-thiazol-2-yl}pyrrolidine-1,2-dicarboxamide

A solution of 5-[5-(2-amino-4-methyl-1,3-thiazol-5-yl)-4-methylpyrimidin-2-yl]-2-fluorophenol (38 mg, 0.1 mmol, 1.0 equiv.), TEA (36 mg, 0.4 mmol, 3.0 equiv.) and 1-[(1H-imidazol-1-yl)carbonyl]-1H-imidazole (29 mg, 0.2 mmol, 1.5 equiv.) in DMF (1 mL) was stirred at 70° C. for 2 hours under nitrogen atmosphere. The mixture was allowed to cool down to 25° C. To the above mixture was added prolinamide (13.7 mg, 0.1 mmol, 1.0 equiv.) at 25° C. The resulting mixture was stirred at 25° C. for additional 1 hour. The residue was purified directly by reversed-phase flash chromatography with the following conditions: column, C18 silica gel; mobile phase, MeCN in water (10 mmol/L NH4HCO3), 10% to 50% gradient in 10 min; detector, UV 254 nm. This resulted in (2S)-N1-{5-[2-(4-fluoro-3-hydroxyphenyl)-4-methylpyrimidin-5-yl]-4-methyl-1,3-thiazol-2-yl}pyrrolidine-1,2-dicarboxamide (20.5 mg, 37.41% yield) as a white solid.

LCMS (ESI): [M+H]⁺=457.00.

1H NMR (400 MHz, DMSO-d6) δ 10.80-10.24 (m, 2H), 8.68 (s, 1H), 8.10-8.08 (m, 1H), 7.90-7.87 (m, 1H), 7.39 (s, 1H), 7.29-7.24 (m, 1H), 6.97 (s, 1H), 4.30 (s, 1H), 3.62 (s, 1H), 3.50-3.44 (m, 1H), 2.48 (s, 3H), 2.13-2.08 (m, 4H), 1.88 (d, J=6.5 Hz, 3H).

13C NMR (101 MHz, DMSO) δ 174.44, 166.49, 161.75, 158.77, 154.66, 152.22, 145.61, 145.49, 134.03, 134.00, 123.84, 119.85, 119.78, 117.57, 116.91, 116.72, 60.35, 46.94, 30.45, 24.41, 23.38, 15.65.

Procedure for the Synthesis of b7a4b5

### 1. Synthesis of Compound-48

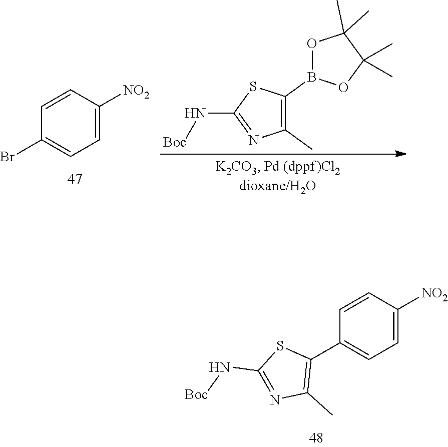 [SMILES: O=[N+]([O-])c1ccc(Br)cc1 ; Cc1nc(NC(=O)OC(C)(C)C)sc1B1OC(C)(C)C(C)(C)O1 ; Cc1nc(NC(=O)OC(C)(C)C)sc1-c1ccc([N+](=O)[O-])cc1]

### tert-butyl N-[4-methyl-5-(4-nitrophenyl)-1,3-thiazol-2-yl]carbamate

To a stirred mixture of tert-butyl N-[4-methyl-5-(4,4,5,5-tetramethyl-1,3,2-dioxaborolan-2-yl)-1,3-thiazol-2-yl]carbamate (7.4 g, 10.9 mol, 2.2 equiv. 50%), Cs₂CO₃ (4.8 g, 14.9 mmol, 3.0 equiv.) and 4-bromo-1-nitrobenzene (1.0 g, 5.0 mmol, 1.0 equiv.) in 1,4-dioxane (10 mL) and H₂O (1 mL) was added Pd(dppf)Cl₂CH₂Cl₂ (404 mg, 0.5 mmol, 0.1 equiv.) in one portion at 25° C. under nitrogen atmosphere. The mixture was heated to 90° C. and stirred at 90° C. for 12 hours. The mixture was cooled to 25° C. The resulting mixture was concentrated under vacuum. The residue was purified by silica gel column chromatography, eluted with PE/EA (5/2) to afford tert-butyl N-[4-methyl-5-(4-nitrophenyl)-1,3-thiazol-2-yl]carbamate (1.1 g, 66.31% yield) as a yellow solid.

LCMS (ESI): [M+H]⁺=336.15.

¹H NMR (400 MHz, DMSO-d₆) δ 11.65 (s, 1H), 8.26 (d, J=8.8 Hz, 2H), 7.73-7.71 (m, 2H), 2.40 (s, 3H), 1.50 (s, 9H).

### 2. Synthesis of Compound-49

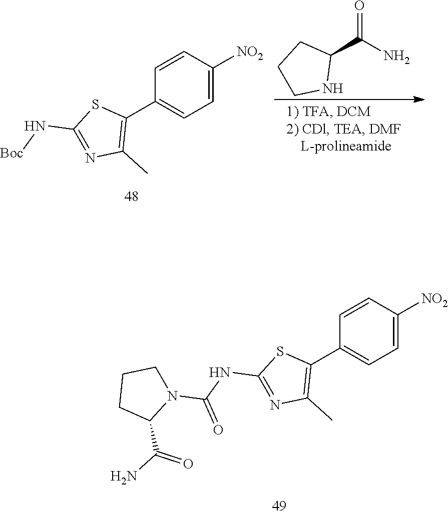

### (2S)-N1-[4-methyl-5-(4-nitrophenyl)-1,3-thiazol-2-yl]pyrrolidine-1,2-dicarboxamide

A solution of tert-butyl N-(5-[2-(4-fluorophenyl)pyrimidin-5-yl]-4-methyl-1,3-thiazol-2-ylcarbamate (220 mg, 0.57 mmol, 1.0 equiv.) and trifluoroacetic acid (1 mL) in DCM (2 mL) was stirred at 25° C. for 1 hour. The resulting mixture was concentrated under reduced pressure to afford the 4-methyl-5-(4-nitrophenyl)-1,3-thiazol-2-amine (600 mg, crude). To a stirred solution of 4-methyl-5-(4-nitrophenyl)-1,3-thiazol-2-amine (600 mg, 2.6 mmol, 1.0 equiv.) and Et₃N (774 mg, 7.7 mmol, 3.0 equiv.) in DMF (10 mL) was added N,N-Carbonyldiimidazole (496 mg, 3.1 mmol, 1.2 equiv.) in one portion at 25° C. The resulting mixture was stirred at 25° C. for 30 min. To the above mixture was added prolinamide (349 mg, 3.1 mmol, 1.2 equiv.) in one portion at 25° C. The resulting mixture was stirred at 25° C. for additional 1 hour. The resulting mixture was purified directly by reversed-phase flash chromatography with the following conditions: column, C18 silica gel; mobile phase, MeCN in water (10 mmol/L NH₄HCO₃), 10% to 40% gradient in 20 min; detector, UV 254 nm to afford (2S)-N1-[4-methyl-5-(4-nitrophenyl)-1,3-thiazol-2-yl]pyrrolidine-1,2-dicarboxamide (500 mg, 52.21% yield) as a white solid.

LCMS (ESI): [M+H]⁺=376.10.

¹H NMR (400 MHz, DMSO-d₆) δ 11.07 (s, 1H), 8.27 (d, J=8.9 Hz, 2H), 7.70 (d, J=8.9 Hz, 2H), 7.39 (s, 1H), 6.96 (s, 1H), 4.30 (s, 1H), 3.62-3.60 (m, 1H), 3.50-3.44 (m, 1H), 2.43 (s, 3H), 2.13-2.08 (m, 1H), 1.91-1.82 (m, 3H).

### 3. Synthesis of Compound-50

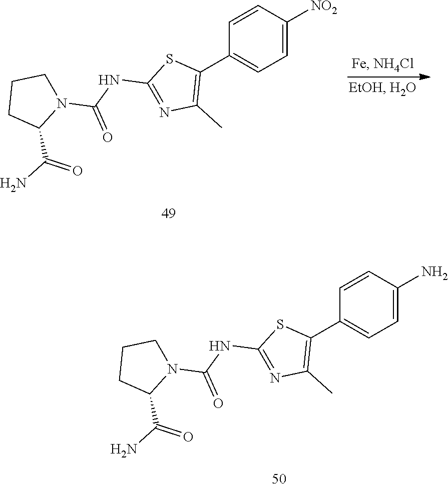 [SMILES: Cc1nc(NC(=O)N2CCC[C@H]2C(N)=O)sc1-c1ccc([N+](=O)[O-])cc1 ; Cc1nc(NC(=O)N2CCC[C@H]2C(N)=O)sc1-c1ccc(N)cc1]

### (2S)-N1-[5-(4-aminophenyl)-4-methyl-1,3-thiazol-2-yl]pyrrolidine-1,2-dicarboxamide

To a stirred solution of (2S)-N1-[4-methyl-5-(4-nitrophenyl)-1,3-thiazol-2-yl]pyrrolidine-1,2-dicarboxamide (250 mg, 0.67 mmol, 1.0 equiv.) and Fe (372 mg, 6.7 mmol, 10.0 equiv.) in EtOH (3 mL) and H2O (3 mL) was added ammonium chloride (356 mg, 6.7 mmol, 10.0 equiv.) in one portion at 25° C. The resulting mixture was heated to 80° C. and stirred at 80° C. for 30 minutes. The mixture was cooled to 25° C. After filtration, the filter cake was washed with MeOH (3×20 mL). The filtrate was concentrated under reduced pressure. The residue was purified by reversed-phase flash chromatography with the following conditions: column, C18 silica gel; mobile phase, MeCN in water (10 mmol/L NH4HCO3), 10% to 50% gradient in 10 min; detector, UV 254 nm, to afford (2S)-N1-[5-(4-aminophenyl)-4-methyl-1,3-thiazol-2-yl]pyrrolidine-1,2-dicarboxamide (150 mg, 65.21% yield) as a white solid.

LCMS (ESI): [M+H]⁺=346.15.

1H NMR (400 MHz, DMSO-d6) δ 7.34 (s, 1H), 7.05 (d, J=7.9 Hz, 2H), 6.93 (s, 1H), 6.60 (d, J=8.1 Hz, 2H), 6.03 (s, 1H), 5.22 (s, 2H), 4.26 (s, 1H), 3.58 (s, 1H), 3.43 (s, 1H), 2.23 (s, 3H), 2.09 (s, 1H), 1.86 (s, 3H).

4. Synthesis of b7a4b5

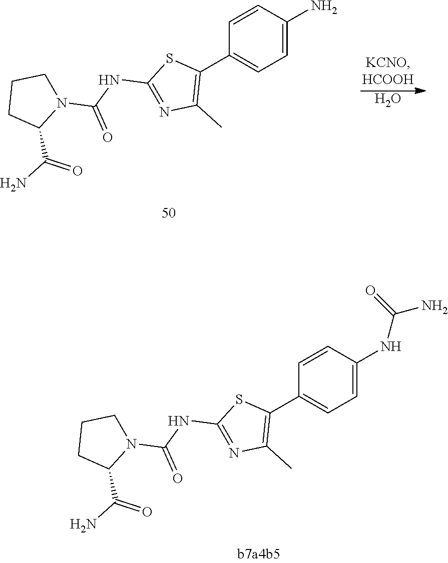 [SMILES: Cc1nc(NC(=O)N2CCC[C@H]2C(N)=O)sc1-c1ccc(N)cc1 ; O ; Cc1nc(NC(=O)N2CCC[C@H]2C(N)=O)sc1-c1ccc(NC(N)=O)cc1]

### (2S)-N1-(5-[4-(carbamoylamino)phenyl]-4-methyl-1,3-thiazol-2-ylpyrrolidine-1,2-dicarboxamide

To a stirred mixture of (2S)-N1-[5-(4-aminophenyl)-4-methyl-1,3-thiazol-2-yl]pyrrolidine-1,2-dicarboxamide (120 mg, 0.35 mmol, 1.0 equiv.) and acetic acid (63 mg, 1.0 mmol, 3. equiv.) in H₂O (4 mL) was added potassium cyanate (34 mg, 0.4 mmol, 1.2 equiv.) in one portion at 25° C. The resulting mixture was stirred at 25° C. for 4 hours. The resulting mixture was purified directly by reverse phase flash with the following conditions (Column: SunFire C18 5 μm, 30 mm×150 mm; Mobile Phase A: water (0.1% FA), Mobile Phase B: ACN; Flow rate: 60 mL/min; Gradient: 8% B to 25% B in 7 min; Wave Length: 254 nm/220 nm; RT1(min): 6.83) to afford (2S)-N1-(5-[4-(carbamoylamino)phenyl]-4-methyl-1,3-thiazol-2-ylpyrrolidine-1,2-dicarboxamide (17.2 mg, 12.81% yield) as a white solid.

LCMS (ESI): [M+H]⁺=389.00.

¹H NMR (400 MHz, DMSO-d₆) δ 8.66 (s, 1H), 8.17 (s, 1H), 7.45 (d, J=8.5 Hz, 2H), 7.35 (s, 1H), 7.27-7.25 (m, 2H), 6.94 (s, 1H), 5.89 (s, 2H), 4.27 (d, J=7.8 Hz, 1H), 3.61-3.56 (m, 1H), 3.47-3.41 (m, 1H), 2.28 (s, 3H), 2.10-2.05 (m, 1H), 1.89-1.84 (m, 3H).

¹³C NMR (101 MHz, DMSO) δ 174.57, 170.30, 163.92, 158.57, 156.40, 153.98, 140.01, 129.77, 129.10, 125.48, 122.92, 118.43, 60.31, 46.88, 30.43, 24.44, 15.95.

Procedure for the Synthesis of d0fd9a-0

### 1. Synthesis of Compound-52

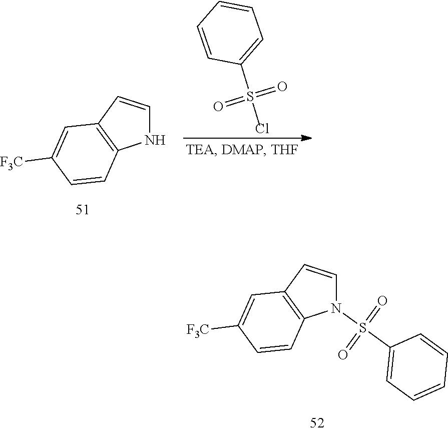

### 1-(benzenesulfonyl)-5-(trifluoromethyl)indole

A solution of 5-(trifluoromethyl)-1H-indole (4.6 g, 25.1 mmol, 1.0 equiv.) in THF (50 mL) was treated with benzenesulfonyl chloride (5.5 g, 31.4 mmol, 1.3 equiv.) at 25° C. followed by the addition of DMAP (0.3 g, 2.5 mmol, 0.1 equiv.) and TEA (7.6 g, 75.3 mmol, 3.0 equiv.) at 25° C. The reaction mixture was stirred at 25° C. for 2 hours under nitrogen atmosphere. The resulting mixture was purified directly by silica gel column chromatography, eluted with PE: EA (10:1) to afford 1-(benzenesulfonyl)-5-(trifluoromethyl)indole (3.4 g, 41.61% yield) as a light brown solid.

LCMS (ESI): [M−H]-=323.90.

¹H NMR (400 MHz, DMSO-d₆) δ 8.19-8.13 (m, 1H), 8.08-8.00 (m, 4H), 7.77-7.58 (m, 4H), 7.00 (dd, J=3.6, 0.8 Hz, 1H).

### 2. Synthesis of Compound-53

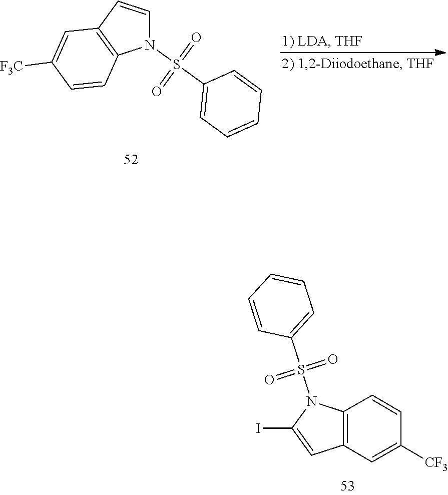 [SMILES: O=S(=O)(c1ccccc1)n1ccc2cc(C(F)(F)F)ccc21 ; O=S(=O)(c1ccccc1)n1c(I)cc2cc(C(F)(F)F)ccc21]

### 1-(benzenesulfonyl)-2-iodo-5-(trifluoromethyl)indole

To a stirred solution of 1-(benzenesulfonyl)-5-(trifluoromethyl)indole (3.0 g, 9.2 mmol, 1.0 equiv.) in THF (30 mL) was added LDA (2.0 μM in THF) (1 mL, 18.4 mmol, 2.0 equiv.) dropwise at −78° C. under nitrogen atmosphere. The reaction mixture was stirred at 25° C. for 2 hours under nitrogen atmosphere. To the above mixture was added 1,2-Diiodoethane (5.2 g, 18.4 mmol, 2.0 equiv.) dropwise at −78° C. and stirred at 25° C. for additional 1 hour. The reaction was warmed to 0° C. quenched by saturated NH₄Cl solution at 0° C. The resulting mixture was extracted with CH₂Cl₂ (5×20 mL). The combined organic layers were washed with water (5×10 mL), dried over anhydrous Na₂SO₄. After filtration, the filtrate was concentrated under reduced pressure. The residue was purified by silica gel column chromatography, eluted with PE/EA (10/1) to afford 1-(benzenesulfonyl)-2-iodo-5-(trifluoromethyl)indole (2.2 g, 52.91% yield) as a light yellow solid.

LCMS (ESI): [M−H]⁻=450.10.

¹H NMR (400 MHz, Chloroform-d) δ 8.40 (d, J=8.9 Hz, 1H), 7.93-7.90 (m, 2H), 7.71-7.70 (m, 1H), 7.62-7.58 (m, 1H), 7.54-7.46 (m, 3H), 7.06 (d, J=0.8 Hz, 1H).

### 3. Synthesis of Compound-54

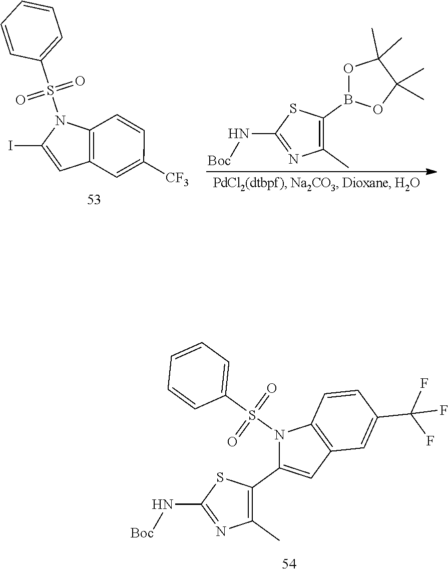 [SMILES: O=S(=O)(c1ccccc1)n1c(I)cc2cc(C(F)(F)F)ccc21 ; Cc1nc(NC(=O)OC(C)(C)C)sc1B1OC(C)(C)C(C)(C)O1 ; Cc1nc(NC(=O)OC(C)(C)C)sc1-c1cc2cc(C(F)(F)F)ccc2n1S(=O)(=O)c1ccccc1]

### tert-butyl N-{5-[1-(benzenesulfonyl)-5-(trifluoromethyl)indol-2-yl]-4-methyl-1,3-thiazol-2-yl}carbamate

To a stirred mixture of 1-(benzenesulfonyl)-2-iodo-5-(trifluoromethyl)indole (750 mg, 1.7 mmol, 1.0 equiv.) and tert-butyl N-[4-methyl-5-(4,4,5,5-tetramethyl-1,3,2-dioxaborolan-2-yl)-1,3-thiazol-2-yl]carbamate (1.1 g, 3.3 mmol, 2.0 equiv.) in dioxane (8 mL) and H₂O (2 mL) were added Pd(dtbpf)Cl₂ (108.3 mg, 0.2 mmol, 0.1 equiv.) and Na₂CO₃ (352 mg, 3.3 mmol, 2.0 equiv.) at 25° C. under nitrogen atmosphere. The reaction mixture was heated to 100° C. and stirred for 1.5 hours under nitrogen atmosphere. The mixture was cooled to 25° C. and purified directly by reversed-phase flash chromatography with the following conditions: column, C18 silica gel; mobile phase, MeCN in water (0.1% FA), 40% to 70% gradient in 30 minutes; detector, UV 254 nm to afford tert-butyl N-{5-[1-(benzenesulfonyl)-5-(trifluoromethyl)indol-2-yl]-4-methyl-1,3-thiazol-2-yl}carbamate (150 mg, 16.81% yield) as a yellow solid.

LCMS (ESI): [M+H]⁺=538.15.

¹H NMR (400 MHz, DMSO-d₆) δ 11.60 (s, 1H), 8.38 (d, J=8.8 Hz, 1H), 8.05-8.00 (m, 1H), 7.76 (dd, J=8.9, 2.0 Hz, 1H), 7.70-7.66 (m, 1H), 7.54 (d, J=4.3 Hz, 4H), 7.01 (d, J=0.7 Hz, 1H), 1.90 (s, 3H), 1.51 (s, 9H).

### 4. Synthesis of Compound-55

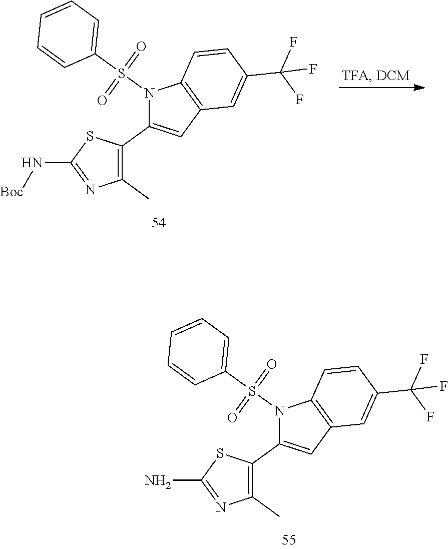 [SMILES: Cc1nc(NC(=O)OC(C)(C)C)sc1-c1cc2cc(C(F)(F)F)ccc2n1S(=O)(=O)c1ccccc1 ; Cc1nc(N)sc1-c1cc2cc(C(F)(F)F)ccc2n1S(=O)(=O)c1ccccc1]

### 5-[1-(benzenesulfonyl)-5-(trifluoromethyl)indol-2-yl]-4-methyl-1,3-thiazol-2-amine

A solution of tert-butyl N-{5-[1-(benzenesulfonyl)-5-(trifluoromethyl)indol-2-yl]-4-methyl-1,3-thiazol-2-yl}carbamate (140 mg, 0.3 mmol, 1.0 equiv.) in DCM (2 mL) and TFA (1 mL) was stirred at 25° C. for 1 hour. The resulting mixture was concentrated under reduced pressure. The residue was purified directly by reversed-phase flash chromatography with the following conditions: column, C18 silica gel; mobile phase, MeCN in water (10 mmol/L NH4HCO3), 10% to 70% gradient in 20 min; detector, UV 254 nm to afford 5-[1-(benzenesulfonyl)-5-(trifluoromethyl)indol-2-yl]-4-methyl-1,3-thiazol-2-amine (110 mg, 96.51% yield) as an off-white solid.

LCMS (ESI): [M+H]⁺=438.10.

1H NMR (400 MHz, DMSO-d6) δ 8.36 (d, J=8.8 Hz, 1H), 7.92 (d, J=1.9 Hz, 1H), 7.69-7.61 (m, 2H), 7.56-7.54 (m, 2H), 7.50-7.46 (m, 2H), 6.85 (s, 1H), 1.74 (s, 3H).

### 5. Synthesis of Compound-56

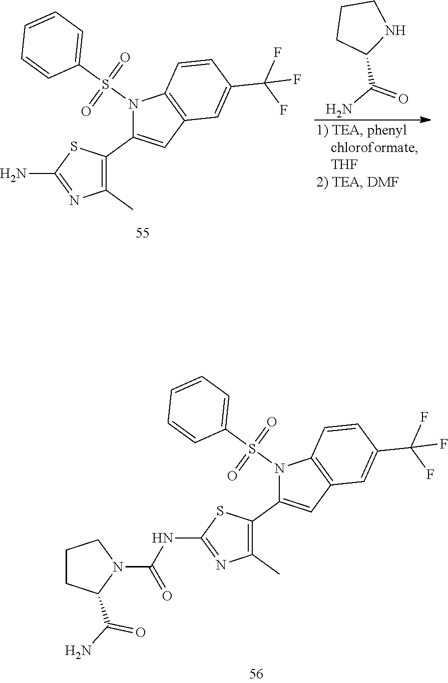 [SMILES: Cc1nc(N)sc1-c1cc2cc(C(F)(F)F)ccc2n1S(=O)(=O)c1ccccc1 ; NC(=O)[C@@H]1CCCN1 ; Cc1nc(NC(=O)N2CCC[C@H]2C(N)=O)sc1-c1cc2cc(C(F)(F)F)ccc2n1S(=O)(=O)c1ccccc1]

### (2S)-N1-{5-[1-(benzenesulfonyl)-5-(trifluoromethyl)indol-2-yl]-4-methyl-1,3-thiazol-2-yl}pyrrolidine-1,2-dicarboxamide

A solution of 5-[1-(benzenesulfonyl)-5-(trifluoromethyl)indol-2-yl]-4-methyl-1,3-thiazol-2-amine (100 mg, 0.2 mmol, 1.0 equiv.) in THF (1 mL) was treated with TEA (69 mg, 0.7 mmol, 3.0 equiv.) under nitrogen atmosphere followed by the addition of phenyl chloroformate (107 mg, 0.7 mmol, 3.0 equiv.) dropwise at 0° C. The reaction mixture was stirred at 25° C. for 20 minutes under nitrogen atmosphere. The resulting mixture was concentrated under reduced pressure. The residue was dissolved in DMF (1 mL). To above mixture was added TEA (69 mg, 0.7 mmol, 3.0 equiv.) and prolinamide (78 mg, 0.7 mmol, 3.0 equiv.) at 25° C. under nitrogen atmosphere. The reaction mixture was stirred at 25° C. for 2 hours under nitrogen atmosphere. The resulting mixture was purified directly by reversed-phase flash chromatography with the following conditions: column, C18 silica gel; mobile phase, MeCN in water (10 mmol/L NH4HCO3), 10% to 80% gradient in 30 minutes; detector, UV 254 nm to afford (2S)-N1-{5-[l-(benzenesulfonyl)-5-(trifluoromethyl)indol-2-yl]-4-methyl-1,3-thiazol-2-yl}pyrrolidine-1,2-dicarboxamide (90 mg, 68.21% yield) as an off-white solid.

LCMS (ESI): [M+H]⁺=578.05.

1H NMR (400 MHz, DMSO-d6) δ 10.43 (s, 1H), 8.38 (d, J=8.8 Hz, 1H), 8.01 (s, 1H), 7.75 (dd, J=9.0, 1.9 Hz, 1H), 7.68 (tt, J=5.4, 3.3 Hz, 1H), 7.54 (d, J=5.2 Hz, 2H), 7.40-7.36 (m, 2H), 6.98 (s, 2H), 4.30 (s, 1H), 3.62 (s, 1H), 3.48 (d, J=8.7 Hz, 1H), 2.12-2.07 (m, 1H), 1.91 (s, 3H), 1.88 (s, 2H), 1.23-1.12 (m, 2H).

6. Synthesis of d0fd9a-0

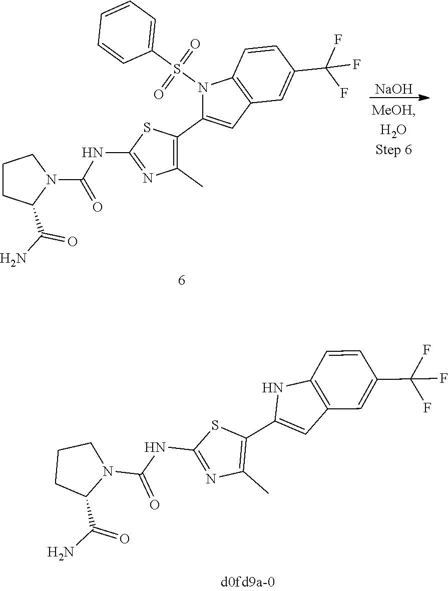

### (2S)-N1-{4-methyl-5-[5-(trifluoromethyl)-1H-indol-2-yl]-1,3-thiazol-2-yl}pyrrolidine-1,2-dicarboxamide

To a stirred solution of (2S)-N1-{5-[1-(benzenesulfonyl)-5-(trifluoromethyl)indol-2-yl]-4-methyl-1,3-thiazol-2-yl}pyrrolidine-1,2-dicarboxamide (60 mg, 0.1 mmol, 1.0 equiv.) in methanol (2 mL) were added 1N NaOH solution (0.6 mL) dropwise at 25° C. The reaction mixture was heated to 70° C. and stirred at 70° C. for 2 hours. The resulting mixture was cooled to 25° C. The resulting mixture was diluted with H₂O (10 mL) and extracted with EtOAc (3×10 mL). The combined organic layers were dried over anhydrous Na₂SO₄. After filtration, the filtrate was concentrated under reduced pressure. The crude product was purified by prep-HPLC with the following conditions (Column: YMC Triart C18 ExRs 5 μm, 30 mm*150 mm; Mobile Phase A: water (10 mmol/L NH₄HCO₃+0.1% NH₄OH), Mobile Phase B: ACN; Flow rate: 60 mL/min; Gradient: 34% B to 57% B in 8 min; Wave Length: 254/220 nm; RT1(min): 7.23) to afford (2S)-N1-{4-methyl-5-[5-(trifluoromethyl)-1H-indol-2-yl]-1,3-thiazol-2-yl}pyrrolidine-1,2-dicarboxamide (14.2 mg, 31.2% yield) as a white solid.

LCMS (ESI): [M+H]⁺=438.15.

¹H NMR (400 MHz, DMSO-d₆) δ 11.71 (s, 1H), 10.88 (s, 1H), 7.90 (s, 1H), 7.53 (d, J=8.5 Hz, 1H), 7.42-7.33 (m, 2H), 6.96 (s, 1H), 6.68 (s, 1H), 4.30 (s, 1H), 3.63-3.60 (m, 1H), 3.50-3.44 (m, 1H), 2.45 (s, 3H), 2.11-2.07 (m, 1H), 1.87 (t, J=6.4 Hz, 3H).

¹³C NMR (101 MHz, DMSO) δ 174.47, 138.63, 133.04, 130.08, 128.39, 127.38, 124.69, 121.18, 120.87, 120.57, 120.26, 118.01, 117.98, 117.56, 117.52, 112.25, 101.70, 60.39, 46.93, 30.42, 24.45, 16.75.

### Procedure for the Synthesis of Target 3-1

### 1. Synthesis of Target 3-1

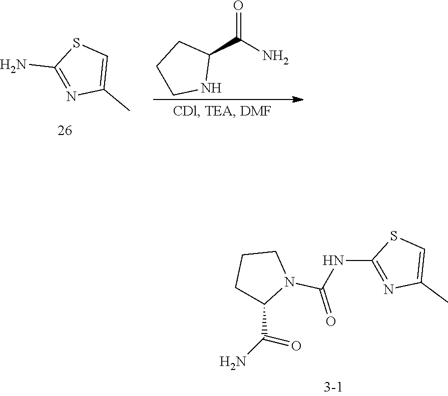

### (S)-N1-(4-methylthiazol-2-yl)pyrrolidine-1,2-dicarboxamide

A solution of 4-methylthiazol-2-amine (100 mg, 0.9 mmol, 1.0 equiv.) and 1-[(1H-imidazol-1-yl)carbonyl]-1H-imidazole (426 mg, 2.6 mmol, 3.0 equiv.) in DMF (1.5 mL) was stirred at 25° C. for 2 hours under nitrogen atmosphere. The resulting mixture was concentrated under vacuum. The mixture dissolve in DMF (2 mL) was treated with TEA (266 mg, 2.6 mmol, 3.0 equiv.) at 25° C. followed by the addition of 1-[(1H-imidazol-1-yl)carbonyl]-1H-imidazole (0.2 g, 1.3 mmol, 1.5 equiv.) in one portion at 25° C. The resulting mixture was heated to 70° C. and stirred at 70° C. for 2 hours. The resulting mixture was cooled down to 25° C. The resulting mixture was extracted with CHCl3 (3×50 mL). The combined organic layers were washed with brine (3×50 mL), dried over anhydrous Na2SO4. After filtration, the filtrate was concentrated under reduced pressure. The crude product was purified by prep-HPLC with the following conditions (Column: Mobile Phase A: Hex (0.1% DEA): (MeOH:DCM=1:1)=70:30; Flow rate: 1.0 mL/min; Gradient: isocratic; Injection Volume: 1 L) to afford (2S)-N1-(4-methyl-1,3-thiazol-2-yl)pyrrolidine-1,2-dicarboxamide (15.7 mg, 7.11% yield) as an off-white solid.

LCMS (ESI): [M+H]⁺=255.10.

1H NMR (400 MHz, DMSO-d6) δ 10.57 (s, 1H), 7.34 (s, 1H), 6.93 (s, 1H), 6.54 (s, 1H), 4.27-4.25 (m, 1H), 3.60-3.54 (m, 1H), 3.45-3.39 (m, 1H), 2.21 (d, J=1.1 Hz, 3H), 2.11-2.04 (m, 1H), 1.90-1.80 (m, 3H).

13C NMR (100 MHz, DMSO) δ 174.56, 160.72, 153.52, 146.39, 106.68, 60.25, 46.82, 30.42, 24.43, 17.19.

### Procedure for the Synthesis of Target 3-2

### 1. Synthesis of Compound-58

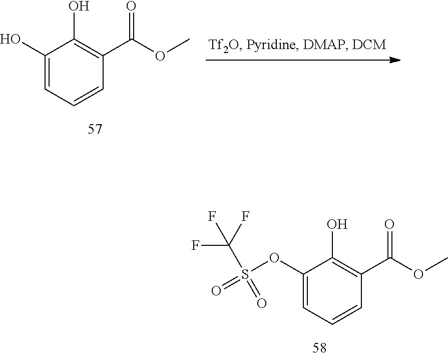 [SMILES: COC(=O)c1cccc(O)c1O ; COC(=O)c1cccc(OS(=O)(=O)C(F)(F)F)c1O]

### methyl 2-hydroxy-3-(((trifluoromethyl)sulfonyl)oxy)benzoate

To a stirred solution of methyl 2,3-dihydroxybenzoate (2.0 g, 11.9 mmol, 1.0 equiv.) and Pyridine (1.41 g, 17.8 mmol, 1.5 equiv.) in DCM (10 mL) were added Tf₂O (6.7 g, 23.7 mmol, 2.0 equiv.) and DMAP (0.7 g, 5.9 mmol, 0.5 equiv.) in two portions at 0° C. under nitrogen atmosphere. The resulting mixture was stirred at 0° C. for 2 hours under nitrogen atmosphere. The resulting mixture was washed with water (3×30 mL). The resulting mixture was extracted with EtOAc (3×30 mL). The combined organic layers were washed with brine (3×30 mL), dried over anhydrous Na₂SO₄. After filtration, the filtrate was concentrated under reduced pressure. This resulted in methyl 2-hydroxy-3-(trifluoromethanesulfonyloxy) benzoate (1.8 g, crude) as a yellow solid.

LCMS (ESI): [M−H]-=298.95.

¹H NMR (400 MHz, DMSO-d₆) δ 11.00 (s, 1H), 8.19-8.15 (m, 1H), 7.89-7.71 (m, 2H) 3.93 (d, J=1.6 Hz, 3H).

### 2. Synthesis of Compound-59

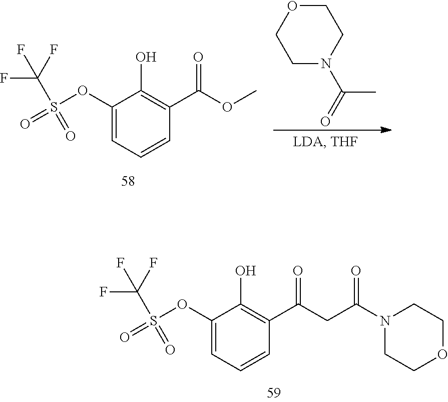

### 2-hydroxy-3-(3-morpholino-3-oxopropanoyl)phenyl trifluoromethanesulfonate

To a stirred solution of morpholine, 4-acetyl- (1.8 g, 13.9 mmol, 1.0 equiv.) in THF (50 mL) was added LDA (1.9 g, 18.0 mmol, 1.3 equiv.) dropwise at 0° C. under nitrogen atmosphere. The resulting mixture was stirred at 0° C. for 1 hour under nitrogen atmosphere. To the above mixture was added methyl 2-hydroxy-3-(trifluoromethanesulfonyloxy)benzoate (5.0 g, 16.7 mmol, 1.2 equiv.) in THE dropwise over 2 minutes at 0° C. The resulting mixture was heated to 25° C. and stirred at 25° C. for 2 hours under nitrogen atmosphere. The resulting mixture was extracted with EtOAc (3×30 mL). The combined organic layer was washed with brine (3×30 mL), dried over anhydrous Na₂SO₄. After filtration, the filtrate was concentrated under reduced pressure. This resulted in 2-hydroxy-3-[3-(morpholin-4-yl)-3-oxopropanoyl]phenyl trifluoromethanesulfonate (6.4 g, crude) as a yellow oil.

LCMS (ESI): [M+H]⁺=398.10.

¹H NMR (400 MHz, DMSO-d₆) δ 11.00 (s, 1H), 7.96-7.93 (m, 1H), 7.70-7.68 (m, 1H), 7.01-6.97 (m, 1H), 4.35 (s, 2H), 3.62-3.60 (m, 4H), 3.47-3.45 (m, 4H).

### 3. Synthesis of Compound-60

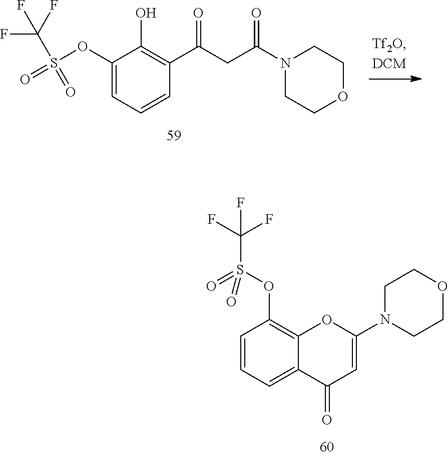 [SMILES: O=C(CC(=O)N1CCOCC1)c1cccc(OS(=O)(=O)C(F)(F)F)c1O ; O=c1cc(N2CCOCC2)oc2c(OS(=O)(=O)C(F)(F)F)cccc12]

### 2-morpholino-4-oxo-4H-chromen-8-yl trifluoromethanesulfonate

To a stirred solution of 2-hydroxy-3-[3-(morpholin-4-yl)-3-oxopropanoyl]phenyl trifluoromethanesulfonate (1.0 g, 2.5 mmol, 1.0 equiv.) in DCM (10 mL) was added Tf₂O (1.4 g, 5.0 mmol, 2.0 equiv.) dropwise at 0° C. under nitrogen atmosphere. The resulting mixture was heated to 25° C. and stirred at 25° C. for 2 hours under nitrogen atmosphere. The resulting mixture was extracted with EtOAc (3×30 mL). The combined organic layers were washed with brine (3×30 mL), dried over anhydrous Na₂SO₄. After filtration, the filtrate was concentrated under reduced pressure. The residue was purified by reversed-phase flash chromatography with the following conditions: column, C18 silica gel; mobile phase, MeCN in water (10 mmol/L NH₄HCO₃), 10% to 90% gradient in 10 min; detector, UV 254 nm. This resulted in 2-(morpholin-4-yl)-4-oxochromen-8-yl trifluoromethanesulfonate (820 mg, crude) as a yellow oil.

LCMS (ESI): [M+H]⁺=380.10.

¹H NMR (400 MHz, DMSO-d₆) δ 8.02-7.99 (m, 1H), 7.88-7.86 (m, 1H), 7.55-7.51 (m, 1H), 5.65 (s, 1H), 3.57-3.56 (m, 4H), 3.37-3.32 (m, 4H).

### 4. Synthesis of Target 3-2

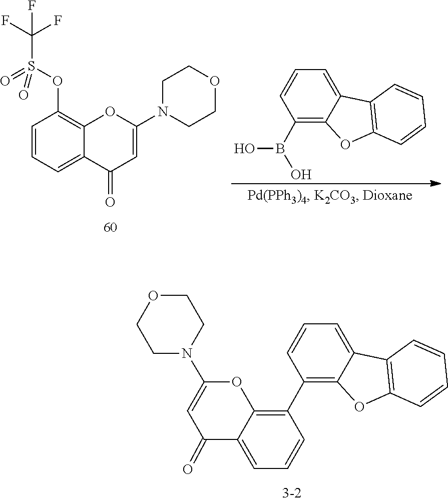 [SMILES: O=c1cc(N2CCOCC2)oc2c(OS(=O)(=O)C(F)(F)F)cccc12 ; OB(O)c1cccc2c1oc1ccccc12 ; O=c1cc(N2CCOCC2)oc2c(-c3cccc4c3oc3ccccc34)cccc12]

### 8-(dibenzo[b,d]furan-4-yl)-2-morpholino-4H-chromen-4-one

To a stirred solution of 2-(morpholin-4-yl)-4-oxochromen-8-yl trifluoromethanesulfonate (200 mg, 0.5 mmol, 1.0 equiv.) and dibenzo[b,d]furan-4-ylboronic acid (134 mg, 0.6 mmol, 1.2 equiv.) in dioxane (2.5 mL) were added K₂CO₃ (182 mg, 1.3 mmol, 2.5 equiv.) and Pd(PPh₃)₄ (61 mg, 0.1 mmol, 0.1 equiv.) in two portions at 0° C. under nitrogen atmosphere. The resulting mixture was heated to 90° C. and stirred at 90° C. for 2 hours under nitrogen atmosphere. The reaction mixture was cool down to 25° C. The resulting mixture was extracted with EtOAc (3×10 mL). The combined organic layers were washed with brine (3×10 mL), dried over anhydrous Na₂SO₄. After filtration, the filtrate was concentrated under reduced pressure. The crude product (150 mg) was purified by Prep-HPLC with the following conditions (Column: Agilent ZORBAX SB-Aq, 21.2*250 mm 7 m; Mobile Phase A: water (0.1% FA), Mobile Phase B: ACN; Flow rate: 25 mL/min; Gradient: 60% B to 65% B in 10 min; Wave Length: 254 nm/220 nm; RT1(min): 9.95) to afford 8-(dibenzo[b,d]furan-4-yl)-2-morpholino-4H-chromen-4-one (30.7 mg, 14.7% yield) as an off-white solid.

LCMS (ESI): [M+H]⁺=398.04.

¹H NMR (400 MHz, DMSO-d₆) δ 8.25 (dd, J=18.1, 7.7 Hz, 2H), 8.08 (d, J=7.7 Hz, 1H), 7.88 (d, J=7.4 Hz, 1H), 7.72 (d, J=8.2 Hz, 1H), 7.66 (d, J=7.4 Hz, 1H), 7.64-7.48 (m, 3H), 7.45 (t, J=7.5 Hz, 1H), 5.58 (s, 1H), 3.30 (t, J=4.9 Hz, 4H), 3.02 (t, J=4.9 Hz, 4H).

¹³C NMR (100 MHz, DMSO-d₆) δ 175.49, 162.21, 155.77, 153.19, 150.87, 134.39, 129.33, 128.36, 125.37, 125.33, 125.30, 124.38, 123.93, 123.86, 123.76, 123.47, 121.91, 121.85, 120.59, 112.26, 86.49, 65.40, 44.53.

### Dose-Response Analysis for PI3Kα of all Compounds

TABLE 4
Reagents for PI3Kα assays

| | Vendor |
|---|---|
| Materials and Reagents | |
| HEPES | Sigma |
| MgCl₂ | Sigma |
| EGTA | Sigma |
| CHAPS | Sigma |
| NaCl | Sigma |
| DTT | Merck |
| ATP(10 mM) | Promega |
| PI3Kα | Invitrogen |
| ADP-Glo Kinase Assay | Promega |
| PIP2:3PS | Promega |
| Consumable and Instrument | |
| ProxiPlate-384 Plus, White-shallow well | PerkinElmer |
| V96 Micro Well Plates | Nunc |
| Plate shaker | QILINBEIER |
| Centrifuge | Eppendorf |
| Multiplate reader | PerkinElmer |
| Echo | PE |
| Topseal A | PerkinElmer |

### Protocol

Candidate PI3Kα inhibitory compounds were characterized as follows:

### 1. Assay Procedure

1.1 Working stock preparation.

1) The reference compound BYL719 was diluted to 100 μM in DMSO. And then 3-fold serial diluted from 100 μM for 10 doses in DMSO.

2) The test compounds were diluted to 100 μM/2500 μM/100000 μM in DMSO. And then 3-fold serial diluted from 100 μM/2500 μM/100000 μM for 10 doses in DMSO.

3) 1% DMSO as Vehicle Control, 100 μM BYL719 as Positive Control.

1.2 Preparation reagent:

Prepare 1×Assay buffer solution, 2× Lipid buffer solution, 2×PI3K Working Solution, and 2× Substrate Working Solution.

1.3 Compound screening:

1) Add 40nl/well of compounds, vehicle control and positive control which prepared in step 1.1 to the 384-well using Echo.

2) Add 2 μl/well of 2×PI3Kα Working solution which prepared in step 1.2 to 384-well white ProxiPlate using multichannel pipettes.

3) Mix the compounds and PI3Kα working solution using a shaker for 30s, and incubate it at 25° C. for 15 min.

4) Add 2 μl/well of 2× Substrate working solution which prepared in step 1.2 to the 384-well plate to initiate reaction.

5) Seal the assay plate.

6) Incubate at 25° C. for 60 min.

7) Add 4 μl/well of ADP-Glo reagents buffer with 10 mM MgCl₂ (the stock concentration of MgCl₂ is 1 mol/L, 100-fold dilute it using ADP-Glo reagent) to the 384-well Plate using multichannel pipettes. Seal the assay plate and incubate at 25° C. for 40 min.

8) Add 8 μl/well of kinase detection reagents to the 384-well plate using multichannel pipettes. Seal the assay plate and incubate at 25° C. for 40 min.

9) Read on Envision 2105 for the value of RLU (relative luminescence unit).

### 2. Data Analysis

1) Luminescence signal (RLU) is detected for each well.

2) % Inhibition is calculated as follows:

$$\% Inhibition = ⌊ ⌊ 1 - \frac{{RLU}_{cmpd} - {\overline{RLU}}_{Positive}}{{\overline{RLU}}_{Vehicle} - {\overline{RLU}}_{Positive}} ⌋ ⌋ * 100$$

R̅L̅U̅ₚₒₛᵢₜᵢᵥₑ: The average RLU for the positive controls across the plate.
R̅L̅U̅_{vehicle}: The average RLU for negative controls across the plate.
   3) Calculate IC₅₀ and Plot Effect-Dose Curve of cmpds:

Calculate IC₅₀ by fitting % Inhibition values and log of compound concentrations to nonlinear regression (dose response−variable slope) with Graphpad Prism 8.

$$Y = Bottom + ( Top - Bottom ) / ( 1 + 1 {0}^{⋀} ( ( {LogIC}_{50} - X ) * HillSlope ) )$$

X: log of Inhibitor concentration; Y: % Inhibition.

### Assay:

Luminescence values were obtained with the ADP-Glo Assay (Promega) according to the instructions.

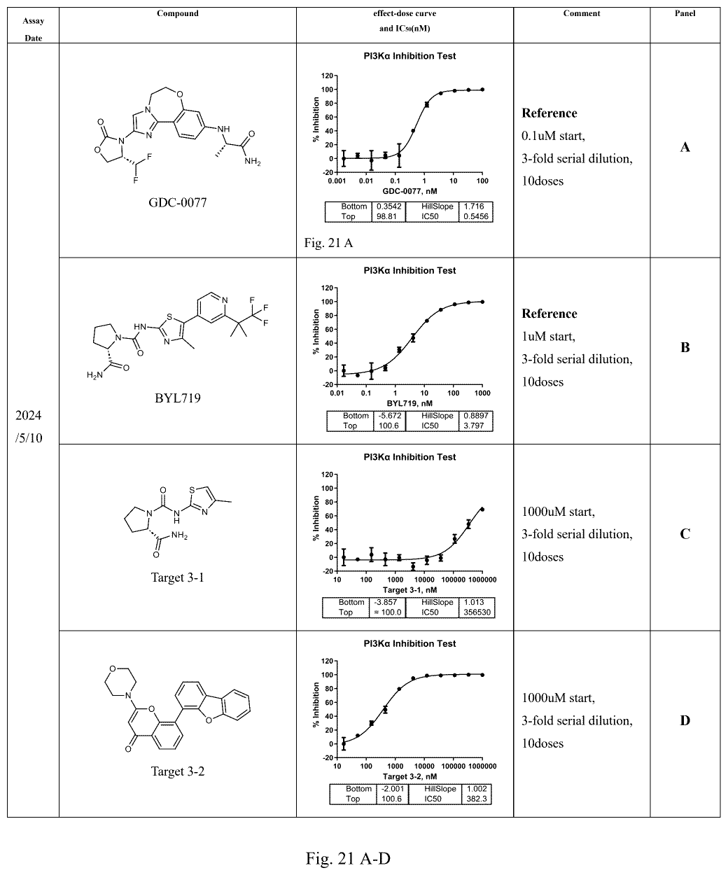

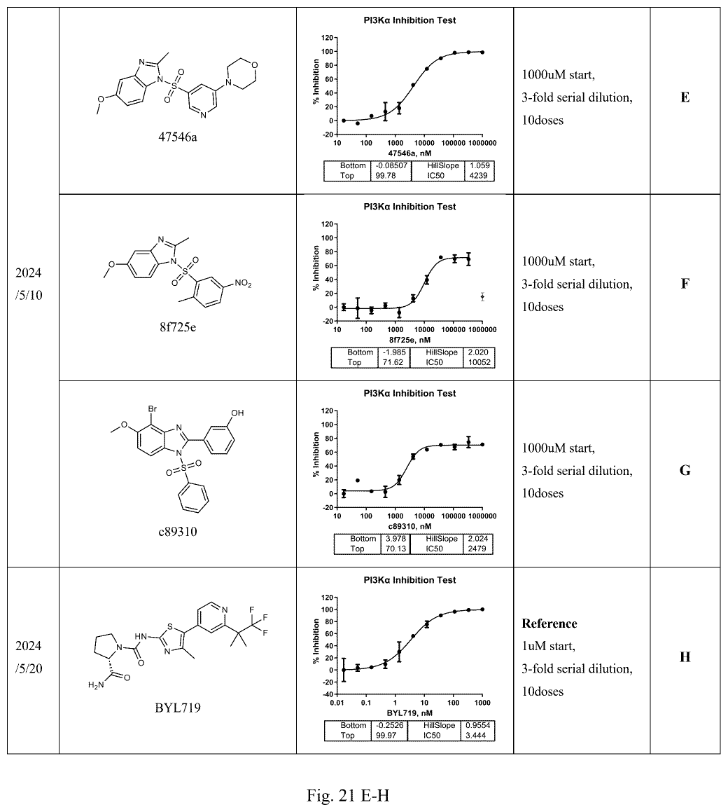

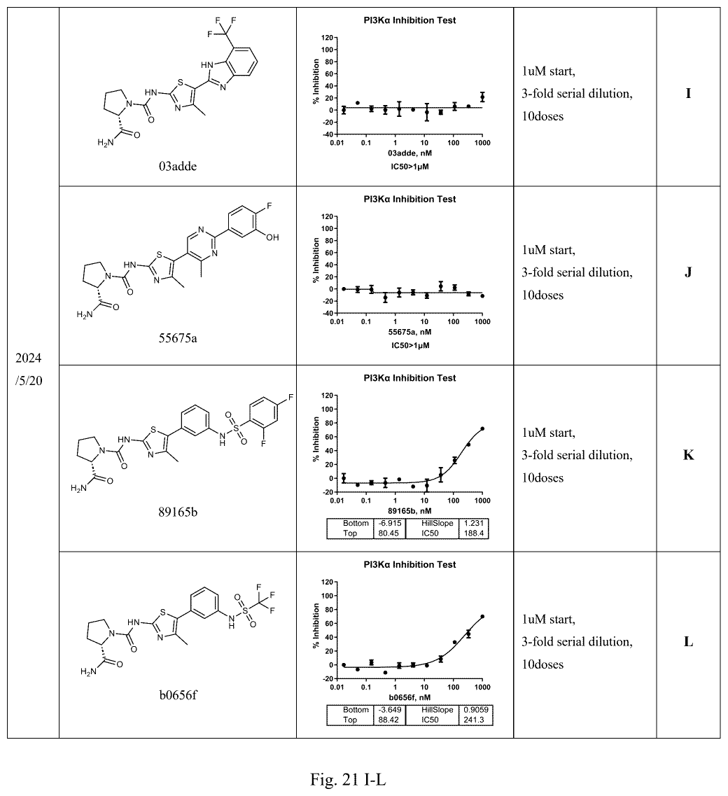

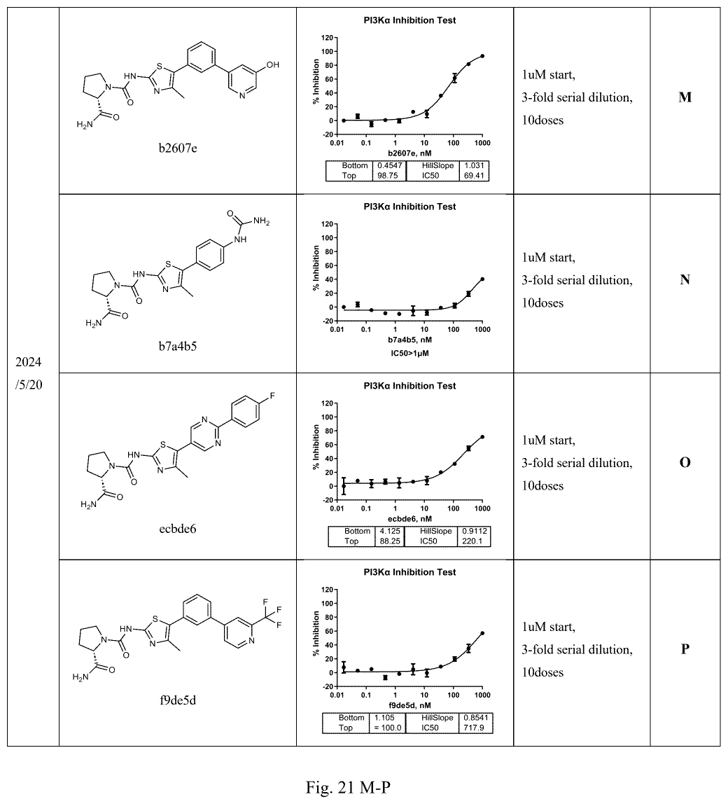

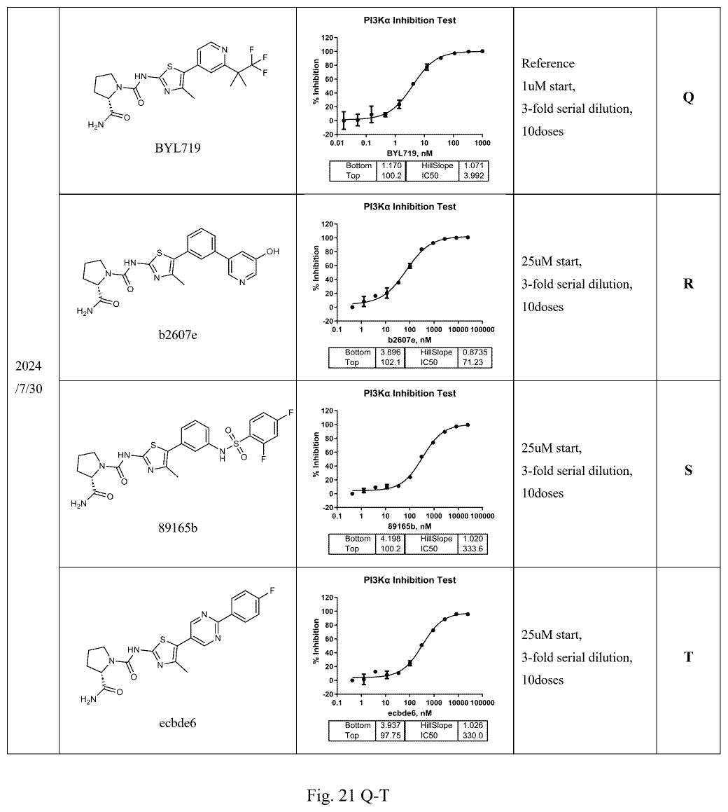

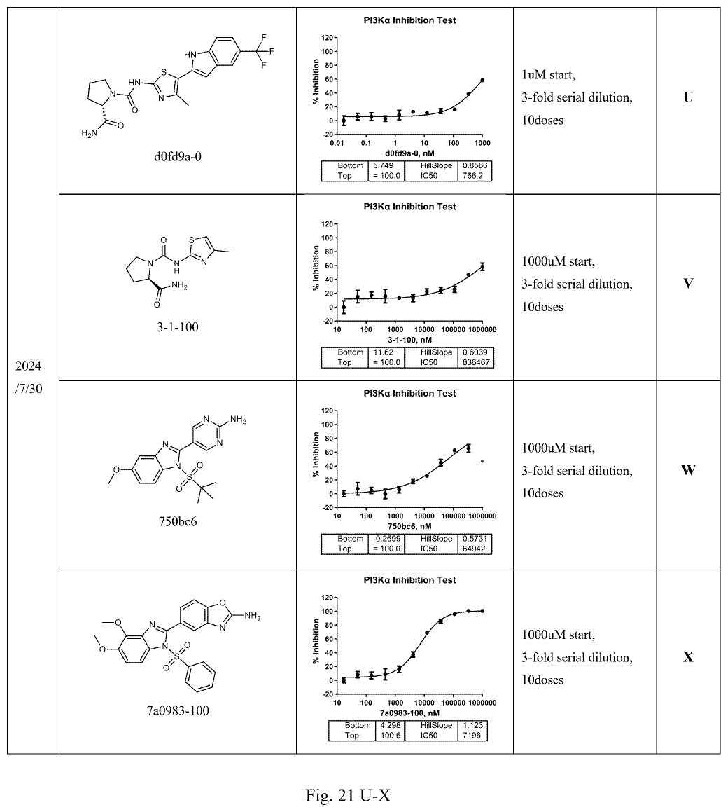

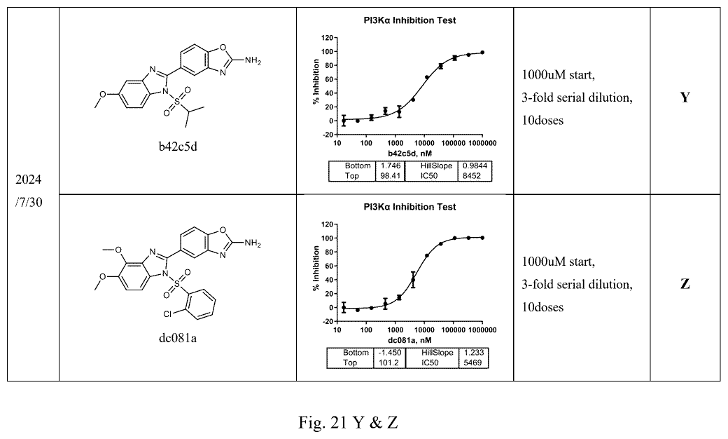

FIG. 21 shows IC₅₀ of AI-generated PI3Kα inhibitors. Standards and candidate compound were tested on three different days. Reference compound IC₅₀ values were ˜0.5 nM for GDC-0077 and ˜4 nM for BYL719, in line with their reported values. Candidate compound IC₅₀ values were as low as ˜70 nM for compounds b2607e and b2607e, with a number of other compounds in the 100-400 nM range (98165b, b0656f, ecbde6, and 89165b).

### Preferred Embodiments

In an embodiment, the compound of the claimed invention is a compound having the structure of Formula (I)

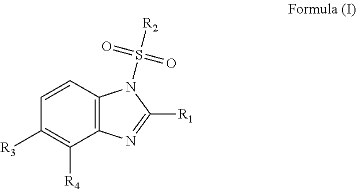

wherein R1 R1 is alkyl, alkenyl, alkynyl, alkoxy, amido, alkoxycarbonyl, sulfonamido, halo, cyano, or nitro, aryl, heteroaryl, heterocycloalkyl, or cycloalkyl; R2 is alkyl, alkenyl, alkynyl, alkoxy, amido, alkoxycarbonyl, sulfonamido, halo, cyano, or nitro, aryl, heteroaryl, heterocycloalkyl, or cycloalkyl, R 3 is halo, hydroxyl or alkoxy, and R4 is halo, hydroxyl or alkoxy.

R1 is alkyl, aryl, heteroaryl or heterocycloalkyl, wherein R2 is alkyl, aryl, heteroaryl or heterocycloalkyl, R 3 is halo, hydroxyl or alkoxy, and R4 is halo, hydroxyl or alkoxy.

In an embodiment, R1 is selected from:

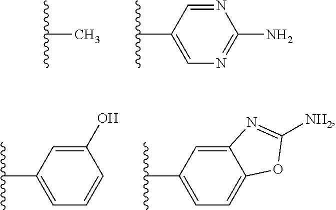

wherein R2 is selected from:

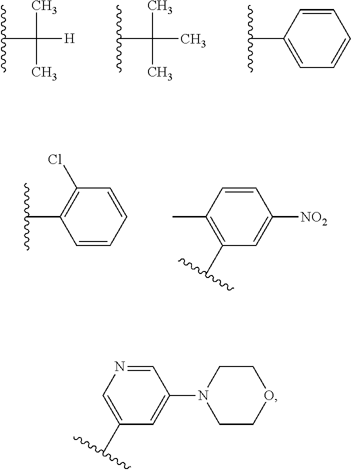

wherein R3 is selected from:

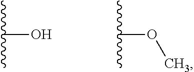

and wherein R4 is selected from:

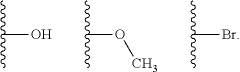

In an embodiment the compound is selected from:

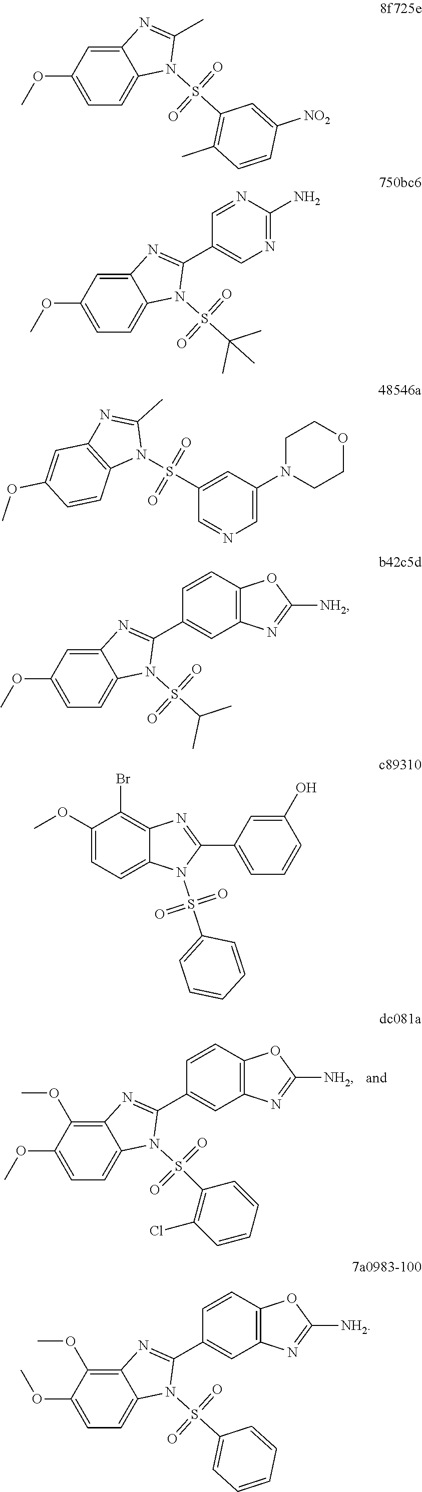

In an embodiment, the compound comprises a scaffold moiety comprising Target 3-1, having the structure:

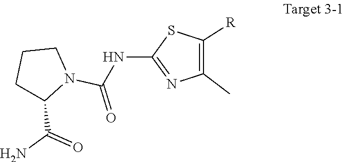

or a pharmaceutically acceptable salt thereof, or an isomer thereof, wherein: R is alkyl, alkenyl, alkynyl, alkoxy, amido, alkoxycarbonyl, sulfone, sulfonyl, sulfinyl, sulfonamido, halo, cyano, nitro, aryl, heteroaryl, heterocycloalkyl, or cycloalkyl.

In an embodiment R is alkyl, aryl, sulfonyl or heteroaryl.

In an embodiment, the compound is selected from:

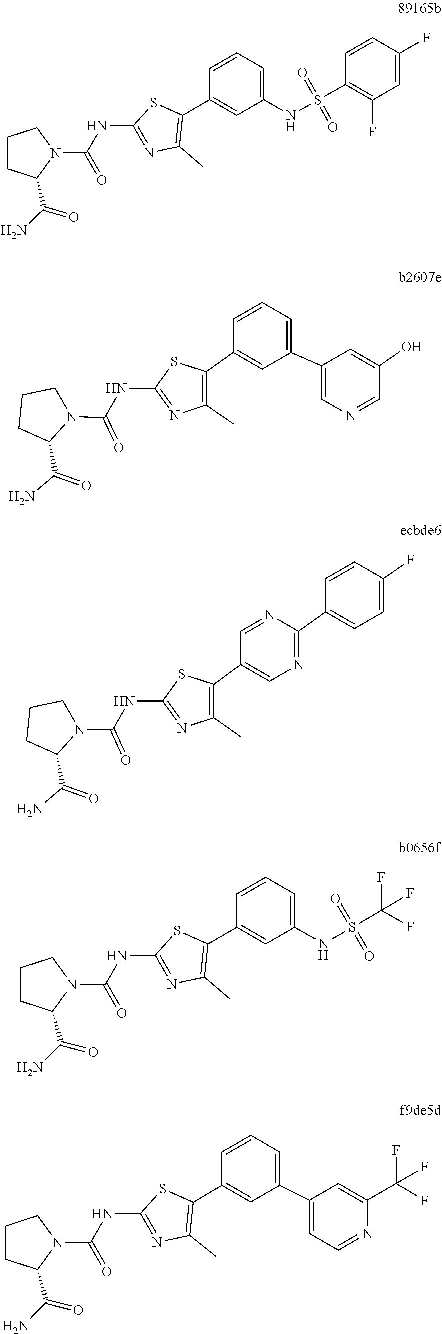 [SMILES: Cc1nc(NC(=O)N2CCC[C@H]2C(N)=O)sc1-c1cccc(NS(=O)(=O)c2ccc(F)cc2F)c1 ; Cc1nc(NC(=O)N2CCC[C@H]2C(N)=O)sc1-c1cccc(-c2cncc(O)c2)c1 ; Cc1nc(NC(=O)N2CCC[C@H]2C(N)=O)sc1-c1cnc(-c2ccc(F)cc2)nc1 ; Cc1nc(NC(=O)N2CCC[C@H]2C(N)=O)sc1-c1cccc(NS(=O)(=O)C(F)(F)F)c1 ; Cc1nc(NC(=O)N2CCC[C@H]2C(N)=O)sc1-c1cccc(-c2ccnc(C(F)(F)F)c2)c1]

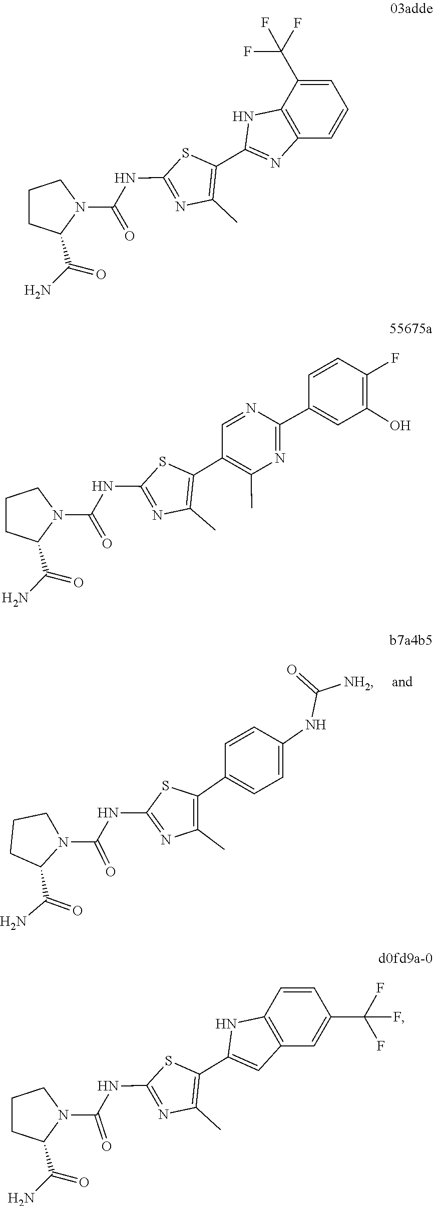 [SMILES: Cc1nc(NC(=O)N2CCC[C@H]2C(N)=O)sc1-c1nc2cccc(C(F)(F)F)c2[nH]1 ; Cc1nc(-c2ccc(F)c(O)c2)ncc1-c1sc(NC(=O)N2CCC[C@H]2C(N)=O)nc1C ; Cc1nc(NC(=O)N2CCC[C@H]2C(N)=O)sc1-c1ccc(NC(N)=O)cc1 ; Cc1nc(NC(=O)N2CCC[C@H]2C(N)=O)sc1-c1cc2cc(C(F)(F)F)ccc2[nH]1]

or a pharmaceutically acceptable salt thereof.

The present invention provides a novel class of PI3Kα inhibitors that exhibit exceptional therapeutic potential for the treatment, prevention, and management of a wide range of diseases and conditions associated with aberrant PI3Kα activity. These compounds are particularly promising for use in oncology, metabolic disorders, inflammatory diseases, and other conditions where PI3Kα signaling plays a critical role. The invention encompasses not only the compounds themselves but also their use in various therapeutic, diagnostic, and research applications, as well as their formulation into a diverse array of pharmaceutical compositions tailored for optimal delivery and efficacy.

### Therapeutic Applications

The PI3Kα inhibitors of the invention are designed to address unmet medical needs in the treatment of diseases such as:

Cancer: Including but not limited to breast cancer, ovarian cancer, prostate cancer, glioblastoma, colorectal cancer, pancreatic cancer, lung cancer, and hematologic malignancies such as leukemia and lymphoma.

Metabolic Disorders: Such as type 2 diabetes, obesity, and insulin resistance, where PI3Kα signaling is implicated in metabolic regulation.

Inflammatory and Autoimmune Diseases: Including rheumatoid arthritis, systemic lupus erythematosus, inflammatory bowel disease, and psoriasis.

Cardiovascular Diseases: Such as atherosclerosis, myocardial infarction, and heart failure, where PI3Kα modulation may offer therapeutic benefits.

Neurological Disorders: Including Alzheimer's disease, Parkinson's disease, and other neurodegenerative conditions.

### Combination Therapies

The PI3Kα inhibitors of the invention may be employed as standalone therapies or in combination with other therapeutic agents to enhance efficacy, reduce resistance, or minimize adverse effects. Potential combination therapies include, but are not limited to:

Chemotherapeutic Agents: Such as platinum-based compounds (e.g., cisplatin, carboplatin), taxanes (e.g., paclitaxel, docetaxel), anthracyclines (e.g., doxorubicin), and antimetabolites (e.g., 5-fluorouracil, gemcitabine).

Targeted Therapies: Including HER2 inhibitors (e.g., trastuzumab), EGFR inhibitors (e.g., erlotinib), PARP inhibitors (e.g., olaparib), CDK4/6 inhibitors (e.g., palbociclib), and BRAF inhibitors (e.g., vemurafenib).

Immunotherapies: Such as immune checkpoint inhibitors (e.g., anti-PD-1, anti-PD-L1, anti-CTLA-4 antibodies), CAR-T cell therapies, and cancer vaccines.

Hormonal Therapies: Including aromatase inhibitors (e.g., letrozole), selective estrogen receptor modulators (e.g., tamoxifen), and anti-androgens (e.g., enzalutamide).

Radiotherapy: Where the PI3Kα inhibitors may act as radiosensitizers to enhance the efficacy of radiation treatment.

### Diagnostic and Prognostic Uses

The compounds of the invention may also be utilized in diagnostic and prognostic applications, such as:

Biomarker Identification: To stratify patients based on PI3Kα expression or activity levels, enabling personalized treatment approaches.

Companion Diagnostics: To determine the suitability of patients for PI3Kα inhibitor therapy and monitor treatment response.

Imaging Agents: Conjugated PI3Kα inhibitors may be used in imaging techniques (e.g., PET, MRI) to visualize tumors or other disease sites.

Monitoring Disease Progression: To assess the effectiveness of treatment and guide therapeutic adjustments.

### Pharmaceutical Formulations and Delivery Methods

The PI3Kα inhibitors of the invention may be formulated into a wide variety of pharmaceutical compositions to facilitate their administration via multiple routes. These formulations may be tailored to optimize bioavailability, stability, and patient compliance.

Examples include:

### Oral Formulations:

Tablets, capsules, and caplets designed for immediate or modified release.

Liquid formulations such as suspensions, syrups, or solutions.

Orally disintegrating tablets (ODTs) or films for patients with difficulty swallowing.

Enteric-coated formulations to protect the active ingredient from stomach acid.

### Parenteral Formulations:

Injectable solutions or suspensions for intravenous (IV), intramuscular (IM), or subcutaneous (SC) administration.

Lyophilized powders for reconstitution prior to injection.

Implantable devices or depots for sustained release over extended periods.

### Topical Formulations:

Creams, ointments, gels, or lotions for localized delivery to the skin or mucous membranes.

Transdermal patches for controlled, continuous delivery through the skin.

### Inhalable Formulations:

Aerosols, nebulizers, or dry powder inhalers for pulmonary delivery, particularly useful for lung cancers or systemic delivery.

### Controlled-Release and Sustained-Release Formulations:

Microspheres, nanoparticles, or liposomes for targeted delivery and prolonged release.

Hydrogels or biodegradable polymers for localized and sustained drug release.

### Nanotechnology-Based Formulations:

Nanocarriers such as lipid nanoparticles, polymeric nanoparticles, or dendrimers to enhance solubility, stability, and targeted delivery.

Conjugation with targeting ligands (e.g., antibodies, peptides) for site-specific delivery to tumors or diseased tissues.

### Combination Formulations:

Fixed-dose combinations incorporating PI3Kα inhibitors with other therapeutic agents in a single dosage form.

Co-formulations with excipients or adjuvants to enhance stability, absorption, or therapeutic efficacy.

### Additional Uses

Beyond therapeutic applications, the PI3Kα inhibitors of the invention may be employed in:

Research Tools: For studying PI3Kα signaling pathways, cellular processes, and disease mechanisms.

Drug Discovery: As reference compounds or leads for the development of next-generation PI3Kα inhibitors.

Personalized Medicine: Enabling tailored treatment strategies based on individual patient characteristics and biomarker profiles.

Specific art-recognized modalities of formulation and application of this class of compounds are set forth in U.S. Pat. No. 8,193,182 B2, U.S. Pat. No. 9,221,795 B2, U.S. Pat. No. 9,255,103 B2, 9,629,843, and 10,112,932, each of which is incorporated herein in its entirety.

In summary, the present invention provides a versatile and potent class of PI3Kα inhibitors with broad applicability in medicine, diagnostics, and research. The compounds, formulations, and methods described herein represent a significant advancement in the field, offering new opportunities for the treatment and management of diseases associated with PI3Kα dysregulation. The invention is further distinguished by its adaptability to a wide range of delivery methods and formulations, ensuring optimal therapeutic outcomes for diverse patient populations.

We claim:

1. A compound of Formula (I) 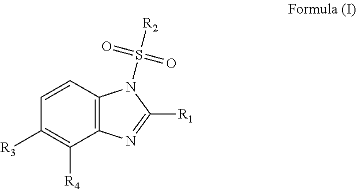
a pharmaceutically acceptable salt thereof, or an isomer thereof, wherein: R1 is alkyl, alkenyl, alkynyl, alkoxy, amido, alkoxycarbonyl, sulfonamido, halo, cyano, or nitro, aryl, heteroaryl, heterocycloalkyl, or cycloalkyl; R2 is alkyl, alkenyl, alkynyl, alkoxy, amido, alkoxycarbonyl, sulfonamido, halo, cyano, or nitro, aryl, heteroaryl, heterocycloalkyl, or cycloalkyl, R 3 is halo, hydroxyl or alkoxy, and R4 is halo, hydroxyl or alkoxy.

2. The compound of claim 1, wherein R1 is alkyl, aryl, heteroaryl or heterocycloalkyl, wherein R2 is alkyl, aryl, heteroaryl or heterocycloalkyl, R 3 is halo, hydroxyl or alkoxy, and R4 is halo, hydroxyl or alkoxy.

3. The compound of claim 2, wherein R1 is selected from: 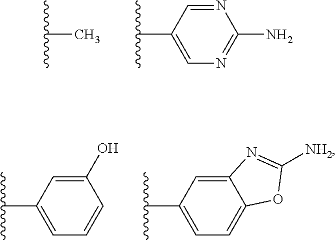
wherein R2 is selected from: 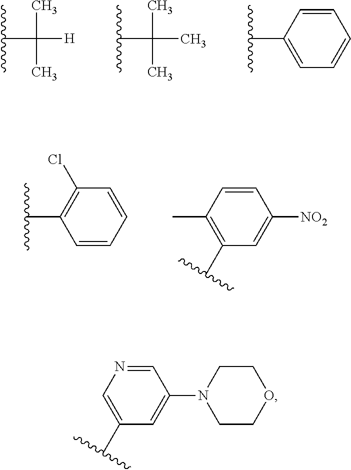
wherein R3 is selected from: 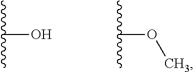
and wherein R4 is selected from: 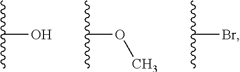
or a pharmaceutically acceptable salt thereof.

4. The compound of claim 3, wherein the compound is selected from: 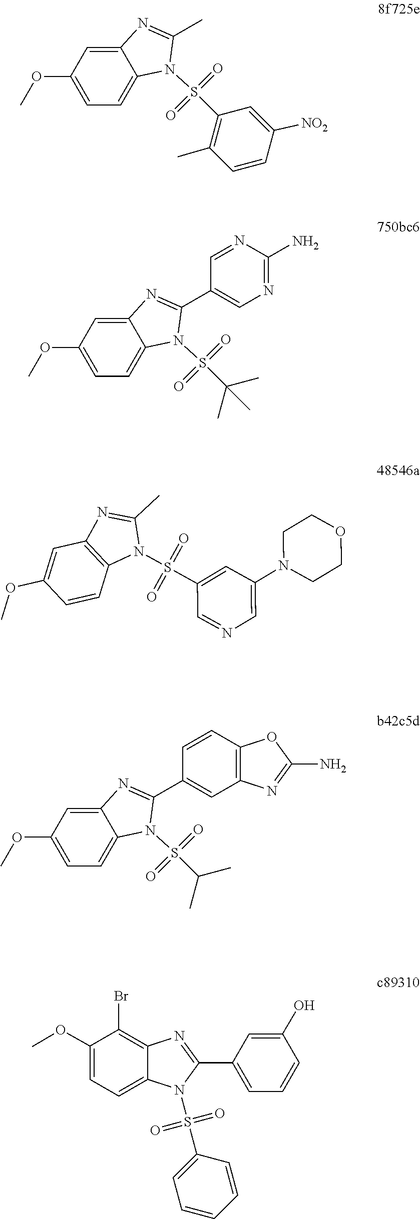 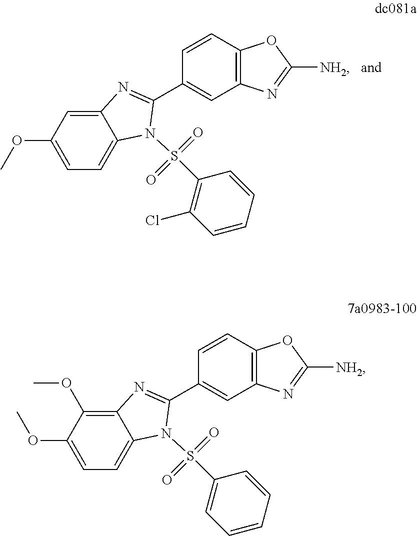 [SMILES: COc1ccc2c(c1)nc(-c1ccc3oc(N)nc3c1)n2S(=O)(=O)c1ccccc1Cl ; COc1ccc2c(nc(-c3ccc4oc(N)nc4c3)n2S(=O)(=O)c2ccccc2)c1OC]
or a pharmaceutically acceptable salt thereof.

5. The compound of claim 4, wherein the compound is 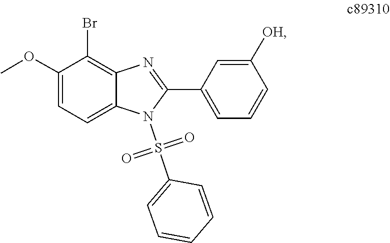 [SMILES: COc1ccc2c(nc(-c3cccc(O)c3)n2S(=O)(=O)c2ccccc2)c1Br]
or a pharmaceutically acceptable salt thereof.

6. A compound comprising Target 3-1 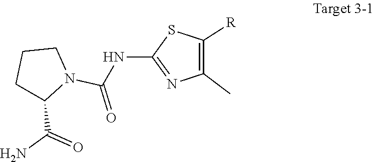
a pharmaceutically acceptable salt thereof, or an isomer thereof, wherein: R is alkyl, alkenyl, alkynyl, alkoxy, amido, alkoxycarbonyl, sulfone, sulfonyl, sulfinyl, sulfonamido, halo, cyano, nitro, aryl, heteroaryl, heterocycloalkyl, or cycloalkyl.

7. The compound of claim 6, wherein R is alkyl, aryl, sulfonyl or heteroaryl.

8. The compound of claim 7, wherein the compound is selected from: 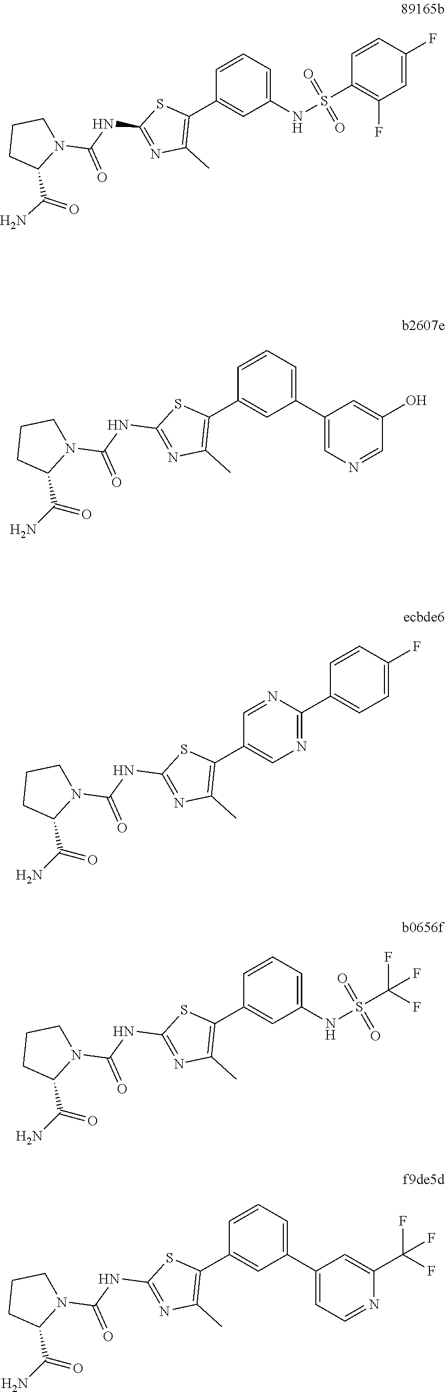 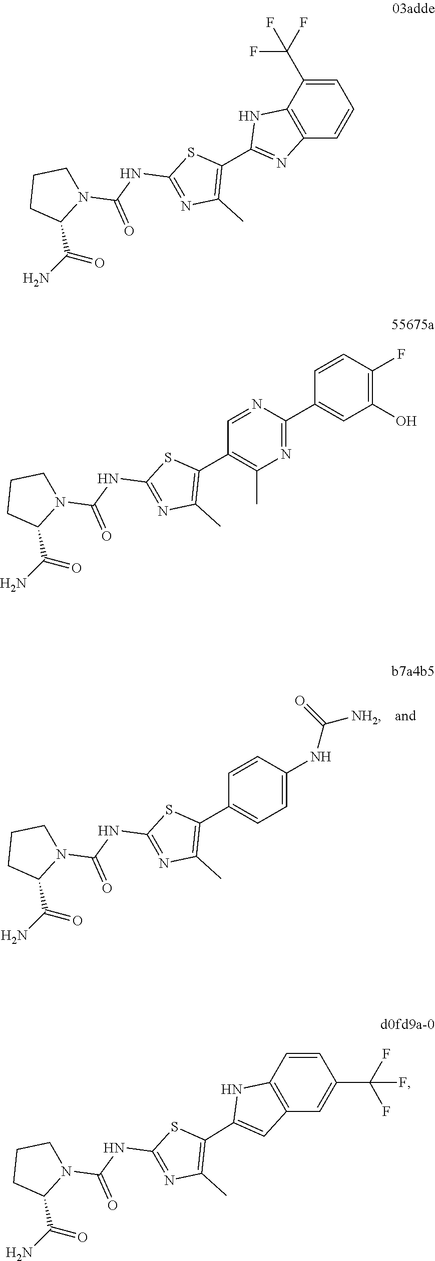 [SMILES: Cc1nc(NC(=O)N2CCC[C@H]2C(N)=O)sc1-c1nc2cccc(C(F)(F)F)c2[nH]1 ; Cc1nc(-c2ccc(F)c(O)c2)ncc1-c1sc(NC(=O)N2CCC[C@H]2C(N)=O)nc1C ; Cc1nc(NC(=O)N2CCC[C@H]2C(N)=O)sc1-c1ccc(NC(N)=O)cc1 ; Cc1nc(NC(=O)N2CCC[C@H]2C(N)=O)sc1-c1cc2cc(C(F)(F)F)ccc2[nH]1]
or a pharmaceutically acceptable salt thereof.

9. The compound of claim 8, where the compound is 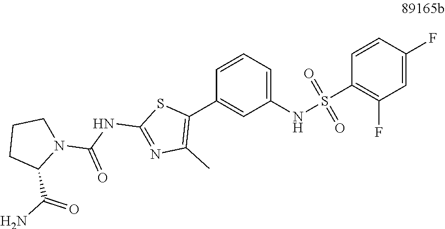 [SMILES: Cc1nc(NC(=O)N2CCC[C@H]2C(N)=O)sc1-c1cccc(NS(=O)(=O)c2ccc(F)cc2F)c1]
or a pharmaceutically acceptable salt thereof.
